(19) United States
(12) Patent Application Publication
(10) Pub. No.: US 2024/0218395 A1
(43) Pub. Date: Jul. 4, 2024

(54) ALPHAVIRUS VECTORS CONTAINING UNIVERSAL CLONING ADAPTORS

(71) Applicant: REPLICATE BIOSCIENCE, INC., San Diego, CA (US)

(72) Inventors: Nathaniel Stephen WANG, San Diego, CA (US); Shigeki Joseph MIYAKE-STONER, San Diego, CA (US); Annie Chia-Zong CHOU, San Diego, CA (US)

(21) Appl. No.: 18/555,852

(22) PCT Filed: Apr. 20, 2022
(86) PCT No.: PCT/US2022/025470

§ 371 Date: Oct. 17, 2023

Related U.S. Application Data
(60) Provisional application No. 63/177,656, filed on Apr. 21, 2021.

(51) Int. Cl. C12N 15/86; A61K 39/00; A61K 39/145; A61P 31/16; C07K 14/005

(52) U.S. Cl. CPC C12N 15/86; A61K 39/145; A61P 31/16; C07K 14/005; A61K 2039/5256; A61K 2039/53; A61K 2039/55555; C12N 2760/16034; C12N 2760/16071; C12N 2770/36143; C12N 2800/70; C12N 2830/42; C12N 2830/50; C12N 2840/203

(57) ABSTRACT

The present disclosure relates to the field of molecular virology, including nucleic acid molecules comprising modified viral genomes or replicons (e.g., self-replicating RNAs), pharmaceutical compositions containing the same, and the use of such nucleic acid molecules and compositions for production of desired products in cell cultures or in a living body. Also provided are methods for modulating an immune response in a subject in need thereof, as well as methods for preventing and/or treating various health conditions.

### CROSS-REFERENCE TO RELATED APPLICATIONS

This application claims the benefit of priority to U.S. Provisional Patent Application Ser. No. 63/177,656, filed on Apr. 21, 2021. The disclosure of the above-referenced application is herein expressly incorporated by reference it its entirety, including any drawings.

### FIELD

The present disclosure relates to the field of molecular virology and immunology, and particularly relates to nucleic acid molecules encoding modified viral genomes and replicons (e.g., self-replicating RNAs), pharmaceutical compositions containing the same, and the use of such nucleic acid molecules and compositions for production of desired products in cell cultures or in a living body. Also provided are methods for modulating an immune response in a subject in need thereof, as well as methods for preventing and/or treating various health conditions.

### INCORPORATION OF THE SEQUENCE LISTING

The material in the accompanying Sequence Listing is hereby incorporated by reference into this application. The accompanying Sequence Listing text file, named 058462-503001WO_Sequence_Listing.txt, was created on Apr. 12, 2022, and is 227 KB.

### BACKGROUND

In recent years, several different groups of animal viruses have been subjected to genetic manipulation either by homologous recombination or by direct engineering of their genomes. The availability of reverse genetics systems for both DNA and RNA viruses has created new perspectives for the use of recombinant viruses, for example, as vaccines, expression vectors, anti-tumor agents, gene therapy vectors, and drug delivery vehicles.

For example, many viral-based expression vectors have been deployed for expression of heterologous proteins in cultured recombinant cells. For example, the application of modified viral vectors for gene expression in host cells continues to expand. Recent advances in this regard include further development of techniques and systems for production of multi-subunit protein complexes, and co-expression of protein-modifying enzymes to improve heterologous protein production. Other recent progresses regarding viral expression vector technologies include many advanced genome engineering applications for controlling gene expression, preparation of viral vectors, in vivo gene therapy applications, and creation of vaccine delivery vectors.

However, there is still a need for more efficient methods and systems for expressing products of interest in RNA replicon-based expression platforms.

### SUMMARY

The present disclosure relates generally to the development of immuno-therapeutics, such as recombinant nucleic acids constructs and pharmaceutical compositions including the same for use in the prevention and management of various health conditions such as proliferative disorders and microbial infection. In particular, as described in greater detail below, some embodiments of the disclosure provide nucleic acid constructs containing sequences that encode a modified genome or replicon of the alphavirus wherein a substantial portion of the nucleic acid sequence encoding the viral structural proteins of the modified alphavirus genome or replicon RNA is replaced by a synthetic adaptor molecule configured for facilitating insertion of a heterologous sequence into the sequence encoding the modified alphavirus genome or replicon RNA. Also disclosed are nucleic acid constructs containing sequences that encode a modified alphavirus genome or replicon RNA wherein there is a restriction enzyme site inserted after the poly(A) sequence for creating a DNA template that results in the 3′ terminus of the replicon RNA to contain only adenylate residues. Without being bound to any particular theory, alphavirus replicon RNAs containing only adenylate residues are believed to enhance the biological activity of the replicon RNAs. Also disclosed are recombinant cells and transgenic animals that have been engineered to include one or more of the nucleic acid constructs disclosed herein, methods for producing a molecule of interest, as well as pharmaceutical compositions. Further provided in particular aspects of the disclosure are compositions and methods for modulating an immune response in a subject in need thereof, and/or for the prevention and/or treatment of various health conditions, including proliferative disorders (e.g., cancers) and chronic infections.

In one aspect of the disclosure, provided herein are nucleic acid constructs including a modified alphavirus genome or replicon RNA, wherein a substantial portion of the nucleic acid sequence encoding the viral structural proteins of the modified alphavirus genome or replicon RNA is replaced by a synthetic adaptor molecule configured for facilitating insertion of a heterologous sequence into the modified alphavirus genome or replicon RNA, and wherein the synthetic adaptor molecule having the Formula I:

[5′ flanking domain]−[restriction site]n−/[3′ flanking domain] Formula I
wherein a) n is an integer from 1 to 6;
b) the restriction site is cleavable by a restriction endonuclease; and
c) the 5′ flanking domain and 3′ flanking domain each include a nucleic acid sequence predicted to have minimal secondary structure.

Non-limiting exemplary embodiments of the nucleic acid constructs of the disclosure can include one or more of the following features. In some embodiments, the 5′ flanking domain does not include a sequence which encodes an RNA sequence capable of forming a stem-loop structure. In some embodiments, the sequences of the 5′ flanking domain has a folding 40 value of the minimum free energy (MFE) structure higher than a predefined threshold value. In some embodiments, the 5′ flanking domain includes a coding sequence for an autoproteolytic peptide. In some embodiments, the coding sequence for the autoproteolytic peptide is incorporated upstream of the restriction site(s). In some embodiments, the autoproteolytic peptide includes one or more autoproteolytic cleavage sequences derived from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof. In some embodiments, the coding sequence for the autoproteolytic peptide is incorporated upstream of the restriction site(s). In some embodiments, the 5′ flanking domain includes an internal ribosomal entry site (IRES).

In some embodiments, the 5′ flanking domain does not include a translation start site in any reading frame. In some embodiments, the 5′ flanking domain includes a translation start site or a part thereof as the last nucleotides of the 5′ adaptor sequence. In some embodiments, the 5′ flanking domain includes a methionine codon as the last three nucleotides of the 5′ adaptor sequence. In some embodiments, the 5′ flanking domain has a length of from about 15 nucleotides to about 35 nucleotides. In some embodiments, 5′ flanking domain has a length of about 30 nucleotides. In some embodiments, 5′ flanking domain includes a nucleic acid sequence having at least 70%, at least 80% at least 90%, or at least 95% sequence identity to the sequence of SEQ ID NO: 1.

In some embodiments, the sequences of the 3′ flanking domain has a folding ΔG value of the minimum free energy (MFE) structure higher than a predefined threshold value. In some embodiments, the 3′ flanking domain does not include a sequence which encodes an RNA sequence capable of forming a stem-loop structure. In some embodiments, the 3′ flanking domain include a translation stop codon as the first three nucleotides of the 3′ adaptor sequence. In some embodiments, the stop codon is selected from TAG, TAA, or TGA. In some embodiments, the 3′ flanking domain includes a nucleic acid sequence having at least 70%, at least 80% at least 90%, or at least 95% sequence identity to SEQ ID NO: 2.

In some embodiments, the 5′ flanking domain of the synthetic adaptor molecule does not encode for an RNA sequence capable of forming a stem-loop structure with a sequence located immediately upstream thereof (e.g., in the sgRNA 5′ UTR) or with a sequence located immediately downstream thereof (e.g., within the coding sequence of a GOI). In some embodiments, the 3′ flanking domain does not encode for an RNA sequence capable of forming a stem-loop structure with a sequence located immediately upstream thereof (e.g., within the coding sequence of a GOI) or with a sequence located immediately downstream (e.g., in the 3′ UTR). In some embodiments, the 5′ flanking domain and/or 3′ flanking domain does not include a sequence having complementarity with a sequence located within the 3′ UTR. In some embodiments, the 5′ flanking domain and/or 3′ flanking domain does not include a sequence having complementarity with the 3′ end of the 3′ UTR.

In some embodiments, the synthetic adaptor molecule includes a nucleic acid sequence having at least 70%, at least 80% at least 90%, or at least 95% sequence identity to SEQ ID NO: 20.

In some embodiments, the restriction site is cleavable by a restriction enzyme selected from Type I restriction enzymes, Type II restriction enzymes, Type III restriction enzymes, Type IV restriction enzymes, and Type V restriction enzymes. In some embodiments, the restriction site is cleavable by a Type II restriction enzyme. In some embodiments, the restriction site is cleavable by SpeI or an isoschizomer thereof. In some embodiments, the isoschizomer of SpeI is AhII, BcuI, or SpeI-HF.

In some embodiments, the nucleic acid constructs of the disclosure further include an additional restriction site incorporated into the sequence encoding the poly(A) tail of the modified alphavirus genome or replicon RNA. In some embodiments, the additional restriction site is incorporated at the end of the sequence encoding the poly(A) tail of the alphavirus genome or replicon RNA. In some embodiments, the additional restriction site is cleavable by a Type IIS restriction enzyme or a homing endonuclease. In some embodiments, the Type IIS restriction enzyme is AcuI, AlwI, Alw26I, BaeI, BbiI, BbsI, BbsI-HF, BbvI, BccI, BceAI, BcgI, BciVI, BcoDI, BfuAI, BmrI, BpmI, BpuEI, BsaI, BsaI-HF, BsaI-HFv2, BsaXI, BseGI, BseRI, BsgI, BsmAI, BsmBI-v2, BsmFI, BsmI, BspCNI, BspMI, BspQI, BsrDI, BsrI, BtgZI, BtsCI, BtsI-v2, BtsIMutI, CspCI, EarI, EciI, Eco31I, Esp3I, FauI, FokI, HgaI, HphI, HpyAV, LpuI, MboII, MlyI, Mmel, MnlI, NmeAIII, PaqCI, PleI, SapI, or SfaNI. In some embodiments, the additional restriction site is cleavable by SapI or an isoschizomer thereof. In some embodiments, the isoschizomer of SapI is LguI, PciSI, or BspQI. In some embodiments, the additional restriction site is cleavable by a homing endonuclease. In some embodiments the homing endonuclease is I-CeuI, I-SceI, PI-PspI, PI-SceI.

In some embodiments, the nucleic acid constructs of the disclosure include a lengthened sequence encoding a poly(A) tail that is longer than the 11 residues previously considered to be sufficient for efficient minus strand synthesis. In some embodiments, the lengthened poly(A) tail is longer than 34 residues, which previously has not been observed to further enhance replication compared to a poly(A) tail of 25 residues. In some embodiments, the lengthened poly(A) tail has a length ranging from about 30 to about 120 adenylate residues. In some embodiments, the lengthened poly(A) tail has a length of about 120 adenylate residues. In some embodiments, the lengthened poly(A) tail has a length of about 30, about 40, about 50, about 60, about 70, about 80, about 90, and about 100 adenylate residues.

In some embodiments, the modified genome or replicon RNA is of a virus belonging to the Alphavirus genus of the Togaviridae family. In some embodiments, the modified genome or replicon RNA is of an alphavirus belonging to the VEEV/EEEV group, or the SFV group, or the SINV group. In some embodiments, the alphavirus is Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), or Buggy Creek virus. In some embodiments, the alphavirus is Venezuelan equine encephalitis virus (VEEV), Eastern Equine Encephalitis virus (EEEV), Chikungunya virus (CHIKV), or Sindbis virus (SINV).

In some embodiments, the nucleic acid constructs of the disclosure further include one or more expression cassettes, wherein each of the expression cassettes includes a promoter operably linked to a heterologous nucleic acid sequence. In some embodiments, at least one of the expression cassettes includes a subgenomic (sg) promoter operably linked to a heterologous nucleic acid sequence. In some embodiments, the sg promoter is a 26S subgenomic promoter. In some embodiments, the nucleic acid constructs of the disclosure further include one or more untranslated regions (UTRs). In some embodiments, at least one of the UTRs is a heterologous UTR.

In some embodiments, at least one of expression cassettes includes a coding sequence for a gene of interest (GOI). In some embodiments, the GOI coding sequence includes a stop codon positioned upstream of the 3′ flanking domain of the synthetic adaptor molecule. In some embodiments, the GOI encodes a polypeptide selected from the group consisting of a therapeutic polypeptide, a prophylactic polypeptide, a diagnostic polypeptide, a nutraceutical polypeptide, an industrial enzyme, and a reporter polypeptide. In some embodiments, the GOI encodes a polypeptide selected from the group consisting of an antibody, an antigen, an immune modulator, an enzyme, a signaling protein, and a cytokine. In some embodiments, the coding sequence of the GOI is optimized for expression at a level higher than the expression level of a reference coding sequence. In some embodiments, the coding sequence of the GOI does not contain restriction enzyme site(s) that are used to linearize the nucleic acid construct encoding the modified alphavirus genome or replicon RNA. In some embodiments, the nucleic acid construct is incorporated within a vector. In some embodiments, the vector is a self-replicating RNA (srRNA) vector. In some embodiments, the nucleic acid sequence has at least 70%, at least 75%, at least 80%, at least 85%, at least 90%, at least 95%, at least 96%, at least 97%, at least 98%, at least 99%, or 100% sequence identity to a nucleic acid sequence selected from the group consisting of SEQ ID NOS: 3-27.

In one aspect, provided herein are recombinant cells including a nucleic acid construct as described herein. In a related aspect, provided herein are cell cultures including at least one recombinant cell as described herein and a culture medium. Non-limiting exemplary embodiments of the recombinant cells of the disclosure can include one or more of the following features. In some embodiments, the recombinant cell is a prokaryotic cell or a eukaryotic cell. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the recombinant cell is a mammalian cell. In some embodiments, the recombinant cell is selected from the group consisting of African green monkey kidney cell (Vero cell), baby hamster kidney (BHK) cell, Chinese hamster ovary cell (CHO cell), human A549 cell, human cervix cell, human CHME5 cell, human epidermoid larynx cell, human fibroblast cell, human HEK-293 cell, human HeLa cell, human HepG2 cell, human HUH-7 cell, human MRC-5 cell, human muscle cell, mouse 3T3 cell, mouse connective tissue cell, mouse muscle cell, and rabbit kidney cell.

In another aspect, provided herein are transgenic animals including a nucleic acid construct as described herein. In some embodiments, the transgenic animal is a vertebrate animal or an invertebrate animal. In some embodiments, the transgenic animal is a mammalian. In some embodiments, the transgenic mammalian is a non-human mammalian.

In another aspect, provided herein are methods for producing a recombinant RNA molecule, the methods include (i) rearing a transgenic animal as described herein, or (ii) culturing a recombinant cell as described herein under conditions such that the recombinant RNA molecule is produced by the transgenic animal or in the recombinant cell. In some embodiments, the transgenic animal or the recombinant cell including a nucleic acid construct as described herein and wherein the sequence encoding the recombinant RNA molecule is optionally digested by a restriction enzyme capable of cleaving the restriction site engineered after the end of the sequence encoding the poly(A) tail to generate a template that encodes for an RNA that only has adenylate residues in the poly(A) tail and 3′ terminus. Accordingly, recombinant RNA molecules produced according to a method described herein are also provided by the present disclosure. In some embodiments, the recombinant RNA molecules described herein exhibit enhanced biologic activity.

In another aspect, provided herein are methods for producing a polypeptide of interest, the methods include (i) rearing a transgenic animal comprising a nucleic acid construct as described herein, or (ii) culturing a recombinant cell including a nucleic acid construct as described herein under conditions wherein the polypeptide encoded by the GOI is produced by the transgenic animal or in the recombinant cell. In another aspect, provided herein are methods for producing a polypeptide of interest, the methods include administering to the subject a nucleic acid construct described herein. Non-limiting exemplary embodiments of the methods of the disclosure can include one or more of the following features. In some embodiments, the subject is vertebrate animal or an invertebrate animal. In some embodiments, the subject is a mammalian subject. In some embodiments, the mammalian subject is a human subject. Accordingly, recombinant polypeptides produced according to a method described herein are also provided by the present disclosure.

In one aspect, provided herein are pharmaceutical compositions including a pharmaceutically acceptable excipient and one or more of the following: (a) a nucleic acid construct described herein; (b) a recombinant RNA molecule as described herein; (c) a recombinant cell as described herein; and (d) a recombinant polypeptide as described herein.

Non-limiting exemplary embodiments of the pharmaceutical compositions of the disclosure can include one or more of the following features. In some embodiments, the pharmaceutical compositions include a nucleic acid construct as described herein, and a pharmaceutically acceptable excipient. In some embodiments, the pharmaceutical compositions include a recombinant cell as described herein, and a pharmaceutically acceptable excipient. In some embodiments, the pharmaceutical compositions include a recombinant RNA molecule as described herein, and a pharmaceutically acceptable excipient. In some embodiments, the pharmaceutical compositions include a recombinant polypeptide as described herein, and a pharmaceutically acceptable excipient. In some embodiments, the composition is formulated in a liposome, a lipid-based nanoparticle (LNP), or a polymer nanoparticle. In some embodiments, the composition is an immunogenic composition. In some embodiments, immunogenic composition is formulated as a vaccine. In some embodiments, immunogenic composition is formulated as a biotherapeutic, e.g., vehicle for gene delivery of different molecules with bioactivity. In some embodiments, the composition is substantially non-immunogenic to a subject. In some embodiments, non-immunogenic composition is formulated as a vaccine. In some embodiments, non-immunogenic composition is formulated as a biotherapeutic. In some embodiments, the pharmaceutical composition is formulated as an adjuvant. In some embodiments, the pharmaceutical composition is formulated for one or more of intranasal administration, transdermal administration, intraperitoneal administration, intramuscular administration, intranodal administration, intratumoral administration, intraarticular administration, intravenous administration, subcutaneous administration, intravaginal, and oral administration.

In another aspect, provided herein are methods for modulating an immune response in a subject in need thereof, the methods include administering to the subject a composition including one or more of the following: (a) a nucleic acid construct as described herein; (b) a recombinant RNA molecule as described herein; (c) a recombinant cell as described herein; (d) a recombinant polypeptide as described herein; and (e) a pharmaceutical composition as described herein.

In another aspect, provided herein are methods for preventing and/or treating a health condition in a subject in need thereof, the methods include prophylactically or therapeutically administering to the subject a composition including one or more of the following: (a) a nucleic acid construct as described herein; (b) a recombinant RNA molecule as described herein; (c) a recombinant cell as described herein; (d) a recombinant polypeptide as described herein; and (e) a pharmaceutical composition as described herein.

Implementations of embodiments of the methods of preventing, and/or ameliorating, and/or treating a health condition according to the present disclosure can include one or more of the following features. In some embodiments, the health condition is a proliferative disorder, inflammatory disorder, autoimmune disorder, or a microbial infection. In some embodiments, the subject has or is suspected of having a condition associated with proliferative disorder, inflammatory disorder, autoimmune disorder, or a microbial infection. In some embodiments, the subject has or is suspected of having a condition associated with a rare disease. In some embodiments, the composition is administered to the subject individually as a single therapy (monotherapy) or as a first therapy in combination with at least one additional therapies. In some embodiments, the at least one additional therapies is selected from the group consisting of chemotherapy, radiotherapy, immunotherapy, hormonal therapy, toxin therapy, targeted therapy, and surgery.

In yet another aspect, provided herein are kits for modulating an immune response, for the prevention, and/or for the treatment of a health condition or a microbial infection, the kits include one or more of the followings: (a) a nucleic acid construct of as described herein; (b) a recombinant RNA molecule as described herein; (c) a recombinant cell as described herein; (d) a recombinant polypeptide as described herein; and (e) a pharmaceutical composition as described herein.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1B are schematic representations of non-limiting examples of the alphavirus vector designs in accordance with some embodiments of the disclosure, in which the coding sequence of the viral structural proteins of the original alphavirus has been deleted and a synthetic adaptor molecule has been inserted upstream of the 3′ UTR. FIG. 1A illustrates a non-limiting example of an exemplary modified alphavirus vector design in accordance with some embodiments of the disclosure. In this example, the synthetic adaptor molecule contains, in 5′→3′ direction, a 5′ flanking domain, a SpeI recognition restriction site, and a 3′ flanking domain. This modified alphavirus vector design (empty vector) also contains a 26S subgenomic promoter (26S) and 5′ UTR and 3′ UTR sequences and poly(A) tail. Non-structural proteins NSP1, NSP2, NSP3, and NSP4 are also shown. FIG. 1B depicts the structure of another alphavirus design derived from the vector described in FIG. 1A. In this design, a heterologous gene of interest (GOI) is cloned into the SpeI restriction site such that its expression is placed under control of the 26S subgenomic promoter.

FIGS. 2A-21 are graphical illustrations of four non-limiting exemplary alphavirus RNA replicon designs (empty vectors) in accordance with some embodiments of the disclosure, in which the sequences encoding a modified Venezuelan equine encephalitis (VEE) genome, a modified Chikungunya virus (CHIKV) strain 27, a modified CHIKV strain DRDE, a modified Eastern Equine Encephalitis virus (EEEV), a modified SINV strain Girdwood, or modified SINV strain AR86/Girdwood chimera genome, respectively, is incorporated into expression vectors, which also include a synthetic adaptor molecule inserted upstream of the respective 3′ UTR sequence.

FIGS. 3A-3F are graphical illustrations of five non-limiting exemplary alphavirus RNA replicon designs in accordance with some embodiments of the disclosure. In FIG. 3A, a modified Eastern Equine Encephalitis virus (EEEV) genome in accordance with some embodiments of the disclosure is incorporated into expression vector which also contains a coding sequence for an exemplary gene of interest (GOI), e.g., hemagglutinin precursor (HA) of the influenza A virus H5N1 inserted into a synthetic adaptor molecule. In FIGS. 3B-3F, a coding sequence for H5N1 HA is inserted in expression vectors containing a modified SINV AR86-Girdwood chimera designs in accordance with some embodiments of the disclosure.

FIG. 4 is a schematic representation of a non-limiting example of the alphavirus vector DNA template designs in accordance with some embodiments of the disclosure, in which a Type IIS restriction endonuclease recognition site has been added downstream of the poly(A). FIG. 4A illustrates a state-of-the art DNA template sequenced used for in vitro transcription of alphavirus vector RNA, in which RNA transcription is initiated at the site of a 5′ T7 promoter (T7 prom) and terminated by transcription into a T7 terminator (T7 term). FIG. 4B illustrates a non-limiting example of an exemplary modified alphavirus vector design in accordance with some embodiments of the disclosure. In this example, a SapI restriction endonuclease recognition site is inserted immediately downstream of the poly(A) sequence. Since SapI is a Type IIS restriction endonuclease that cleaves DNA outside of its recognition site (sequence shown in box), the digest product leaves only deoxythymidine residues on the 5′ terminus on the DNA template sequence which encode for adenosyl residues in the RNA product. In this example, when the DNA is linearized by SapI digestion and used as the template for in vitro transcription, RNA transcription is initiated at the site of the 5′ T7 promoter (T7 prom) and terminates by run-off transcription at the end of the poly(A), leaving only adenylate residues on the 3′ terminus of the RNA, in contrast to termination by the T7 promoter, which results in non-adenylate residues to be transcribed on the 3′ end of the RNA product.

FIG. 5 is a bar graph representing the difference between replicon RNA with a 3′ terminus that (i) consists of 30 adenylate residues (A) or (ii) consists of 30 adenylate residues followed by the transcribed terminator (T7) sequence. Different amounts of the replicon RNAs were electroporated into BHK-21 cells in triplicate and 17.5 hours later the resulting frequency of cells containing dsRNA as a result of replicon replication or expression of the encoded transgene of interest (HA) was quantified by fluorescence flow cytometry. At this timepoint, at sub-saturating amounts (<250 ng) of transfected replicon RNA, there is evidence of enhanced biologic activity in the form of significantly higher replication and transgene expression for the replicon RNA with the 3′ terminus that ends in adenylate residues versus ending with the T7 terminator sequence.

FIG. 6 is a bar graph representing the difference between replicon RNA with a 3′ terminus that consists of 30 adenylate residues followed by the transcribed terminator (30; T7) sequence, or consists of 30 adenylate residues (30; Clean) or approximately 120 adenylate residues (˜120; Clean). Either 25 or 100 ng of replicon RNA was electroporated into BHK-21 cells in duplicate and 20 hours later the resulting frequency of cells containing dsRNA as a result of replicon replication or expression of the encoded transgene of interest (HA) was quantified by fluorescence flow cytometry. In this example, the replicon RNA with the lengthened poly(A) tail exhibits enhanced biologic activity in the form of higher replication and transgene expression.

FIG. 7 schematically compares the recognition sequence and cleavage site of Type II versus Type IIS restriction enzymes.

FIG. 8 pictorially summarizes the results of electrophoresis analytical experiments performed to evaluate the integrity of srRNA molecules prepared by in vitro transcription (IVT) using a plasmid DNA template linearized by enzymatic digestion. In this example, the DNA was linearized with SapI which cuts at the end of the poly(A) sequence (e.g., cuts immediately downstream of the poly(A) sequence).

FIG. 9 schematically summarizes the results of experiments performed to illustrate specific differences in RNA replication activity of srRNAs in correlation with the length of their poly(A) tails. srRNA constructs in a range of doses were electroporated (EP) into cells, and the frequency of RNA replication was quantified by detection of double stranded RNA (dsRNA) by using flow cytometry.

FIG. 10 schematically summarizes the quantitative differences of RNA replication activity of srRNAs in correlation with the length of their poly(A) tails. The inverse of the EC50 (RNA dose for half-maximal activity) was calculated from fitting the data shown in FIG. 9 to a 4PL curve, and a one-way ANOVA statistical test was performed to determine significance between the Log(EC50) values.

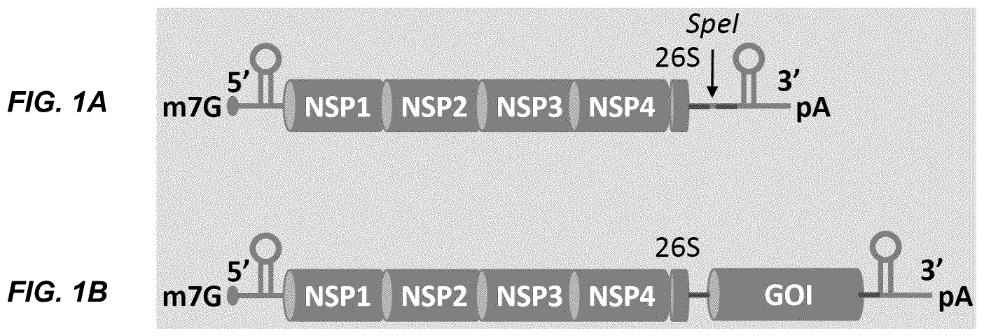

### DETAILED DESCRIPTION OF THE DISCLOSURE

Provided herein are, inter alia, viral expression systems with superior expression potential which are suitable for expressing heterologous molecules such as, for example, vaccines and therapeutic polypeptides, in recombinant cells. For example, some embodiments of the disclosure relate to nucleic acid constructs such as, e.g. expression constructs and vectors, containing a modified genome or replicon RNA of an alphavirus in which a substantial portion of its original viral sequence encoding structural proteins has been deleted. Also provided in some embodiments of the disclosure are viral-based expression vectors including one or more expression cassettes encoding heterologous polypeptide. Further provided in some embodiments of the disclosure are nucleic acid constructs such as, e.g. expression constructs and vectors, containing a modified genome or replicon RNA of Eastern Equine Encephalitis Virus (EEEV) or Sindbis viruses (SINV) in which at least some of its original viral sequence encoding structural proteins has been deleted. Further provided are recombinant cells that are genetically engineered to include one or more of the nucleic acid molecules disclosed herein. Biomaterials and recombinant products derived from such recombinant cells are also within the scope of the application. Also provided are compositions and methods useful for modulating an immune response in a subject in need thereof, as well as methods for preventing and/or treating various health conditions.

Self-amplifying RNAs (replicons) based on RNA viruses (e.g., alphaviruses) can be used as robust expression systems. For example, it has been reported that a non-limiting advantage of using alphaviruses such as EEEV and SINV as viral expression vectors is that they can direct the synthesis of large amounts of heterologous proteins in recombinant host cells. In particular, the alphavirus replicon platform systems disclosed herein are capable of expressing high levels of heterologous polypeptides of interest. Among other advantages, polypeptides such as therapeutic single chain antibodies may be most effective if expressed at high levels in vivo. In addition, for producing recombinant antibodies purified from cells in culture (ex vivo), high protein expression from a replicon RNA may increase overall yields of the antibody product. Furthermore, if the protein being expressed is a vaccine antigen, high level expression may induce the most robust immune response in vivo.

Alphaviruses utilize motifs contained in their UTRs, structural regions, and non-structural regions to impact their replication in host cells. These regions also contain mechanisms to evade host cell innate immunity. There can often be significant differences between Alphaviruses. Which part of the genome contains these functional components also varies between Alphaviruses. Beyond variation between individual Alphaviruses, there are often differences within strains of Alphaviruses as well that can account for changes in characteristics such as virulence. For example, sequence variations between North American and South American strains of EEEV alter the ability to modulate the STAT1 pathway leading to differential induction of Type I interferons and resulting changes in virulence. As described below, some embodiments of the disclosure relate to modified alphavirus genomes or replicon RNAs based on EEEV. As a further example, SINV strain S.A.AR86 (AR86) rapidly and robustly inhibits tyrosine phosphorylation of STAT1 and STAT2 in response to IFN-γ and/or IFN-β, but related SINV strain Girdwood is an inefficient inhibitor of STAT1/2 activation. A unique threonine at position 538 in the non-structural protein of AR86 results in slower non-structural protein processing and delayed subgenomic RNA synthesis from the related SINV strain Girdwood, which contributes to an adult mouse neurovirulence phenotype and could be advantageous for the kinetics and yield of heterologous protein expression and contribute to a more robust immune response to a vaccine antigen expressed from AR86-based replicon vectors. A true AR86 replicon that contains the T538 has not been described. As described in greater detail below, a functional AR86 replicon using the reported genome sequence (Genbank U38305) could not be created, which is presumably why existing AR86-based replicons carry the attenuating T538I mutation. However, it was found that one can generate functional AR86 replicons that still bear T538 by creating specific chimeras with the nsP genes from Girdwood. As further described below, some embodiments of the disclosure relate to modified alphavirus genomes or replicon RNAs based on SINV strain AR86.

As described in greater detail below, some embodiments of the disclosure relate to modified alphavirus genomes or replicon RNAs that have been engineered to incorporate a restriction site at the end of the sequence encoding the poly(A) tail to provide enhanced biologic activity such as, increased level of replication, expression, and/or translation.

Also described in greater detail below, some embodiments of the disclosure relate to modified alphavirus genomes or replicon RNAs that have been engineered to have lengthened poly(A) tails to provide enhanced biologic activity such as, increased level of replication, expression, and/or translation.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The terms “administration” and “administering”, as used herein, refer to the delivery of a bioactive composition or formulation by an administration route comprising, but not limited to, intranasal, transdermal, intravenous, intra-arterial, intramuscular, intranodal, intraperitoneal, subcutaneous, intramuscular, oral, intravaginal, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “construct” refers to a recombinant molecule including one or more isolated nucleic acid sequences from heterologous sources. For example, nucleic acid constructs can be chimeric nucleic acid molecules in which two or more nucleic acid sequences of different origin are assembled into a single nucleic acid molecule. Thus, representative nucleic acid constructs include any constructs that contain (1) nucleic acid sequences, including regulatory and coding sequences that are not found adjoined to one another in nature (e.g., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative nucleic acid constructs can include any recombinant nucleic acid molecules, linear or circular, single-stranded or double-stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. Constructs of the present disclosure can include the necessary elements to direct expression of a nucleic acid sequence of interest that is also contained in the construct. Such elements may include control elements such as a promoter that is operably linked to (so as to direct transcription of) the nucleic acid sequence of interest, and optionally includes a poly(A)denylation sequence.

In some embodiments of the disclosure, the nucleic acid construct may be incorporated within a vector. The term “vector” is used herein to refer to a nucleic acid molecule or sequence capable of transferring or transporting another nucleic acid molecule. Thus, the term “vector” encompasses both DNA-based vectors and RNA-base vectors. The term “vector” includes cloning vectors and expression vectors, as well as viral vectors and integrating vectors. An “expression vector” is a vector that includes a regulatory region, thereby capable of expressing DNA sequences and fragments in vitro, ex vivo, and/or in vivo. In some embodiments, a vector may include sequences that direct autonomous replication in a cell such as, for example a plasmid (DNA-based vector) or a self-replicating RNA vector. In some embodiments, a vector may include sequences sufficient to allow integration into host cell DNA. Useful vectors include, for example, plasmids (e.g., DNA plasmids or RNA plasmids), transposons, cosmids, bacterial artificial chromosomes, and viral vectors. In some embodiments, the vector of the disclosure can be single-stranded vector (e.g., ssDNA or ssRNA). In some embodiments, the vector of the disclosure can be double-stranded vector (e.g., dsDNA or dsRNA). In some embodiments, a vector is a gene delivery vector. In some embodiments, a vector is used as a gene delivery vehicle to transfer a gene into a cell.

In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a cell. Two or more constructs can be incorporated within a single nucleic acid molecule, such as a single vector, or can be incorporated within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in a cell. For the practice of the present disclosure, compositions and methods for preparing and using constructs and cells are known to one skilled in the art.

The term “effective amount”, “therapeutically effective amount”, or “pharmaceutically effective amount” of a composition of the disclosure, e.g., nucleic acid constructs (e.g., poly(A)vectors or srRNA molecules), recombinant cells, recombinant polypeptides, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, the term “replicon RNA” refers to RNA which contains all of the genetic information required for directing its own amplification or self-replication within a permissive cell. Therefore, replicon RNA is sometimes also referred to as “self-amplifying RNA” (saRNA) or “self-replicating RNA” (srRNA). To direct its own replication, the RNA molecule 1) encodes polymerase, replicase, or other proteins which may interact with viral or host cell-derived proteins, nucleic acids or ribonucleoproteins to catalyze the RNA amplification process; and 2) contain cis-acting RNA sequences required for replication and transcription of the subgenomic replicon-encoded RNA. These sequences may be bound during the process of replication to its self-encoded proteins, or non-self-encoded cell-derived proteins, nucleic acids or ribonucleoproteins, or complexes between any of these components. In some embodiments of the present disclosure, an alphavirus replicon RNA molecule (e.g., srRNA or saRNA molecule) generally contains the following ordered elements: 5′ viral or defective-interfering RNA sequence(s) required in cis for replication, sequences coding for biologically active alphavirus non-structural proteins (e.g., nsP1, nsP2, nsP3, and nsP4), promoter for the subgenomic RNA (sgRNA), 3′ viral sequences required in cis for replication, and a poly(A)denylate tract (poly(A)). In some instances, a subgenomic promoter (sg) that directs expression of a heterologous sequence can be included in the srRNA construct of the disclosure. Further, the term replicon RNA (e.g., srRNA or saRNA) generally refers to a molecule of positive polarity, or “message” sense, and the replicon RNA may be of length different from that of any known, naturally-occurring alphavirus. In some embodiments of the present disclosure, the replicon RNA does not contain the sequences of at least one of structural viral protein; sequences encoding structural genes can be substituted with heterologous sequences. In those instances, where the replicon RNA is to be packaged into a recombinant alphavirus particle, it can contain one or more sequences, so-called packaging signals, which serve to initiate interactions with alphavirus structural proteins that lead to particle formation.

As used herein, a “subject” or an “individual” includes animals, such as human (e.g., human subject) and non-human animals. In some embodiments, a “subject” or “individual” is a patient under the care of a physician. Thus, the subject can be a human patient or a subject who has, is at risk of having, or is suspected of having a disease of interest (e.g., cancer) and/or one or more symptoms of the disease. The subject can also be a subject who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about” which, as used herein, has its ordinary meaning of approximately. The term “about” is used to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein may be contiguous or non-contiguous (e.g., linked to one another through a linker).

The term “portion” as used herein refers to a fraction. With respect to a particular structure such as a polynucleotide sequence or an amino acid sequence or protein the term “portion” thereof may designate a continuous or a discontinuous fraction of said structure. For example, a portion of an amino acid sequence comprises at least 1%, at least 5%, at least 10%, at least 20%, at least 30%, at least 40%, at least 50%, at least 60%, at least 70%, at least 80%, and at least 90% of the amino acids of said amino acid sequence. In addition or alternatively, if the portion is a discontinuous fraction, said discontinuous fraction is composed of 2, 3, 4, 5, 6, 7, 8, or more parts of a structure (e.g., domains of a protein), each part being a continuous element of the structure. For example, a discontinuous fraction of an amino acid sequence may be composed of 2, 3, 4, 5, 6, 7, 8, or more, for example not more than 4 parts of said amino acid sequence, wherein each part comprises at least 1, at least 2, at least 3, at least 4, at least 5 continuous amino acids, at least 10 continuous amino acids, at least 20 continuous amino acids, or at least 30 continuous amino acids of the amino acid sequence.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region as measured using a BLAST or BLAST 2.0 sequence comparison algorithms with default parameters described below, or by manual alignment and visual inspection. See, e.g., the NCBI web site at ncbi.nlm.nih.gov/BLAST. This definition also refers to, or may be applied to, the complement of a query sequence. This definition includes sequence comparison performed by a BLAST algorithm wherein the parameters of the algorithm are selected to give the largest match between the respective sequences over the entire length of the respective reference sequences. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al., Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Additional methodologies that can suitably be utilized to determine similarity or identity amino acid sequences include those relying on position-specific structure-scoring matrix (P3SM) that incorporates structure-prediction scores from Rosetta, as well as those based on a length-normalized edit distance as described previously in, e.g., Setcliff et al., Cell Host & Microbe 23(6), May 2018.

The term “pharmaceutically acceptable excipient” as used herein refers to any suitable substance that provides a pharmaceutically acceptable carrier, additive, or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a health condition of interest (e.g., cancer or infection) and/or one or more symptoms of the health condition. The subject can also be an individual who is diagnosed with a risk of the health condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Where a range of values is provided, it is understood by one having ordinary skill in the art that all ranges disclosed herein encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Alphaviruses

Alphavirus is a genus of genetically, structurally, and serologically related viruses of the group IV Togaviridae family which includes at least 30 members, each having single stranded RNA genomes of positive polarity enclosed in a nucleocapsid surrounded by an envelope containing viral spike proteins. Currently, the alphavirus genus comprises among others the Sindbis virus (SIN), the Semliki Forest virus (SFV), the Ross River virus (RRV), Venezuelan equine encephalitis virus (VEEV), and Eastern equine encephalitis virus (EEEV), which are all closely related and are able to infect various vertebrates such as mammalians, rodents, fish, avian species, and larger mammals such as humans and horses as well as invertebrates such as insects. Transmission between species and individuals occurs mainly via mosquitoes making the alphaviruses a contributor to the collection of Arboviruses—or Arthropod-Borne Viruses. In particular, the Sindbis and the Semliki Forest viruses have been widely studied and the life cycle, mode of replication, etc., of these viruses are well characterized. In particular, alphaviruses have been shown to replicate very efficiently in animal cells which makes them valuable as vectors for production of protein and nucleic acids in such cells.

Each of these alphaviruses has a single stranded RNA genome of positive polarity enclosed in a nucleocapsid surrounded by an envelope containing viral spike proteins. Alphavirus particles are enveloped, tend to be spherical (although slightly pleomorphic), and have an isometric nucleocapsid. Alphavirus genome is single-stranded RNA of positive polarity of approximately 11-12 kb in length, comprising a 5′ cap, a 3′ poly-A tail, and two open reading frames with a first frame encoding the non-structural proteins with enzymatic function and a second frame encoding the viral structural proteins (e.g., the capsid protein CP, E1 glycoprotein, E2 glycoprotein, E3 protein and 6K protein).

The 5′ two-thirds of the alphavirus genome encodes a number of non-structural proteins (nsPs) necessary for transcription and replication of viral RNA. These proteins are translated directly from the RNA and together with cellular proteins form the RNA-dependent RNA polymerase essential for viral genome replication and transcription of sgRNA. Four nsPs (nsP1-4) are produced as a single polyprotein constitute the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises sgRNA which serves as a template for translation of all the structural proteins required for forming viral particles: the core nucleocapsid protein C, and the envelope proteins P62 and E1 that associate as a heterodimer. The viral membrane-anchored surface glycoproteins are responsible for receptor recognition and entry into target cells through membrane fusion. The sgRNA is transcribed from the p26S subgenomic promoter present at the 3′ end of the RNA sequence encoding the nsp4 protein. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphaviral glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses lose the peripheral protein E3, while in Semliki viruses it remains associated with the viral surface.

Alphavirus replication has been reported to take place on membranous surfaces within the host cell. In the first step of the infectious cycle, the 5′ end of the genomic RNA is translated into a polyprotein (nsP1-4) with RNA polymerase activity that produces a negative strand complementary to the genomic RNA. The sequence at the 3′ end of the genomic RNA plays an important role in the initiation negative-strand synthesis, where a minimum number of adenylate residues has been identified to be essential for replication to occur. In particular, it has been previously reported that for alphavirus genomes to replicate, there must be at least 11 residues in the poly(A) tail following the 3′ UTR to efficiently initiate minus-strand synthesis, and therefore replication to occur. It has also been previously reported that lengthening the poly(A) tail to 25 residues results in enhanced replication, but no further enhancement of replication was observed when the poly(A) was lengthened further to 34 residues. In addition, internal non-A residues in the poly(A) are most often deleterious to replication, which suggests that enzymatic poly(A) tailing would not benefit replicon RNA that did not exclusively contain 3′ adenylate residues following the 3′ UTR. It has been previous reported that there is no enhancement of minus-strand synthesis on RNA templates with greater than 25 adenylate residues in the poly(A) tail. In a second step of replication, the negative strand is used as a template for the production of two RNAs, respectively: (1) a positive genomic RNA corresponding to the genome of the secondary viruses producing, by translation, other nsPs and acting as a genome for the virus; and (2) sgRNA encoding the structural proteins of the virus forming the infectious particles. The positive genomic RNA/sgRNA ratio is regulated by proteolytic autocleavage of the polyprotein to nsP1, nsP2, nsP3 and nsP4. In practice, the viral gene expression takes place in two phases. In a first phase, there is main synthesis of positive genomic strands and of negative strands. During the second phase, the synthesis of sgRNA is virtually exclusive, thus resulting in the production of large amount of structural protein.

As described above, there can often be significant differences between Alphaviruses. Which parts of the genome that contain components with different or synonymous functions also varies between Alphaviruses. Beyond variation between individual Alphaviruses, there are often differences within strains of Alphaviruses as well that can account for changes in characteristics such as virulence. For example, sequence variations between North American and South American strains of EEEV alter the ability to modulate the STAT1 pathway leading to differential induction of Type I interferons and resulting changes in virulence. As described below, some embodiments of the disclosure relate to modified alphavirus genomes or replicon RNAs based on EEEV. As a further example, SINV strain S.A.AR86 (AR86) rapidly and robustly inhibits tyrosine phosphorylation of STAT1 and STAT2 in response to IFN-γ and/or IFN-β, but related SINV strain Girdwood is an inefficient inhibitor of STAT1/2 activation. A unique threonine at position 538 in the non-structural protein of AR86 results in slower non-structural protein processing and delayed subgenomic RNA synthesis from the related SINV strain Girdwood, which contributes to an adult mouse neurovirulence phenotype and could be advantageous for the kinetics and yield of heterologous protein expression and contribute to a more robust immune response to a vaccine antigen expressed from AR86-based replicon vectors. A functional AR86 replicon using the reported genome sequence (Genbank U38305) has not been created, likely due to the T538 phenotype described above, which is presumably why existing AR86-based replicons contain many alterations, including the attenuating T538I mutation. However, the experimental results presented herein have demonstrated that one can generate functional AR86 replicons that still bear T538 by creating specific chimeras with the nsP genes from Girdwood. As further described below, some embodiments of the disclosure relate to modified alphavirus genomes or replicon RNAs based on SINV strain AR86.

### Compositions of the Disclosure

As described in greater detail below, one aspect of the present disclosure relates to nucleic acid constructs a nucleic acid sequence encoding a modified alphavirus genome or replicon RNA, wherein at least a portion of the nucleic acid sequence encoding one or more structural proteins of the corresponding unmodified alphavirus genome or replicon RNA has been removed. Some embodiments of the disclosure provide a modified alphavirus genome or replicon RNA in which the coding sequence for non-structural proteins nsP1, nsP2, nsP3, and nsP4 is present, however at least a portion of or the entire sequence encoding one or more structural proteins is absent. Some embodiments of the disclosure provide a modified alphavirus genome or replicon RNA in which the coding sequence for non-structural proteins nsP1, nsP2, nsP3, and nsP4 is present, however a substantial portion of the sequence encoding structural proteins is absent. Also provided are recombinant cells and cell cultures that have been engineered to include a nucleic acid construct as disclosed herein.

### A. Nucleic Acid Constructs

As described in greater detail below, one aspect of the present disclosure relates to novel nucleic acid constructs including a nucleic acid sequence encoding a modified genome or replicon RNA of an alphavirus, such as Venezuelan equine encephalitis virus (VEEV), Eastern equine encephalitis virus (EEEV), Chikungunya virus (CHIKV) or Sindbis virus (SINV). For example, a modified alphavirus genome can include deletion(s), substitution(s), and/or insertion(s) in one or more of the genomic regions of the parent alphavirus genome.

Non-limiting exemplary embodiments of the nucleic acid constructs of the disclosure can include one or more of the following features. In some embodiments, the nucleic acid constructs include a modified alphavirus genome or replicon RNA, wherein a substantial portion of the nucleic acid sequence encoding the viral structural proteins of the modified alphavirus genome or replicon RNA is replaced by a synthetic adaptor molecule configured for facilitating insertion of a heterologous sequence into the modified alphavirus genome or replicon RNA. In some embodiments, the synthetic adaptor molecule having the Formula I:

[5′ flanking domain]−[restriction site]n−/[3′ flanking domain] Formula I
wherein a) n is an integer from 1 to 6;
b) the restriction site is cleavable by a restriction endonuclease; and
c) the 5′ flanking domain and 3′ flanking domain each include a nucleic acid sequence predicted to have minimal secondary structure.

In some embodiments, n is an integer from 1 to 6, such as for example, from 1 to 2, from 1 to 3, from 1 to 4, from 1 to 5, from 2 to 3, from 2 to 4, from 2 to 5, from 2 to 6, from 3 to 4, from 3 to 5, from 3 to 6, from 4 to 5, from 4 to 6, or from 5 to 6. In some embodiments, n is 1.

In some embodiments, the nucleic acid constructs include a nucleic acid sequence encoding a modified alphavirus genome or replicon RNA, wherein a substantial portion of the nucleic acid sequence encoding one or more structural proteins of the modified alphavirus genome or replicon RNA has been removed, e.g., the modified alphavirus genome or replicon RNA does not include at least a portion of the coding sequence for one or more of the alphavirus structural proteins CP, E1, E2, E3, and 6K.

Non-limiting exemplary embodiments of the nucleic acid constructs of the disclosure can include one or more of the following features. In some embodiments, at least a portion of the nucleic acid sequence encoding one or more of the viral structural proteins CP, E1, E2, E3, and 6K of the unmodified viral genome or replicon RNA has been removed. In some embodiments, a portion of or the entire sequence encoding CP has been removed. In some embodiments, a portion of or the entire sequence encoding E1 has been removed. In some embodiments, a portion of or the entire sequence encoding E2 has been removed. In some embodiments, a portion of or the entire sequence encoding E3 has been removed. In some embodiments, a portion of or the entire sequence encoding 6K has been removed. In some embodiments, a portion of or the entire sequence encoding a combination of CP, E1, E2, E3, and 6K has been removed. Some embodiments of the disclosure provide a modified alphavirus genome or replicon RNA in which the coding sequence for non-structural proteins nsP1, nsP2, nsP3, and nsP4 of the unmodified alphavirus genome or replicon RNA is present, however at least a portion of or the entire sequence encoding one or more structural proteins (e.g., CP, E1, E2, E3, and 6K) of the alphavirus genome or replicon RNA is absent. Some embodiments of the disclosure provide a modified alphavirus genome or replicon RNA in which a substantial portion of the nucleic acid sequence encoding structural proteins of the modified alphavirus genome or replicon RNA has been removed.

In some embodiments, a substantial portion of the nucleic acid sequence encoding one or more viral structural proteins has been removed. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full length nucleic acid sequence. As described above, the present disclosure provides nucleic acid molecules and constructs which are devoid of partial or complete nucleic acid sequences encoding one or more viral structural proteins. The skilled artisan, having the benefit of the sequences as disclosed herein, can readily use all or a substantial portion of the disclosed sequences for the compositions and methods of the disclosure. Accordingly, the present application comprises the complete sequences as disclosed herein, e.g., those set forth in the accompanying Sequence Listing, as well as substantial portions of those sequences as defined above.

In some embodiments, the entire sequence encoding viral structural proteins has been removed, e.g., the modified viral genome or replicon RNA includes no nucleic acid sequence encoding the structural proteins of the viral unmodified genome or replicon RNA.

The srRNA constructs of the disclosure generally have a length of at least about 2 kb. For example, the srRNA can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the srRNA can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the srRNA can have a length of about 6 kb to about 14 kb. In some embodiments, the srRNA can have a length of about 6 kb to about 16 kb.

### Synthetic Adaptor Molecule

As described above, the 5′ flanking domain and 3′ flanking domain of the synthetic adaptor molecule each include a nucleic acid sequence predicted to have minimal secondary structure, such as a stem-loop structure or hairpin structure which can potentially function as a polymerase termination signal, which in turn may cause premature termination. The skilled artisan will appreciate that the secondary structure of a nucleic acid sequence can be assessed by a variety of methodologies including those developed to determine or predict the folding ΔG value of a given nucleic acid sequence, or to determine the minimum free energy (MFE) structure of the nucleic acid sequence. Accordingly, in some embodiments, the sequences of the 5′ flanking domain of the synthetic adaptor molecule has a folding ΔG value of the MFE structure higher than a predefined threshold value. In some embodiments, the MFE structure of a nucleic acid sequence can be determined by using the Mfold tool for MFE RNA structure prediction and ΔG calculation based on that structure as described previously in, for example, Zuker M. Nucleic Acids Research, Volume 31, Issue 13, 1 Jul. 2003. Alternatively or in addition, the Vienna RNA Package publicly available at http://rna.tbi.univie.ac.at/ with a collection of commonly used programs for folding, design and analysis of RNA sequences can also be used. Accordingly, in some embodiments, the sequences of the 5′ flanking domain of the synthetic adaptor molecule has a folding ΔG value of the MFE structure greater than about >−9.6 kcal/mol for local hairpin/stem-loop structure. In some embodiments, the 5′ flanking domain does not include a sequence which encodes an RNA sequence capable of forming a stem-loop structure.

In some embodiments, the 5′ flanking domain includes a coding sequence for an autoproteolytic peptide, which can be useful in facilitating seamless and/or insulated expression of a protein of interest without N-terminal leader sequence. Suitable autoproteolytic peptides include, but are not limited to, autoproteolytic cleavage sequences derived from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A). In some embodiments, the coding sequence for the autoproteolytic peptide is incorporated upstream of the restriction site(s). For the purpose of this application, the term “upstream” in reference to a nucleic acid sequence designates a region located at the 5′ end of the nucleic acid sequence in question, and the term “downstream” designates a region located at the 3′ end of said nucleic acid sequence. Accordingly, in some embodiments, the 5′ flanking domain of the synthetic adaptor molecule includes a coding sequence for one or more autoproteolytic cleavage sequences derived from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

In some embodiments, the 5′ flanking domain includes an internal ribosomal entry site (IRES), which can be useful in facilitating insulated expression of a protein of interest. In some embodiments, the IRES element is incorporated upstream of the restriction site(s). IRES sequences suitable for the compositions and methods of the disclosure include, but are not limited to, viral IRES sequences, cellular IRES sequences, and artificial IRES sequences. Non-limiting examples of IRES sequences include Kaposi's sarcoma-associated herpesvirus (KSHV) IRES, hepatitis virus IRES, Pestivirus IRES, Cripavirus IRES, Rhopalosiphum padi virus IRES, fibroblast growth factor IRES, platelet-derived growth factor IRES, vascular endothelial growth factor IRES, insulin-like growth factor IRES, picornavirus IRES, encephalomyocarditis virus (EMCV) IRES, Pim-1 IRES, p53 IRES, Apaf-1 IRES, TDP2 IRES, L-myc IRES, and c-myc IRES.

In some embodiments, the 5′ flanking domain does not include a translation start site in any reading frame. In some embodiments, the 5′ flanking domain includes a translation start site or a part thereof (e.g., ending with an “A” or an “AT” or an “ATG”) as the last nucleotides of the 5′ adaptor sequence. In some embodiments, the 5′ flanking domain includes a methionine codon as the last three nucleotides of the 5′ adaptor sequence. In some embodiments, the 5′ flanking domain has a length of from about 15 nucleotides to about 35 nucleotides. In some embodiments, 5′ flanking domain has a length of about 30 nucleotides. In some embodiments, the 5′ flanking domain includes a nucleic acid sequence having at least 70% such as, for example, at least 75%, at least 80%, at least 85%, at least 90%, or at least 95% sequence identity to SEQ ID NO: 1. In some embodiments, the 5′ flanking domain includes a nucleic acid sequence having at least 96%, at least 97% at least 98%, or at least 99% sequence identity to SEQ ID NO: 1. In some embodiments, the 5′ flanking domain includes a nucleic acid sequence having 100% sequence identity to SEQ ID NO: 1. In some embodiments, the1′ flanking domain includes a nucleic acid sequence having 100% sequence identity to SEQ ID NO: 1, and further wherein one, two, three, four, or five nucleotides in the nucleic acid sequence is substituted by a different nucleotide.

As described above, in some embodiments of the disclosure, the 3′ flanking domain of the synthetic adaptor molecule includes a nucleic acid sequence predicted to have minimal secondary structure, such as a stem-loop structure. In some embodiments, the sequences of the 3′ flanking domain has a folding ΔG value of the minimum free energy (MFE) structure higher than a predefined threshold value. In some embodiments, the 3′ flanking domain does not include a sequence which encodes an RNA sequence capable of forming a stem-loop structure. In some embodiments, the 3′ flanking domain include a translation stop codon as the first three nucleotides of the 3′ adaptor sequence. Suitable stop codons include TAG, TAA, and TGA. Accordingly, in some embodiments, the 3′ flanking domain include a TAG stop codon as the first three nucleotides of the 3′ adaptor sequence. In some embodiments, the 3′ flanking domain include a TAA stop codon as the first three nucleotides of the 3′ adaptor sequence. In some embodiments, the 3′ flanking domain include a TAG stop codon as the first three nucleotides of the 3′ adaptor sequence. In some embodiments, the 3′ flanking domain includes a nucleic acid sequence having at least 70% such as, for example, at least 75%, at least 80%, at least 85%, at least 90%, or at least 95% sequence identity to SEQ ID NO: 2. In some embodiments, the 3′ flanking domain includes a nucleic acid sequence having at least 96%, at least 97% at least 98%, or at least 99% sequence identity to SEQ ID NO: 2. In some embodiments, the 3′ flanking domain includes a nucleic acid sequence having 100% sequence identity to SEQ ID NO: 2. In some embodiments, the 3′ flanking domain includes a nucleic acid sequence having 100% sequence identity to SEQ ID NO: 2, and further wherein one, two, three, four, or five nucleotides in the nucleic acid sequence is substituted by a different nucleotide.

In some embodiments, the synthetic adaptor molecule includes a nucleic acid sequence having at least 70% such as, for example, at least 75%, at least 80%, at least 85%, at least 90%, or at least 95% sequence identity to SEQ ID NO: 20. In some embodiments, the synthetic adaptor molecule includes a nucleic acid sequence having at least 96%, at least 97% at least 98%, or at least 99% sequence identity to SEQ ID NO: 20. In some embodiments, the synthetic adaptor molecule includes a nucleic acid sequence having 100% sequence identity to SEQ ID NO: 20. In some embodiments, the synthetic adaptor molecule includes a nucleic acid sequence having 100% sequence identity to SEQ ID NO: 20, and further wherein one, two, three, four, five, six, seven, eight, nine, or ten nucleotides in the nucleic acid sequence is substituted by a different nucleotide.

### Restriction Sites

In some embodiments, the restriction site in the synthetic adaptor molecule is cleavable by a restriction enzyme selected from Type I restriction enzymes, Type II restriction enzymes, Type III restriction enzymes, Type IV restriction enzymes, Type V restriction enzymes, and homing endonucleases. In some embodiments, the restriction site in the synthetic adaptor molecule is uniquely cleavable, e.g., a unique restriction site in the entire nucleic acid construct. In order to render the restriction site unique, silent mutations can optionally be engineered into restriction sites in the replicon-coding sequence of the nucleic acid construct.

In some embodiments, the restriction site is cleavable by a restriction enzyme selected from Type I restriction enzymes, which are complex, multi-subunit, combination restriction-and-modification enzymes that cut DNA at a site that differs, and is a random distance (at least 1000 bp) away, from their recognition site. Cleavage at these random sites follows a process of DNA translocation, which shows that these enzymes are also molecular motors. The recognition site is asymmetrical and is composed of two specific portions, one containing 3-4 nucleotides, and another containing 4-5 nucleotides, separated by a non-specific spacer of about 6-8 nucleotides. These enzymes are multifunctional and are capable of both restriction digestion and modification activities, depending upon the methylation status of the target DNA. The cofactors S-Adenosyl methionine (AdoMet), hydrolyzed adenosine triphosphate (ATP), and magnesium (Mg2+) ions, are required for their full activity.

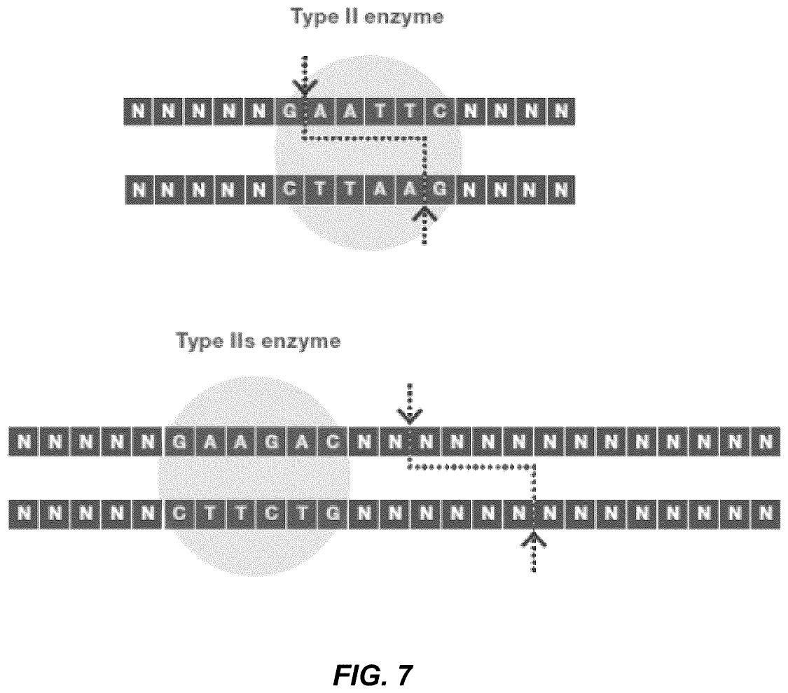

In some embodiments, the restriction site is cleavable by a restriction enzyme selected from Type II restriction enzymes, which recognize specific 4 to 8 nucleotide sequences that are typically palindromic and cleave at defined positions within the recognition sequences leaving sticky (5′ or 3′ overhangs) or blunt ends (see, e.g., FIG. 7). They produce discrete restriction fragments and distinct gel banding patterns, and they are often used in the laboratory for routine DNA analysis and gene cloning. Exemplary Type II enzymes include HhaI, HindIII, and NotI, that cleave DNA within their recognition sequences. Many Type II enzymes are available commercially. Most recognize DNA sequences that are symmetric, because they bind to DNA as homodimers, but a few, (e.g., BbvCI) recognize asymmetric DNA sequences, because they bind as heterodimers. Some Type II enzymes recognize continuous sequences (e.g., EcoRI) in which the two half-sites of the recognition sequence are adjacent, while others recognize discontinuous sequences (e.g., BglI) in which the half-sites are separated. Cleavage leaves a 3′-hydroxyl on one side of each cut and a 5′-phosphate on the other. Type II enzymes require magnesium for activity and the corresponding modification enzymes require S-adenosylmethionine. Type II enzymes tend to be small, with subunits in the 200-350 amino acid range. In some embodiments, the restriction site in the synthetic adaptor molecule is cleavable by SpeI or an isoschizomer thereof. Suitable isoschizomers of SpeI include, but are not limited to AhII, BcuI, and SpeI-HF.

In some embodiments, the restriction site in the synthetic adaptor molecule is cleavable by a Type IIS restriction enzyme. Type IIS restriction enzymes comprise a group of enzymes which cut DNA at a defined distance downstream or upstream of the recognition sequence. This is due to the enzyme architecture where the catalytic and recognition domains are separated by a polypeptide linker. There are no sequence requirements for the identity of bases in the cleavage site; therefore sequences beyond the recognition site can be any combination of nucleotides ((see, e.g., FIG. 7). Type IIS restriction enzymes include those like FokI and AlwI that cleave outside of their recognition sequence to one side. These enzymes are intermediate in size, 400-650 amino acids in length, and they recognize sequences that are continuous and asymmetric. They comprise two distinct domains, one for DNA binding, the other for DNA cleavage. They are believed to bind to DNA as monomers for the most part, but to cleave DNA cooperatively, through dimerization of the cleavage domains of adjacent enzyme molecules. For this reason, some Type IIS enzymes are much more active on DNA molecules that contain multiple recognition sites.

In some embodiments, the restriction site is cleavable by a restriction enzyme selected from Type III restriction enzymes (e.g., EcoP15), which are large combination restriction-and-modification enzymes. Type III restriction enzymes recognize two separate non-palindromic sequences that are inversely oriented. They cut DNA about 20-30 base pairs after the recognition site. These enzymes contain more than one subunit and require AdoMet and ATP cofactors for their roles in DNA methylation and restriction digestion, respectively. Type III restriction enzymes are components of prokaryotic DNA restriction-modification mechanisms that protect the organism against invading foreign DNA. Type III enzymes are hetero-oligomeric, multifunctional proteins composed of two subunits, Res (P08764) and Mod (P08763). The Mod subunit recognizes the DNA sequence specific for the system and is a modification methyltransferase; as such, it is functionally equivalent to the M and S subunits of type I restriction endonuclease. Res is required for restriction digestion, although it has no enzymatic activity on its own. Type III enzymes recognize short 5-6 bp-long asymmetric DNA sequences and cleave 25-27 bp downstream to leave short, single-stranded 5′ protrusions. They require the presence of two inversely oriented unmethylated recognition sites for restriction digestion to occur. These enzymes methylate only one strand of the DNA, at the N-6 position of adenosyl residues, so newly replicated DNA will have only one strand methylated, which is sufficient to protect against restriction digestion. Type III enzymes belong to the beta-subfamily of N6 adenine methyltransferases, containing the nine motifs that characterize this family, including motif I, the AdoMet binding pocket (FXGXG), and motif IV, the catalytic region (S/D/N (PP) Y/F). Additional information regarding Type I, II, III, and IV V DNA restriction systems be found in, for example, Leonen et al., Nucleic Acids Res (2014) 42(1):3-19), which is herein incorporated by reference.

In some embodiments, the restriction site is cleavable by a restriction enzyme selected from Type IV restriction enzymes, which recognize modified, optionally methylated DNA and are exemplified by the McrBC and Mrr systems of E. coli.

In some embodiments, the restriction site is cleavable by a restriction enzyme selected from Type V restriction enzymes, which utilize guide RNAs (gRNAs) to target specific non-palindromic sequences found on invading organisms. Type V restriction enzymes can cut DNA of variable length, provided that a suitable guide RNA is provided. Non-limiting examples of Type V restriction enzymes include the cas9-gRNA complex from CRISPRs.

In some embodiments, the restriction site is cleavable by a homing endonuclease (e.g., I-SceI). Homing endonucleases are double stranded DNases that have large, asymmetric recognition sites (12-40 base pairs) and coding sequences that are usually embedded in either introns or inteins. Generally, homing endonucleases cut DNA at a defined distance downstream or upstream of their large, asymmetric recognition sequences (12-40 base pairs). A large amount of biochemical and structural data has been reported for these enzymes over the past few decades, and can be found in, for example, Chevalier and Stoddard, Nucleic Acids Res (2001) 29(18): 3757-3774), which is herein incorporated by reference. Examples of homing endonucleases suitable for the compositions and methods of the disclosure include, but are not limited to, I-CeuI, I-SceI, PI-PspI, and PI-SceI.

In some embodiments, the nucleic acid constructs of the disclosure further include an additional restriction site incorporated immediately downstream of the sequence encoding the poly(A) tail of the alphavirus genome or replicon RNA. In instances in which the nucleic acid constructs are in circular form, the additional restriction site incorporated immediately downstream of the sequence encoding the poly(A) tail may facilitate the linearization of the circular nucleic acid constructs, thereby generating “clean” poly(A) template ends and/or generating nucleic acid products with the same end identity. In some embodiments, such restriction site may allow for generation of de-concatemerized rolling circle amplification (RCA) products or processing of polymerase chain reaction (PCR) products that leave the same end identity. One skilled in the art will appreciate that a “clean” poly(A) template end generally denotes a DNA sequence end with a homopolymeric sequence that templates for an RNA IVT product that terminates by run-off transcription, resulting in a RNA product containing a poly(A) sequence without 3′ non-A residues. In one aspect, some embodiments of the disclosure relate to nucleic acid constructs including a modified alphavirus genome or replicon RNA including a poly(A) tail, wherein an additional restriction site is engineered immediately downstream of the sequence encoding the poly(A) tail of the alphavirus genome or replicon RNA. In some embodiments, the additional restriction site is cleavable by a Type IIS restriction enzyme. Examples of Type IIS restriction enzymes suitable for the compositions and methods of the present disclosure include AcuI, AlwI, Alw26I, BaeI, BbiI, BbsI, BbsI-HF, BbvI, BccI, BceAI, BcgI, BciVI, BcoDI, BfuAI, BmrI, BpmI, BpuEI, BsaI, BsaI-HF, BsaI-HFv2, BsaXI, BseGI, BseRI, BsgI, BsmAI, BsmBI-v2, BsmFI, BsmI, BspCNI, BspMI, BspQI, BsrDI, BsrI, BtgZI, BtsCI, BtsI-v2, and BtsIMutI. Additional suitable Type IIS restriction enzymes include, but are not limited to, CspCI, EarI, EciI, Eco31I, Esp3I, FauI, FokI, HgaI, HphI, HpyAV, LpuI, MboII, MlyI, Mmel, MnlI, NmeAIII, PaqCI, PleI, SapI, and SfaNI. In some embodiments, the additional restriction site is cleavable by SapI, BpiI, BmsI, Mva1269I or an isoschizomer of any thereof. In some embodiments, the additional restriction site is cleavable by SapI or an isoschizomer thereof. In some embodiments, the isoschizomer of SapI is LguI, PciSI, or BspQI.

The demonstration that the modified alphavirus genomes or replicon RNAs (e.g., srRNAs) as disclosed herein, for example, those including a restriction site incorporated downstream of the sequence encoding the poly(A) tail resulting modified alphavirus genomes or replicon RNAs (e.g., srRNAs) without non-adenylate residues at the 3′ terminus, demonstrate surprisingly enhanced biologic activity since replicons in the state-of-the-art most commonly contain non-adenylate residues on the 3′ terminus. In some embodiments, the level of replication, expression, and/or translation enhancement activity of the modified genomes or replicon RNAs (e.g., srRNAs) as disclosed herein is of at least 1.1, 1.2, 1.3, 1.4, 1.5, 1.6, 1.7, 1.8, 1.9, 2 (2-fold), 3, 4, 5, 6, 7, 8, or more times, relative to the replication, expression, or translation level detected from a corresponding unmodified replicon (e.g., srRNA), e.g. replicon (e.g., srRNA) with non-adenylate residues on the 3′ terminus. In some embodiments, the level of replication, expression, and/or translation enhancement activity of the modified genomes or replicon RNAs (e.g., srRNAs) as disclosed herein is increased by at least 30%, at least 40%, at least 50%, at least 60%, at least 70%, at least 80%, at least 90%, or at least 100%, relative to the replication, expression, or translation level detected from a corresponding unmodified replicon (e.g., srRNA), e.g. replicon (e.g., srRNA) with non-adenylate residues on the 3′ terminus. The level of enhancement activity can be measured by any convenient methods and techniques known in the art including, but are not limited to, transcript level, amount of protein, protein activity, etc. In some embodiments, the level of enhancement activity can be evidenced by a higher percentage of the cells containing double-stranded RNA at a given mass (dose) of RNA transformed into cells in tissue culture. In some embodiments, the level of enhancement activity can be evidenced by a higher percentage of the cells expressing a protein at a given mass (dose) of RNA transformed into cells in tissue culture.

Without being limited by any particular theory, an enhanced replication, expression, or translation level can be due to the absence of non-A nucleotides at the 3′ end of the recombinant RNA molecule, which do not canonically appear in normal alphavirus biology. The modified alphavirus design described herein is in stark contrast to existing alphavirus vectors where SP6 or T7 RNA polymerase is often used to transcribe the RNA product, which terminates while transcribing a sequence (containing non-As) downstream of the poly(A), in a feature known as a “terminator,” or where a restriction enzyme is used to linearize the template encoding the RNA product which terminates by run-off transcription but results in non-adenylate residues to be incorporated at the 3′ terminus of the RNA.

As described in greater detail below, the incorporation of a Type IIS restriction enzyme downstream of the poly(A) tail that is subsequently cleaved to generate a linear DNA template causes termination of transcription by run-off transcription without the presence of an RNA polymerase terminator sequence. In the experiments described below, the Type IIS restriction endonuclease site is a SapI site, which cleaves upstream of the SapI recognition sequence, leaving only a poly(A) template on the 3′ end of the linearized DNA (i.e., no non-A nucleotides would be in the DNA template or the transcribed RNA product). This approach has not been described for replicons and the presence of exclusively adenylate residues in the poly(A) tail has not been described to confer any enhancement of biologic activity to replicons, where the most common methods are using a transcription terminator or run-off transcription, which both typically leave non-adenylate nucleotides at the end of the transcription product, or enzymatic poly(A) tailing of an in vitro transcribed product which still contain non-adenylate residues after the 3′ UTR.

As discussed above, it has been previous reported that for alphavirus genomes to replicate, 11 residues in the poly(A) tail following the 3′ UTR are necessary to efficiently initiate minus-strand synthesis, and therefore replication to occur. In addition, internal non-A residues in the poly(A) are most often deleterious to replication, which suggests that enzymatic poly(A) tailing would not benefit replicon RNA that did not exclusively contain 3′ adenylate residues following the 3′ UTR. It has been previously reported that there is no enhancement of minus-strand synthesis on RNA templates with greater than 25 adenylate residues in the poly(A) tail, for example with 34 adenylate residues in the poly(A) tail. Additional information in this regard can be found in, for example, Hardy & Rice, J. Virol. Pp. 4630-4639, April 2005.

In some embodiments of the disclosure, the poly(A) tail of the alphavirus genome or replicon RNA (e.g., srRNA) is lengthened by increasing the length of the poly(A) on the DNA template to enhance replication, expression, or translation level which is unexpected based on reported alphavirus biology or alphavirus replicons. In particular, experimental data presented herein has demonstrated a surprising change (e.g., increase) in the level of biologic activity in the form of RNA replication and protein expression by increasing the length of the poly(A) tail. In some embodiments, the lengthened sequence encoding the poly(A) tail has a length ranging from about 30 to about 120 adenylate residues, such as, for example, from about 30 to about 60, about 40 to about 70, about 50 to about 80, about 60 to about 90, about 70 to about 100, about 40 to about 80, about 50 to about 70, about 60 to about 90, or about 40 to about 90 adenylate residues. In some embodiments, the lengthened poly(A) tail is longer than about 34 residues. In some embodiments, the lengthened poly(A) tail has a length of about 30, about 40, about 50, about 60, about 70, about 80, about 90, and about 100 adenylate residues. In some embodiments, the lengthened poly(A) tail has a length of 30 adenylate residues. In some embodiments, the lengthened poly(A) tail has a length of 49 adenylate residues. In some embodiments, the lengthened poly(A) tail has a length of 91 adenylate residues. In some embodiments, the lengthened poly(A) tail has a length of 90 adenylate residues. In some embodiments, the lengthened poly(A) tail has a length of 64 adenylate residues.

The level of enhanced activity can be measured by any suitable methods and techniques known in the art including, but are not limited to, those methods and techniques that measure transcript level, amount of protein, and/or protein activity, etc.

In some embodiments, the nucleic acid construct includes a modified replicon RNA (e.g., srRNA) comprising a modified genome or replicon RNA (e.g., srRNA) of a virus belonging to the Alphavirus genus of the Togaviridae family. Virulent and avirulent alphavirus strains are both suitable. In some embodiments, the modified genome or replicon RNA is of an alphavirus belonging to the VEEV/EEEV group, or the SFV group, or the SINV group. In some embodiments, the alphavirus is selected from the group consisting of Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus. In some embodiments, the alphavirus is Venezuelan equine encephalitis virus (VEEV). In some embodiments, the alphavirus is Chikungunya virus (CHIKV). In some embodiments, the alphavirus is Sindbis virus (SINV). In some embodiments, the alphavirus is Eastern Equine Encephalitis virus (EEEV).

Non-limiting examples of CHIKV strains suitable for the compositions and methods of the disclosure include CHIKV S27, CHIKV LR2006-OPY-1, CHIKV YO123223, CHIKV DRDE, CHIKV 37997, CHIKV 99653, CHIKV Ag41855, and Nagpur (India) 653496 strain. Additional examples of CHIKV strains suitable for the compositions and methods of the disclosure include but are not limited to those described in Afreen et al. Microbiol. Immunol. 2014, 58:688-696, Lanciotti and Lambert ASTMH 2016, 94(4):800-803 and Langsjoen et al. mBio. 2018, 9(2): e02449-17. In some embodiments, the modified CHIKV genome or replicon RNA (e.g., srRNA) is derived from CHIKV strain S27. In some embodiments, the modified CHIKV genome or replicon RNA is derived from CHIKV strain DRDE. In some embodiments, the modified CHIKV genome or replicon RNA (e.g., srRNA) is derived from CHIKV strain DRDE-06. In some embodiments, the modified CHIKV genome or replicon RNA (e.g., srRNA) is derived from CHIKV strain DRDE-07.

Non-limiting examples of SINV strains suitable for the compositions and methods of the disclosure include SINV strain AR339, AR86, and Girdwood. Additional examples of SINV strains suitable for the compositions and methods of the disclosure include but are not limited to those described in Sammels et al. J. Gen. Virol. 1999, 80(3): 739-748, Lundström and Pfeffer Vector Borne Zoonotic Dis. 2010, 10(9):889-907, Sigei et al. Arch. of Virol. 2018, 163:2465-2469 and Ling et al. J. Virol. 2019, 93:e00620-19. In some embodiments, the modified SINV genome or replicon RNA (e.g., srRNA) is derived from SINV strain Girdwood. In some embodiments, the modified SINV genome or replicon RNA (e.g., srRNA) is a chimera of SINV strain Girdwood and SINV strain AR86.

Non-limiting examples of VEEV strains suitable for the compositions and methods of the disclosure include 204381, 306425, 3880, 3908, 6119, 66637, 68U201, 69Z1, 83U434, 93-42124, 96-32863, AB66640, An9004, C-84, CPA-201, FSL0201, INH-6803, INH-9813, Pan36080, P676, SH3, TC-83, TRD, V178, V198, V209A, V3526, and ZPC738.

Non-limiting examples of EEEV strains suitable for the compositions and methods of the disclosure include 300851, 436087, 783372, 792138, AR36, AR38, AR59, BG60, BR56, BR60, BR65, BR67, BR75, BR76, BR77, BR78, BR83, BR85, C-49, CO92, CT90, EC74, FL02a-b, FL82, FL91, FL93-1637, FL93-939, FL93-969, FL96, GA01, GA91, GA97, GML, GML903836, GU68, LA02, LA47, LA50, MA06, MA38, MA77, MD85, MD90A, MP-9, MS83, MX97, NJ03a-b, NJ60, NY03a-d, NY04a-k, NY05a-f, NY69, NY71a-c, NY73, NY74a-h, NY75, PA62, PA84, PA86, PE-0.0155-96, PE-16.0050-98, PE-18.0140-99, PE-18.0172-99, PE-3.0815-96, PE6, PE70, PE75, TN08, TR59, TVP8512, TX03, TX91, TX95, VA03, VA33, VA33, VE76, VE80, and W180. In some embodiments, the modified EEEV genome or replicon RNA (e.g., srRNA) is derived from EEEV strain FL93-939.

Non-limiting examples of WEEV strains suitable for the compositions and methods of the disclosure include WEEV California, McMillan, IMP181, Imperial, Imperial181, IMPR441, 71V-1658, AG80-646, BFS932, COA592, EP-6, E1416, BFS1703, BFS2005, BSF3060, BSF09997, CHLV53, KERN5547, 85452NM, Montana-64, S8-122, and TBT-235. Additional examples of WEEV strains suitable for the compositions and methods of the disclosure include 5614, 93A27, 93A30, 93A38, 93A79, B628(Cl 15), CBA87, CNTR34, CO921356, Fleming, Lake43, PV012357A, PV02808A, PV72102, R02PV001807A, R02PV002957B, R02PV003422B, R05PV003422B, R0PV003814A and R0PV00384A. Additional suitable WEEV strains include, but are not limited to those described in Bergren N A et al., J. Virol. 88(16): 9260-9267, August 2014, and in the Virus Pathogen Resource website (ViPR; which is publicly available at www.viprbrc.org/brc/vipr_genome_search.spg?method=SubmitForm&blockId=868&decorator=toga). In some embodiments, the modified WEEV genome or srRNA is derived from WEEV strain Imperial.

In some embodiments, the nucleic acid constructs of the disclosure further include one or more expression cassettes. In principle, the nucleic acid constructs disclosed herein can generally include any number of expression cassettes. In some embodiments, the nucleic acid constructs disclosed herein can include at least two, at least three, at least four, at least five, or at least six expression cassettes. The skilled artisan will understand that the term “expression cassette” refers to a construct of genetic material that contains coding sequences and enough regulatory information to direct proper transcription and/or translation of the coding sequences in a cell, in vivo and/or ex vivo. The expression cassette may be inserted into a vector for targeting to a desired host cell and/or into a subject. Accordingly, in some embodiments, the term expression cassette may be used interchangeably with the term “expression construct.” In some embodiments, the term “expression cassette” refers to a nucleic acid construct that includes a gene encoding a protein or functional RNA operably linked to regulatory elements such as, for example, a promoter and/or a termination signal, and optionally, any or a combination of other nucleic acid sequences that affect the transcription or translation of the gene.

In some embodiments, at least one of the expression cassettes includes a promoter operably linked to a heterologous nucleic acid sequence. Accordingly, the nucleic acid constructs as provided herein can find use, for example, as an expression vector that, when including a regulatory element (e.g., a promoter) operably linked to a heterologous nucleic acid sequence, can affect expression of the heterologous nucleic acid sequence. In some embodiments, at least one of the expression cassettes includes a subgenomic (sg) promoter operably linked to a heterologous nucleic acid sequence. In some embodiments, the sg promoter is a 26S subgenomic promoter. In some embodiments, the nucleic acid molecules of the disclosure further include one or more untranslated regions (UTRs). In some embodiments, at least one of the UTRs is a heterologous UTR. In some embodiments, at least one of the heterologous UTRs includes a sequence having at least 80%, at least 85%, at least 90%, at least 95%, at least 96%, at least 97%, at least 98%, at least 99%, or 100% sequence identity to the nucleic acid sequence of SEQ ID NO: 16. In some embodiments, at least one of the heterologous UTRs includes a sequence having at least 80%, at least 85%, at least 90%, at least 95%, at least 96%, at least 97%, at least 98%, at least 99%, or 100% sequence identity to the nucleic acid sequence of SEQ ID NO: 17.

In some embodiments, at least one of expression cassettes includes a coding sequence for a gene of interest (GOI). In some embodiments, the GOI coding sequence includes a stop codon positioned upstream of the 3′ flanking domain of the synthetic adaptor molecule. In some embodiments, the coding sequence of the GOI is optimized for a desired property. For example, in some embodiments, the coding sequence of the GOI is optimized for expression at a level higher than the expression level of a reference coding sequence. With respect to sequence-optimization of nucleotide sequences, degeneracy of the genetic code provides the possibility to substitute at least one base of the protein encoding sequence of a gene with a different base without causing the amino acid sequence of the polypeptide produced from the gene to be changed. Hence, the nucleic acid constructs of the present disclosure may also have any base sequence that has been changed from any polynucleotide sequence disclosed herein by substitution in accordance with degeneracy of the genetic code. References describing codon usage are readily publicly available. In some embodiments, polynucleotide sequence variants can be produced for a variety of reasons, e.g., to optimize expression for a particular host (e.g., changing codon usage in the alphavirus mRNA to those preferred by other organisms such as human, non-human primates, hamster, mice, or monkey). Accordingly, in some embodiments, the coding sequence of the GOI is optimized for expression in a target host cell through the use of codons optimized for expression. The techniques for the construction of synthetic nucleic acid sequences encoding GOI using preferred codons optimal for host cell expression may be determined by computational methods analyzing the commonality of codon usage for encoding native proteins of the host cell genome and their relative abundance by techniques well known in the art. The codon usage database (http://www.kazusa.or.jp/codon) may be used for generation of codon optimized sequences in mammalian cell environments. Furthermore, a variety of software tools are available to convert sequences from one organism to the optimal codon usage for a different host organism such as the JCat Codon Optimization Tool (www.jcat.de), Integrated DNA Technologies (IDT) Codon Optimization Tool (https://www.idtdna.com/CodonOpt) or the Optimizer online codon optimization tool (http://genomes.urv.es/OPTIMIZER). Such synthetic sequences may be constructed by techniques known in the art for the construction of synthetic nucleic acid molecules and may be obtained from a variety of commercial vendors. Accordingly, in some embodiments, the coding sequence of the GOI is optimized for expression at a level higher than the expression level of a reference coding sequence, such as, for example, a coding sequence that has not been codon-optimized. In some embodiments, the codon-optimized sequence of the GOI results in an increased expression level by at least 30%, at least 40%, at least 50%, at least 60%, at least 70%, at least 80%, at least 90%, at least 95%, or at least 100% compared to a reference coding sequence that has not been codon-optimized. In some embodiments, the codon-optimized sequence of the GOI results in an increased expression level by at least 2-fold, at least 3-fold, at least 4-fold, or at least 5-fold compared to a reference coding sequence that has not been codon-optimized.

The polypeptide encoded by a GOI can generally be any polypeptide, and can be, for example a therapeutic polypeptide, a prophylactic polypeptide, a diagnostic polypeptide, a nutraceutical polypeptide, an industrial enzyme, and a reporter polypeptide. In some embodiments, the GOI encodes a polypeptide that can be an antibody, an antigen, an immune modulator, an enzyme, a signaling protein, or a cytokine. In some embodiments, the GOI can encode microbial proteins, viral proteins, bacterial proteins, fungal proteins, mammalian proteins, and combinations of any thereof. In some embodiments, the GOI encodes a hemagglutinin precursor (HA) of the influenza A virus H5N1. Non-limiting examples of GOI include interleukins and interacting proteins, including: G-CSF, GM-CSF, IL-1, IL-10, IL-10-like, IL-11, IL-12, IL-13, IL-14, IL-15, IL-16, IL-17, IL-18, IL-18BP, IL-1-like, IL-IRA, IL-la, IL-1B, IL-2, IL-20, IL-3, IL-4, IL-5, IL-6, IL-6-like, IL-7, IL-9, IL-21, IL-22, IL-33, IL-37, IL-38, LIF, and OSM. Additional suitable GOIs include, but are not limited to, interferons (e.g., IFN-α, IFN-β, IFN-γ), TNFs (e.g., CD154, LT-β, TNF-α, TNF-β, 4-1BBL, APRIL, CD70, CD153, CD178, GITRL, LIGHT, OX40L, TALL-1, TRAIL, TWEAK, and TRANCE), TGF-β (e.g., TGF-β1, TGF-β2, and TGF-β3), hematopoietins (e.g., Epo, Tpo, Flt-3L, SCF, M-CSF, MSP), chemokines and their receptors (e.g., XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, and CX3CL1), immunosuppressive gene products and related transcription factors (e.g., PECAM1, FCGR3A, FOS, NFKB1, JUN, HIF1A, PD-L1, mTOR, STAT5B, and STAT4). Additional GOIs suitable for the compositions and methods of the disclosure include, but are not limited to, immunostimulatory gene products (e.g., CD27/CD70, CD40, CD40L, B7.1, BTLA, MAVS, OX40, OX40L, RIG-I, and STING), drug resistant mutants/variants of genes, such as ABCB1, ABCC1, ABCG2, AKT1, ALK, BAFF, BCR-ABL, BRAF, CCND1, cMET, EGFR, ERBB2, ERBB3, ERK2, ESR1, GRB2, KRAS, MDR1, MRP1, NTRK1, PDC4, P-gp, PI3K, PTEN, RET, ROS1, RSK1, RSK2, SHIP, and STK11. Also suitable for the compositions and methods of the disclosure includes sequence encoding viral proteins, in particular spike proteins, fiber proteins, structural proteins, and attachment proteins.

In some embodiments, the GOI can encode an antibody or antibody variant (e.g. single chain Fv, bi-specifics, camelids, Fab, and HCAb). In some embodiments, the antibody targets surface molecules associated or upregulated with cancers, or surface molecules associated with infectious disease. In some embodiments, the antibody targets surface molecules having immunostimulatory function, or having immunosuppressive function.

In some embodiments, the GOI can encode an enzyme whose deficiency or mutation is associated with diseases or health conditions, such as, for example, agalsidase beta, agalsidase alfa, imiglucerase, taliglucerase alfa, velaglucerase alfa, alglucerase, sebelipase alpha, laronidase, idursulfase, elosulfase alpha, galsulfase, alglucosidase alpha, and CTFR.

In some embodiments, the GOI can encode a polypeptide selected from antigen molecules, biotherapeutic molecules, or combinations of any thereof. In some embodiments, the GOI can encode a polypeptide selected from tumor-associated antigens, tumor-specific antigens, neoantigens, and combinations of any thereof. In some embodiments, the GOI can encode a polypeptide selected from estrogen receptors, intracellular signal transducer enzymes, and human epidermal growth receptors. In some embodiments, the GOI can encode a biotherapeutic polypeptide selected from immunomodulators, modulators of angiogenesis, modulators of extracellular matrix, modulators of metabolism, neurological modulators, and combinations of any thereof. In some embodiments, the GOI can encode a cytokine selected from chemokines, interferons, interleukins, lymphokines, and tumor necrosis factors. In some embodiments, the GOI can encode an interleukins selected from IL-la, IL-1B, IL-2, IL-3, IL-4, IL-5, IL-6, IL-7, IL-8, IL-9, IL-10, IL-11, IL-12, IL-15, IL-15, IL-17, IL-23, IL-27, IL-35, IFNγ and subunits of any thereof. In some embodiments, the GOI can encode a biotherapeutic polypeptide is selected from IL-12A, IL-12B, IL-IRA, and combinations of any thereof.

In some embodiments, the coding sequence of the GOI does not contain restriction enzyme site(s) that are used to linearize the nucleic acid construct encoding the modified alphavirus genome or replicon RNA (e.g., srRNA). In some embodiments, the nucleic acid construct of the disclosure may be incorporated within a vector. In some embodiments, the vector of the disclosure may be single-stranded vector, e.g., ssDNA vector or ssRNA vector. In some embodiments, the vector of the disclosure can be double-stranded vector, e.g., dsDNA vector or dsRNA vector. In some embodiments, the vector of the disclosure can be a plasmid. As described in greater detail below, the vector of the disclosure can be produced using recombinant DNA technology, e.g., polymerase chain reaction (PCR) amplification, rolling circle amplification (RCA), molecular cloning, etc., or chemical synthesis. Accordingly, in some embodiments, the vector of the disclosure can be a fully synthetic vector, e.g., fully synthetic ssDNA vector. In some embodiments, the vector of the disclosure can be a fully synthetic dsDNA vector. In some embodiments, the vector of the disclosure can be a product of a PCR reaction. In some embodiments, the vector of the disclosure can be a product of a RCA reaction. In some embodiments, a vector can be a gene delivery vector. In some embodiments, a vector can be used as a gene delivery vehicle to transfer a gene into a cell.

In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to a nucleic acid sequence selected from the group consisting of SEQ ID NOs: 3-27. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 3. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 4. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 5. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 6. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 22. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 23. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 24. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 25. In some embodiments, the nucleic acid constructs of the disclosure include a nucleic acid sequence encoding a modified alphavirus having at least 80%, at least 85%, at least 90%, at least 95%, at least 96%, at least 97%, at least 98%, at least 99%, or 100% sequence identity to the nucleic acid sequence of SEQ ID NO: 27.

Nucleic acid sequences having a high degree of sequence identity (e.g., at least 80%, at least 85%, at least 90%, at least 95%, at least 96%, at least 97%, at least 98%, at least 99%, or 100%) to a sequence of a modified alphavirus of interest can be identified and/or isolated by using the sequences identified herein (e.g., SEQ ID NOS: 3-27) or any others as they are known in the art, by genome sequence analysis, hybridization, and/or PCR with degenerate primers or gene-specific primers from sequences identified in the respective alphavirus genome.

The molecular techniques and methods by which these new nucleic acid constructs were assembled and characterized are described more fully in the Examples herein of the present application. In the Examples section, the Chikungunya virus (CHIKV), Sindbis virus (SINV), Eastern Equine Encephalitis virus (EEEV), and Venezuelan equine encephalitis (VEE) virus have been used to illustrate the compositions and methods disclosed herein.

In some embodiments, the nucleic acid molecules are recombinant nucleic acid molecules. As used herein, the term recombinant means any molecule (e.g. DNA, RNA, polypeptide), that is, or results, however indirect, from human manipulation. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature; 3) has been engineered using molecular cloning techniques such that it lacks one or more nucleotides with respect to the naturally occurring nucleotide sequence; and/or 4) has been manipulated using molecular cloning techniques such that it has one or more sequence changes or rearrangements with respect to the naturally occurring nucleotide sequence.

In some embodiments, the nucleic acid molecules disclosed herein are produced using recombinant DNA technology (e.g., polymerase chain reaction (PCR) amplification, cloning, etc.) or chemical synthesis. Nucleic acid molecules as disclosed herein include natural nucleic acid molecules and homologs thereof, including, but not limited to, natural allelic variants and modified nucleic acid molecules in which one or more nucleotide residues have been inserted, deleted, and/or substituted, in such a manner that such modifications provide the desired property in effecting a biological activity as described herein.

A nucleic acid molecule, including a variant of a naturally-occurring nucleic acid sequence, can be produced using a number of methods known to those skilled in the art (see, for example, Sambrook et al., In: Molecular Cloning, A Laboratory Manual, 2nd Edition, Cold Spring Harbor Press, Cold Spring Harbor, N.Y. (1989)). The sequence of a nucleic acid molecule can be modified with respect to a naturally-occurring sequence from which it is derived using a variety of techniques including, but not limited to, classic mutagenesis techniques and recombinant DNA techniques, such as but not limited to site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, recombinational cloning, and chemical synthesis, including chemical synthesis of oligonucleotide mixtures and ligation of mixture groups to “build” a mixture of nucleic acid molecules, and combinations thereof. Nucleic acid molecule homologs can be selected from a mixture of modified nucleic acid molecules by screening for the function of the protein or the replicon (e.g., srRNA) encoded by the nucleic acid molecule and/or by hybridization with a wild-type gene or fragment thereof, or by PCR using primers having homology to a target or wild-type nucleic acid molecule or sequence.

### B. Recombinant Cells and Cell Cultures

As described in greater detail below, one aspect of the present disclosure relates to recombinant cells that have been engineered to include a nucleic acid construct as described herein and/or include (e.g., express) a nucleic acid construct as described herein. In some embodiments, a nucleic acid construct (e.g., vector or srRNA) of the present disclosure can be introduced into a host cell to produce a recombinant cell containing the nucleic acid construct and/or srRNA construct. For example, the nucleic acid constructs of the present disclosure can be introduced into a host cell such as, for example, a Chinese hamster ovary (CHO) cell, to produce a recombinant cell containing the nucleic acid molecule. Accordingly, prokaryotic or eukaryotic cells that contain a nucleic acid construct as described herein are also features of the disclosure. In a related aspect, some embodiments disclosed herein relate to methods of transforming a cell which includes introducing into a host cell, such as an animal cell, a nucleic acid construct as provided herein, and then selecting or screening for a transformed cell. Introduction of the nucleic acid constructs (e.g., DNA or RNA, including mRNA) or vectors of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like. For example, methods for introduction of heterologous nucleic acid molecules into mammalian cells are known in the art and include dextran-mediated transfection, calcium phosphate precipitation, polybrene-mediated transfection, protoplast fusion, electroporation, encapsulation of the nucleic acid molecule(s) in liposomes, lipid nanoparticle technology, biolistic injection and direct microinjection of the DNA into nuclei.

In one aspect, some embodiments of the disclosure relate to recombinant cells, for example, recombinant eukaryotic cells, e.g., animal cells that include a nucleic acid construct described herein. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome editing techniques such as using guide RNA directed CRISPR/Cas9 or TALEN genome editing. In some embodiments, the nucleic acid construct present in the recombinant host cell as a mini-circle expression vector for a stable or transient expression.

Host cells can be either untransformed cells or cells that have already been transfected with at least one nucleic acid molecule. Accordingly, in some embodiments, host cells can be genetically engineered (e.g., transduced or transformed or transfected) with at least one nucleic acid molecule.

Suitable host cells for cloning or expression of the protein of interest as described herein include prokaryotic or eukaryotic cells described herein. Accordingly, in some embodiments, the recombinant cell of the disclosure is a prokaryotic cell, such as the bacterium E. coli, or a eukaryotic cell, such as an insect cell (e.g., a mosquito cell or a Sf21 cell), or mammalian cells (e.g., COS cells, NIH 3T3 cells, or HeLa cells). In some embodiments, the recombinant cell is a prokaryotic cell. In some embodiments, the prokaryotic cell is an E. coli cell. For example, a protein of interest may be produced in bacteria, in particular when glycosylation and Fc effector function are not needed. After expression, the protein of interest may be isolated from the bacterial cell paste in a soluble fraction and can be further purified.

In some embodiments, the cell is in vivo, for example, a recombinant cell in a living body, e.g., cell of a transgenic subject. In some embodiments, the subject is a vertebrate animal or an invertebrate animal. In some embodiments, the subject is an insect. In some embodiments, the subject is a mammalian subject. In some embodiments, the recombinant cell is a eukaryotic cell. In some embodiments, the cell is in vivo. In some embodiments, the cell is ex vivo, e.g., has been extracted, as an individual cell or as part of an organ or tissue, from a living body or organism for a treatment or procedure, and then returned to the living body or organism. In some embodiments, the cell is in vitro, e.g., is obtained from a repository.

In some embodiments of the disclosure, the recombinant cell of the disclosure is a eukaryotic cell. In some embodiments, the recombinant cell is an animal cell. In some embodiments, the animal cell is a vertebrate animal cell or an invertebrate animal cell. In some embodiments, the recombinant animal cell is a mammalian cell. Suitable host cells for the expression of glycosylated protein can be derived from multicellular organisms (invertebrates and vertebrates). Examples of invertebrate cells include insect cells.

Vertebrate cells can also be used as hosts. In this regard, mammalian cell lines that are adapted to grow in suspension can be useful. In some embodiments, the recombinant cell is an animal cell. In some embodiments, the animal cell is a vertebrate animal cell or an invertebrate animal cell. In some embodiments, the recombinant cell is a mammalian cell. In some embodiments, the animal cell is a human cell. In some embodiments, the animal cell is a non-human animal cell. In some embodiments, the cell is a non-human primate cell. Additional examples of useful mammalian host cell lines are monkey kidney CV1 line transformed by SV40 (COS-7), human embryonic kidney line (e.g., 293 or 293 cells), baby hamster kidney cells (BHK), mouse sertoli cells (e.g., TM4 cells), monkey kidney cells (CV1), African green monkey kidney cells (VERO-76), human cervical carcinoma cells (HELA), canine kidney cells (MDCK; buffalo rat liver cells (BRL 3A), human lung cells (W138), human liver cells (Hep G2), mouse mammary tumor (MMT 060562), TRI cells, MRC 5 cells; and FS4 cells. Other useful mammalian host cell lines include Chinese hamster ovary (CHO) cells, including DHFR-CHO cells, and myeloma cell lines such as YO, NSO and Sp2/0.

In some embodiments, the recombinant cell is selected from the group consisting of African green monkey kidney cell (Vero cell), baby hamster kidney (BHK) cell, Chinese hamster ovary cell (CHO cell), human A549 cell, human cervix cell, human CHME5 cell, human epidermoid larynx cell, human fibroblast cell, human HEK-293 cell, human HeLa cell, human HepG2 cell, human HUH-7 cell, human MRC-5 cell, human muscle cell, mouse 3T3 cell, mouse connective tissue cell, mouse muscle cell, and rabbit kidney cell.

In some embodiments of the disclosure, the recombinant cell is an insect cell, e.g., cell of an insect cell line. In some embodiments, the insect cell is a Sf21 cell. Additional suitable insect cell lines include, but are not limited to, cell lines established from insect orders Diptera, Lepidoptera and Hemiptera, and can be derived from different tissue sources. In some embodiments, the recombinant cell of the disclosure is a cell of a lepidopteran insect cell line. In the past few decades, the availability of lepidopteran insect cell lines has increased at about 50 lines per decade. More information regarding available lepidopteran insect cell lines can be found in, e.g., Lynn D. E., Available lepidopteran insect cell lines. Methods Mol. Biol. 2007; 388:117-38, which is herein incorporated by reference. In some embodiments, the recombinant cell is a mosquito cell, e.g., a cell of mosquito species within Anopheles (An.), Culex (Cx.) and Aedes (Stegomyia) (Ae.) genera. Exemplary mosquito cell lines suitable for the compositions and methods described herein include cell lines from the following mosquito species: Aedes aegypti, Aedes albopictus, Aedes pseudoscutellaris, Aedes triseriatus, Aedes vexans, Anopheles gambiae, Anopheles stephensi, Anopheles albimanus, Culex quinquefasciatus, Culex theileri, Culex tritaeniorhynchus, Culex bitaeniorhynchus, and Toxorhynchites amboinensis. Suitable mosquito cell lines include, but are not limited to, CCL-125, Aag-2, RML-12, C6/26, C6/36, C7-10, AP-61, A.t. GRIP-1, A.t. GRIP-2, UM-AVE1, Mos.55, Sua1B, 4a-3B, Mos.43, MSQ43, and LSB-AA695BB. In some embodiments, the mosquito cell is a cell of a C6/26 cell line.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### B. Transgenic Animals

Also provided, in another aspect, are transgenic animals including a nucleic acid construct as described herein. In some embodiments, the transgenic animal is a vertebrate animal or an invertebrate animal. In some embodiments, the transgenic animal is a mammalian. In some embodiments, the transgenic mammalian is a non-human mammalian. In some embodiments, the transgenic animal produces a recombinant RNA molecule as described herein. In some embodiments, the transgenic animal produces a protein of interest as described herein.

The transgenic non-human host animals of the disclosure are prepared using standard methods known in the art for introducing exogenous nucleic acid into the genome of a non-human animal. In some embodiments, the non-human animals of the disclosure are non-human primates. Other animal species suitable for the compositions and methods of the disclosure include animals that are (i) suitable for transgenesis and (ii) capable of rearranging immunoglobulin gene segments to produce an antibody response. Examples of such species include but are not limited to mice, rats, hamsters, rabbits, chickens, goats, pigs, sheep and cows. Approaches and methods for preparing transgenic non-human animals are known in the art. Exemplary methods include pronuclear microinjection, DNA microinjection, lentiviral vector mediated DNA transfer into early embryos and sperm-mediated transgenesis, adenovirus mediated introduction of DNA into animal sperm (e.g., in pig), retroviral vectors (e.g., avian species), somatic cell nuclear transfer (e.g., in goats). The state of the art in the preparation of transgenic domestic farm animals is reviewed in Niemann, H. et al. (2005) Rev. Sci. Tech. 24:285-298.

In some embodiments, the animal is a vertebrate animal or an invertebrate animal. In some embodiments, the animal is a mammalian subject. In some embodiments, the mammalian animal is a non-human animal. In some embodiments, the mammalian animal is a non-human primate. In some embodiments, the transgenic animals of the disclosure can be made using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the transgenic animals of the disclosure can be made using transgenic microinjection technology and do not require the use of homologous recombination technology and thus are considered to be easier to prepare and select than approaches using homologous recombination.

In another aspect, provided herein are methods for producing a recombinant RNA molecule, the methods include (i) rearing a transgenic animal as described herein, or (ii) culturing a recombinant cell as described herein under conditions such that the recombinant RNA molecule is produced by the transgenic animal or in the recombinant cell.

In some embodiments, the transgenic animal or the recombinant cell including a nucleic acid construct as described herein and wherein the sequence encoding the recombinant RNA molecule is optionally digested by a restriction enzyme capable of cleaving the restriction site engineered after the end of the sequence encoding the poly(A) tail to generates a template that encodes for an RNA that only has adenylate residues in the poly(A) tail and 3′ terminus. Accordingly, recombinant RNA molecules produced according to a method described herein are also provided by the present disclosure.

In some embodiments, the transgenic animal or the recombinant cell including a nucleic acid construct as described herein and wherein the sequence encoding the recombinant RNA molecule contains a lengthened poly(A) tail. Accordingly, recombinant RNA molecules produced according to a method described herein are also provided by the present disclosure.

In another aspect, provided herein are methods for producing a polypeptide of interest, wherein the methods include (i) rearing a transgenic animal comprising a nucleic acid construct as described herein, or (ii) culturing a recombinant cell including a nucleic acid construct as described herein under conditions wherein the polypeptide encoded by the GOI is produced by the transgenic animal or in the recombinant cell. In another aspect, provided herein are methods for producing a polypeptide of interest, the methods include administering to the subject a nucleic acid construct described herein. Non-limiting exemplary embodiments of the methods of the disclosure can include one or more of the following features. In some embodiments, the subject is vertebrate animal or an invertebrate animal. In some embodiments, the subject is a mammalian subject. In some embodiments, the mammalian subject is a human subject. Accordingly, the recombinant polypeptides produced by the method disclosed herein are also within the scope of the disclosure.

Non-limiting exemplary embodiments of the disclosed methods for producing a recombinant polypeptide can include one or more of the following features. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life. In some embodiments of the methods of producing a recombinant polypeptide as described herein, the N-terminus of the produced polypeptide can be further chemically or enzymatically modified to increase half-life. In some embodiments, the C-terminus of the produced polypeptide is chemically or enzymatically modified to increase half-life. Non-limiting examples of chemical and enzymatic modifications suitable for the methods described herein include PEGylation, XTENylation, PASylation®, ELPylation, and HAPylation. Techniques, systems, and reagents suitable for these modifications are known in the art. According, in some embodiments, the polypeptide produced by the methods described herein can be PEGylated, XTENylated, PASylated, ELPylated, and/or HAPylated to increase half-life. In some embodiments the produced polypeptide is conjugated to another protein or peptide (e.g., serum albumin, an antibody Fc domain, transferrin, GLK, or CTP peptide) to increase half-life.

### D. Pharmaceutical Compositions

The nucleic acid constructs, recombinant cells, recombinant RNA molecules, recombinant polypeptides of the disclosure can be incorporated into compositions, including pharmaceutical compositions. Such compositions generally include one or more of the nucleic acid constructs (e.g., vectors or srRNA molecules), recombinant cells, recombinant RNA molecules, recombinant polypeptides described and provided herein, and a pharmaceutically acceptable excipient, e.g., carrier or diluent. In some embodiments, the compositions of the disclosure are formulated for the prevention, treatment, or management of a health condition such as an immune disease or a microbial infection. For example, the compositions of the disclosure can be formulated as a prophylactic composition, a therapeutic composition, or a pharmaceutical composition comprising a pharmaceutically acceptable excipient, or a mixture thereof. In some embodiments, the compositions of the present disclosure are formulated for use as a vaccine. In some embodiments, the compositions of the present application are formulated for use as an adjuvant.

Accordingly, in one aspect, provided herein are pharmaceutical compositions including a pharmaceutically acceptable excipient and: a) a nucleic acid construct (e.g., a vector or srRNA molecule) of the disclosure; b) a recombinant cell of the disclosure; and/or c) a recombinant polypeptide of the disclosure.

Non-limiting exemplary embodiments of the pharmaceutical compositions of the disclosure can include one or more of the following features. In some embodiments, provided herein are compositions including a nucleic acid construct (e.g., a vector or srRNA molecule) as disclosed herein and a pharmaceutically acceptable excipient. In some embodiments, provided herein are compositions including a recombinant cell as disclosed herein and a pharmaceutically acceptable excipient. In some embodiments, provided herein are compositions including a recombinant RNA molecule as disclosed herein and a pharmaceutically acceptable excipient. In some embodiments, the compositions include a recombinant polypeptide of as disclosed herein and a pharmaceutically acceptable excipient. In some embodiments, the nucleic acid constructs of the disclosure (e.g., a vectors or srRNA molecules) can be used in a naked form or formulated with a delivery vehicle. Exemplary delivery vehicles suitable for the compositions and methods of the disclosure include, but are not limited to liposomes (e.g., neutral or anionic liposomes), microspheres, immune stimulating complexes (ISCOMS), lipid-based nanoparticles (LNP), solid lipid nanoparticles (SLN), polyplexes, polymer nanoparticles, viral replicon particles (VRPs), or conjugated with bioactive ligands, which can facilitate delivery and/or enhance the immune response. These compounds are readily available to one skilled in the art; for example, see Liposomes: A Practical Approach, RCP New Ed, IRL press (1990). Adjuvants other than liposomes and the like are also used and are known in the art. Adjuvants may protect the antigen (e.g., nucleic acid constructs, vectors, srRNA molecules) from rapid dispersal by sequestering it in a local deposit, or they may contain substances that stimulate the host to secrete factors that are chemotactic for macrophages and other components of the immune system. An appropriate selection can be made by those skilled in the art, for example, from those described below.

In some embodiments, a composition of the disclosure can include one or more of the following: physiologic buffer, a liposome, a lipid-based nanoparticle (LNP), a solid lipid nanoparticle (SLN), a polyplex, a polymer nanoparticle, a viral replicon particle (VRP), a microsphere, an immune stimulating complex (ISCOM), a conjugate of bioactive ligand, or a combination of any thereof.

The composition of the disclosure can be formulated in a format to be compatible with its intended route of administration, such as liposome, a lipid-based nanoparticle (LNP), or a polymer nanoparticle. Accordingly, in some embodiments, the compositions of the disclosure that formulated in a liposome. In some embodiments, the compositions of the disclosure that formulated in a lipid-based nanoparticle (LNP). LNP are generally less immunogenic than viral particles. While many humans have preexisting immunity to viral particles there is no pre-existing immunity to LNP. In addition, adaptive immune response against LNP is unlikely to occur which enables repeat dosing of LNP.

The lipids suitable for the compositions and methods described herein can be cationic lipids, ionizable cationic lipids, anionic lipids, or neutral lipids.

In some embodiments, the LNP of the disclosure can include one or more ionizable lipids. As used herein, the term “ionizable lipid” refers to a lipid that is cationic or becomes ionizable (protonated) as the pH is lowered below the pKa of the ionizable group of the lipid, but is more neutral at higher pH values. At pH values below the pKa, the lipid is then able to associate with negatively charged nucleic acids (e.g., oligonucleotides). As used herein, the term “ionizable lipid” includes lipids that assume a positive charge on pH decrease from physiological pH, and any of a number of lipid species that carry a net positive charge at a selective pH, such as physiological pH. Permanently cationic lipids such as DOTMA have proven too toxic for clinical use. The ionizable lipid can be present in lipid formulations according to other embodiments, preferably in a ratio of about 30 to about 70 Mol %, in some embodiments, about 30 Mol %, in other embodiments, about 40 Mol %, in other embodiments, about 45 Mol % in other embodiments, about 47.5 Mol % in other embodiments, about 50 Mol %, in still other embodiments, and about 60 Mol % in yet others (“Mol %” means the percentage of the total moles that is of a particular component). The term “about” in this paragraph signifies a plus or minus range of 5 Mol %. DODMA, or 1,2-dioleyloxy-3-dimethylaminopropane, is an ionizable lipid, as is DLin-MC3-DMA or 0-(Z,Z,Z,Z-heptatriaconta-6,9,26,29-tetraen-19-yl)-4-(N,N-dimethylamino) (“MC3”).

Exemplary ionizable lipids suitable for the compositions and methods of the disclosure includes those described in PCT publications WO2020252589A1 and WO2021000041A1, U.S. Pat. Nos. 8,450,298 and 10,844,028, and Love K. T. et al., Proc Natl Acad Sci USA, Feb. 2, 2010 107 (5) 1864-1869, all of which are hereby incorporated by reference in their entirety. Accordingly, in some embodiments, the LNP of the disclosure includes one or more lipid compounds described in Love K. T. et al. (2010 supra), such as C16-96, C14-110, and C12-200. In some embodiments, the LNP includes an ionizable cationic lipid selected from the group consisting of ALC-0315, C12-200, LN16, MC3, MD1, SM-102, and a combination of any thereof. In some embodiments, the LNP of the disclosure includes C12-200 lipid. The structure of C12-200 lipid is known in the art and described in, e.g., U.S. Pat. Nos. 8,450,298 and 10,844,028, which are hereby incorporated by reference in their entirety. In some embodiments the C12-200 is combined with cholesterol, C14-PEG2000, and DOPE. In some embodiments, the C12-200 is combined with DSPC and DMG-PEG2000.

In some embodiments, the LNP of the disclosure includes one or more cationic lipids. Several different ionizable cationic lipids have been developed for use in LNP. Suitable cationic lipids include, but are not limited to, 98N12-5, C12-200, C14-PEG2000, DLin-KC2-DMA (KC2), DLin-MC3-DMA (MC3), XTC, MD1, and 7C1. In one type of LNP, a GalNAc moiety is attached to the outside of the LNP and acts as a ligand for uptake into the liver via the asialyloglycoprotein receptor. Any of these cationic lipids can be used to formulate LNP for delivery of the srRNA constructs and nucleic acid constructs of the disclosure.

In some embodiments, the LNP of the disclosure includes one or more neutral lipids. Non-limiting neutral lipids suitable for the compositions and methods of the disclosure include DPSC, DPPC, POPC, DOPE, and SM. In some embodiments, the LNP of the disclosure includes one or more ionizable lipid compounds described in PCT publications WO2020252589A1 and WO2021000041A1.

A number of other lipids or combination of lipids that are known in the art can be used to produce a LNP. Non-limiting examples of lipids suitable for use to produce LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Additional non-limiting examples of cationic lipids include 98N12-5, C12-200, C14-PEG2000, DLin-KC2-DMA (KC2), DLin-MC3-DMA (MC3), XTC, MD1, 7C1, and a combination of any thereof. Additional non-limiting examples of neutral lipids include DPSC, DPPC, POPC, DOPE, and SM. Non-limiting examples of PEG-modified lipids include PEG-DMG, PEG-CerC14, and PEG-CerC20.

In some embodiments, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In some embodiments, the lipid-based nanoparticles have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

In some embodiments, the compositions of the disclosure that formulated in a polymer nanoparticle. In some embodiments, the compositions are immunogenic compositions, e.g., composition that can stimulate an immune response in a subject. In some embodiments, the immunogenic compositions are formulated as a vaccine. In some embodiments, the pharmaceutical compositions are formulated as an adjuvant. In some embodiments, the immunogenic compositions are formulated as a biotherapeutic e.g., vehicle for gene delivery of different molecules with bioactivity. Non-limiting examples of biotherapeutic include cytokines, chemokines, and other soluble immunomodulators, enzymes, peptide and protein agonists, peptide and protein antagonists, hormones, receptors, antibodies and antibody-derivatives, growth factors, transcription factors, and gene silencing/editing molecules. In some embodiments, the pharmaceutical compositions are formulated as an adjuvant. In some embodiments, the compositions are non-immunogenic or minimally immunogenic (e.g. compositions that minimally stimulate an immune response in a subject). In some embodiments, the non-immunogenic or minimally immunogenic compositions are formulated as a biotherapeutic.

In some embodiments, the immunogenic compositions are substantially non-immunogenic to a subject. In some embodiments, the pharmaceutical compositions are formulated for one or more of intranasal administration, transdermal administration, intraperitoneal administration, intramuscular administration, intratracheal administration, intranodal administration, intratumoral administration, intraarticular administration, intravenous administration, subcutaneous administration, intravaginal administration, intraocular, rectal, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In these cases, the composition should be sterile and should be fluid to the extent that easy syringeability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the pharmaceutical compositions of the disclosure are formulated for inhalation, such as an aerosol, spray, mist, liquid, or powder. Administration by inhalation may be in the form of either dry powders or aerosol formulations, which are inhaled by a subject (e.g., a patient) either through use of an inhalation device, e.g., a microspray, a pressurized metered dose inhaler, or nebulizer.

In some embodiments, the composition is formulated for one or more of intranasal administration, transdermal administration, intramuscular administration, intranodal administration, intravenous administration, intraperitoneal administration, oral administration, intravaginal, intratumoral administration, subcuteaneous administration, intraarticular administration, or intra-cranial administration. In some embodiments, the administered composition results in a modulated (e.g., increased or decreased) production of interferon in the subject.

### Methods of the Disclosure

Administration of any one of the therapeutic compositions described herein, e.g., nucleic acid constructs (e.g., vectors or srRNA molecules), recombinant cells, recombinant RNA molecules, recombinant polypeptides, and/or pharmaceutical compositions, can be useful in the treatment and/or prevention of relevant health conditions, such as proliferative disorders (e.g., cancers), infectious diseases (e.g., acute infections, chronic infections, or viral infections), rare diseases, and/or autoimmune diseases, and/or inflammatory diseases. In some embodiments, the nucleic acid constructs (e.g., vectors or srRNA constructs), recombinant cells, recombinant RNA molecules, recombinant polypeptides, and/or pharmaceutical compositions as described herein can be useful for modulating, e.g., eliciting or suppressing, an immune response in a subject in need thereof. In some embodiments, the nucleic acid constructs (e.g., vectors or srRNA molecules), recombinant cells, recombinant RNA molecules, recombinant polypeptides, and/or pharmaceutical compositions as described herein can be incorporated into therapeutic agents for use in methods of treating a subject who has, who is suspected of having, or who may be at high risk for developing one or more relevant health conditions or diseases. Exemplary health conditions or diseases can include, without limitation, cancers, immune diseases, autoimmune diseases, inflammatory diseases, gene therapy, gene replacement, cardiovascular diseases, age-related pathologies, rare disease, acute infection, and chronic infection. In some embodiments, the subject is a patient under the care of a physician.

Examples of autoimmune diseases suitable for the methods of the disclosure include, but are not limited to, rheumatoid arthritis, osteoarthritis, Still's disease, Familiar Mediterranean Fever, systemic sclerosis, multiple sclerosis, ankylosing spondylitis, Hashimoto's thyroiditis, systemic lupus erythematosus, Sjogren's syndrome, diabetic retinopathy, diabetic vasculopathy, diabetic neuralgia, insulitis, psoriasis, alopecia areata, warm and cold autoimmune hemolytic anemia (AIHA), pernicious anemia, acute inflammatory diseases, autoimmune adrenalitis, chronic inflammatory demyelinating polyneuropathy (CIDP), Lambert-Eaton syndrome, lichen sclerosis, Lyme disease, Graves disease, Behçet's disease, Ménière's disease, reactive arthritis (Reiter's syndrome), Churg-Strauss syndrome, Cogan syndrome, CREST syndrome, pemphigus vulgaris and pemphigus foliaceus, bullous pemphigoid, polymyalgia rheumatica, polymyositis, primary biliary cirrhosis, pancreatitis, peritonitis, psoriatic arthritis, rheumatic fever, sarcoidosis, Sjörgensen syndrome, scleroderma, celiac disease, stiff-man syndrome, Takayasu arteritis, transient gluten intolerance, autoimmune uveitis, vitiligo, polychondritis, dermatitis herpetiformis (DH) or Duhring's disease, fibromyalgia, Goodpasture syndrome, Guillain-Barré syndrome, Hashimoto thyroiditis, autoimmune hepatitis, inflammatory bowel disease (IBD), Crohn's disease, colitis ulcerosa, myasthenia gravis, immune complex disorders, glomerulonephritis, polyarteritis nodosa, anti-phospholipid syndrome, polyglandular autoimmune syndrome, idiopathic pulmonary fibrosis, idiopathic thrombocytopenia purpura (ITP), urticaria, autoimmune infertility, juvenile rheumatoid arthritis, sarcoidosis, and autoimmune cardiomyopathy.

Non-limiting examples of infection suitable for the methods of the disclosure include infections with viruses such as human immunodeficiency virus (HIV), hepatitis B virus (HBV), hepatitis B virus (HCV), Cytomegalovirus (CMV), respiratory syncytial virus (RSV), human papillomavirus (HPV), Epstein-Barr virus (EBV), severe acute respiratory syndrome coronavirus 2 (SARS-COV2), severe acute respiratory syndrome coronavirus (SARS-COV), Middle East Respiratory Syndrome (MERS), influenza virus, and Ebola virus. Additional infections suitable for the methods of the disclosure include infections with intracellular parasites such as Leishmania, Rickettsia, Chlamydia, Coxiella, Plasmodium, Brucella, mycobacteria, Listeria, Toxoplasma and Trypanosoma.

In some embodiments, the nucleic acid constructs (e.g., vectors or srRNA molecules), recombinant cells, recombinant RNA molecules, recombinant polypeptides, and/or pharmaceutical compositions, can be useful in the treatment and/or prevention of immune diseases, autoimmune diseases, or inflammatory diseases such as, for example, glomerulonephritis, inflammatory bowel disease, nephritis, peritonitis, psoriatic arthritis, osteoarthritis, Still's disease, Familiar Mediterranean Fever, systemic scleroderma and sclerosis, inflammatory bowel disease (IBD), Crohn's disease, ulcerative colitis, acute lung injury, meningitis, encephalitis, uveitis, multiple myeloma, glomerulonephritis, nephritis, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, hemolytic anemia, myasthenia gravis, lupus nephritis, lupus erythematosus, rheumatoid arthritis (RA), ankylosing spondylitis, pemphigus, Graves' disease, Hashimoto's thyroiditis, small vessel vasculitis, Omen's syndrome, chronic renal failure, autoimmune thyroid disease, acute infectious mononucleosis, HIV, herpes virus associated diseases, human virus infections, coronavirus, other enterovirus, herpes virus, influenza virus, parainfluenza virus, respiratory syncytial virus or adenovirus infection, bacteria pneumonia, wounds, sepsis, cerebral stroke/cerebral edema, ischaemia-reperfusion injury, and hepatitis C.

Non-limiting examples of inflammatory suitable for the methods of the disclosure include inflammatory diseases such as asthma, inflammatory bowel disease (IBD), chronic colitis, splenomegaly, and rheumatoid arthritis.

Accordingly, in one aspect of the disclosure, provided herein are methods for modulating an immune response in a subject in need thereof, the method includes administering to the subject a composition including one or more of the following: a) a nucleic acid construct of the disclosure; b) a recombinant RNA molecule of the disclosure; c) a recombinant cell of the disclosure; d) a recombinant polypeptide of the disclosure; and e) a pharmaceutical composition of the disclosure.

In another aspect, provided herein are methods for preventing and/or treating a health condition in a subject in need thereof, the method includes prophylactically or therapeutically administering to the subject a composition including one or more of the following: a) a nucleic acid construct of the disclosure; b) a recombinant RNA molecule of the disclosure; c) a recombinant cell of the disclosure; d) a recombinant polypeptide of the disclosure; and e) a pharmaceutical composition of any one of the disclosure.

In some embodiments, the health condition is a proliferative disorder or a microbial infection (e.g., bacterial infection, micro-fungal infection, or viral infection). In some embodiments, the subject has or is suspected of having a condition associated with proliferative disorder or a microbial infection (e.g., bacterial infection, micro-fungal infection, or viral infection).

In some embodiments, the health condition is a rare disease, e.g., a disease or condition that affects less than 200,000 people in the United States, as defined by The Orphan Drug Act (www.fda.gov/patients/rare-diseases-fda) and/or an inflammatory and/or autoimmune disorder. In some embodiments, the subject has or is suspected of having a condition associated with an inflammatory and/or autoimmune disorder and/or a rare disease (e.g. including but not limited to Familial Mediterranean Fever or adult onset Still's disease).

In some embodiments, the disclosed composition is formulated to be compatible with its intended route of administration. For example, the nucleic acid constructs, recombinant cells, recombinant RNA molecules, recombinant polypeptides, and/or pharmaceutical compositions of the disclosure may be given orally or by inhalation, but it is more likely that they will be administered through a parenteral route. Examples of parenteral routes of administration include, for example, intravenous, intranodal, intradermal, intratumoral, intraarticular, subcutaneous, transdermal (topical), transmucosal, intravaginal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject nucleic acid constructs, recombinant cells, recombinant RNA molecules, recombinant polypeptides, and/or pharmaceutical compositions of the disclosure can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

For example, the data obtained from the cell culture assays and animal studies can be used in formulating a range of dosage for use in humans. The dosage of such compounds lies generally within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutic compositions described herein, e.g., nucleic acid constructs, recombinant cells, recombinant RNA molecules, recombinant polypeptides, and/or pharmaceutical compositions, can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject multivalent polypeptides and multivalent antibodies of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours. With regard to nucleic acid constructs, recombinant RNA molecules, and recombinant polypeptides, the therapeutically effective amount of a nucleic acid construct, recombinant RNA molecule, or recombinant polypeptide of the disclosure (e.g., an effective dosage) depends on the nucleic acid construct, recombinant RNA molecule, or recombinant polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, one, two, three, four, or more nucleic acid constructs, recombinant cells, recombinant RNA molecules, or recombinant polypeptides of the disclosure can be used in combination.

As discussed supra, a therapeutically effective amount in some embodiments can be an amount of a therapeutic composition that is sufficient to promote a particular effect when administered to a subject, such as one who has, is suspected of having, or is at risk for a health condition, e.g., a disease or infection. In some embodiments, an effective amount includes an amount sufficient to prevent or delay the development of a symptom of the disease or infection, alter the course of a symptom of the disease or infection (for example but not limited to, slow the progression of a symptom of the disease or infection), or reverse a symptom of the disease or infection. It is understood that for any given case, an appropriate effective amount can be determined by one of ordinary skill in the art using routine experimentation.

The efficacy of a treatment including a disclosed therapeutic composition for the treatment of disease or infection can be determined by the skilled clinician. However, a treatment is considered effective treatment if at least any one or all of the signs or symptoms of disease or infection are improved or ameliorated. Efficacy can also be measured by failure of an individual to worsen as assessed by hospitalization or need for medical interventions (e.g., progression of the disease or infection is halted or at least slowed). Methods of measuring these indicators are known to those of skill in the art and/or described herein. Treatment includes any treatment of a disease or infection in a subject or an animal (some non-limiting examples include a human, or a mammal) and includes: (1) inhibiting the disease or infection, e.g., arresting, or slowing the progression of symptoms; or (2) relieving the disease or infection, e.g., causing regression of symptoms; and (3) preventing or reducing the likelihood of the development of symptoms.

In some embodiments, the nucleic acid constructs, recombinant cells, recombinant RNA molecules, recombinant polypeptides, and/or pharmaceutical compositions of the disclosure can be administered to a subject in a composition having a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject. In some embodiments, the administered composition results in an increased production of interferon in the subject by at least 30%, at least 40%, at least 50%, at least 60%, at least 70%, at least 80%, at least 90%, at least 95%, or at least 100% as compared to interferon production in a subject that has not been administered with the composition. In some embodiments of the disclosed methods, the subject is a vertebrate animal or an invertebrate animal. In some embodiments, the subject is a mammalian subject. In some embodiments, the mammalian subject is a human subject.

As described above, pharmaceutically acceptable carriers suitable for injectable use include sterile aqueous solutions (where water soluble) or dispersions and sterile powders for the extemporaneous preparation of sterile injectable solutions or dispersions. In these cases, the composition must be sterile and must be fluid to the extent that easy syringeability exists. The composition must further be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, asorbic acid, thimerosal, and the like.

Sterile injectable solutions can be prepared by incorporating the nucleic acid constructs, recombinant cells, and/or recombinant polypeptides in the required mount in an appropriate solvent with one or a combination of ingredients enumerated above, as required, followed by filtered sterilization.

When the nucleic acid constructs, recombinant cells, recombinant RNA molecules, recombinant polypeptides, and/or pharmaceutical compositions as described herein are suitably protected, as described above, they may be orally administered, for example, with an inert diluent or an assimilable edible carrier. The nucleic acid constructs, recombinant cells, recombinant RNA molecules, recombinant polypeptides, and/or pharmaceutical compositions and other ingredients may also be enclosed in a hard or soft shell gelatin capsule, compressed into tablets, or incorporated directly into the individual's diet. For oral therapeutic administration, the active compound may be incorporated with excipients and used in the form of ingestible tablets, buccal tablets, troches, capsules, elixirs, suspensions, syrups, wafers, and the like.

In some embodiments, the nucleic acid constructs, recombinant RNA molecules, and recombinant polypeptides of the disclosure can be delivered to a cell or a subject by a lipid-based nanoparticle (LNP). While many humans have preexisting immunity to viral particles there is no pre-existing immunity to LNP. In addition, adaptive immune response against LNP is unlikely to occur which enables repeat dosing of LNP.

Several different ionizable cationic lipids have been developed for use in LNP. Non-limiting examples of ionizable cationic lipids include C12-200, MC3, LN16, and MD1 among others. For example, in one type of LNP, a GalNAc moiety is attached to the outside of the LNP and acts as a ligand for uptake into the liver via the asialyloglycoprotein receptor. Any of these cationic lipids can be used to formulate LNP for delivery of the nucleic acid constructs and recombinant polypeptides of the disclosure to the liver.

In some embodiments, a LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also have hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20.

In some embodiments, the lipids can be combined in any number of molar ratios to produce a LNP. In addition, the polynucleotide(s) can be combined with lipid(s) in a wide range of molar ratios to produce a LNP.

In some embodiments, the therapeutic compositions described herein, e.g., nucleic acid constructs, recombinant cells, recombinant RNA molecules, recombinant polypeptides, and/or pharmaceutical compositions are incorporated into therapeutic compositions for use in methods of preventing or treating a subject who has, who is suspected of having, or who may be at high risk for developing a cancer, an autoimmune disease, and/or an infection.

In some embodiments, the therapeutic compositions described herein, e.g., nucleic acid constructs, recombinant cells, recombinant RNA molecules, recombinant polypeptides, and/or pharmaceutical compositions are incorporated into therapeutic compositions for use in methods of preventing or treating a subject who has, who is suspected of having, or who may be at high risk for developing a microbial infection. In some embodiments, the microbial infection is a bacterial infection. In some embodiments, the microbial infection is a fungal infection. In some embodiments, the microbial infection is a viral infection.

### Additional Therapies

In some embodiments, a composition according to the present disclosure is administered to the subject individually as a single therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein as well as written instructions for making and using the same. In particular, some embodiments of the disclosure provide kits for modulating an immune response in a subject. Some other embodiments relate to kits for the prevention of a health condition in a subject in need thereof. Some other embodiments relate to kits for methods of treating a health condition in a subject in need thereof. For example, provided herein, in some embodiments, are kits that include one or more of the nucleic acid constructs (e.g., vectors and srRNA molecules), recombinant cells, recombinant RNA molecules, recombinant polypeptides, and/or pharmaceutical compositions as provided and described herein, as well as written instructions for making and using the same.

In some embodiments, the kits of the disclosure further include one or more means useful for the administration of any one of the provided nucleic acid constructs (e.g., vectors and srRNA molecules), recombinant cells, recombinant RNA molecules, recombinant polypeptides, and/or pharmaceutical compositions to a subject. For example, in some embodiments, the kits of the disclosure further include one or more syringes (including pre-filled syringes) and/or catheters (including pre-filled syringes) used to administer any one of the provided nucleic acid constructs (e.g., vectors and srRNA molecules), recombinant cells, recombinant RNA molecules, recombinant polypeptides, and/or pharmaceutical compositions to a subject. In some embodiments, a kit can have one or more additional therapeutic agents that can be administered simultaneously or sequentially with the other kit components for a desired purpose, e.g., for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative controls, positive controls, reagents suitable for in vitro production of the provided nucleic acid constructs, recombinant cells, recombinant polypeptides, and/or pharmaceutical compositions of the disclosure.

In some embodiments, the components of a kit can be in separate containers. In some other embodiments, the components of a kit can be combined in a single container. Accordingly, in some embodiments of the disclosure, the kit includes one or more of the nucleic acid constructs (e.g., vectors and srRNA molecules), recombinant cells, recombinant RNA molecules, recombinant polypeptides, and/or pharmaceutical compositions as provided and described herein in one container (e.g., in a sterile glass or plastic vial) and a further therapeutic agent in another container (e.g., in a sterile glass or plastic vial).

In another embodiment, the kit includes a combination of the compositions described herein, including one or more nucleic acid constructs, recombinant cells, recombinant RNA molecules, and/or recombinant polypeptides of the disclosure in combination with one or more further therapeutic agents formulated together, optionally, in a pharmaceutical composition, in a single, common container.

If the kit includes a pharmaceutical composition for parenteral administration to a subject, the kit can include a device (e.g., an injection device or catheter) for performing such administration. For example, the kit can include one or more hypodermic needles or other injection devices as discussed above containing one or more nucleic acid constructs, recombinant cells, recombinant RNA molecules, and/or recombinant polypeptides of the disclosure.

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). POR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### Construction of Modified Alphavirus Vectors

This Example describes the results of experiments performed to construct a number of base alphavirus vectors (e.g., without a heterologous gene) that were subsequently used for expression of a gene of interest (e.g., a hemagglutinin (HA) gene from influenza).

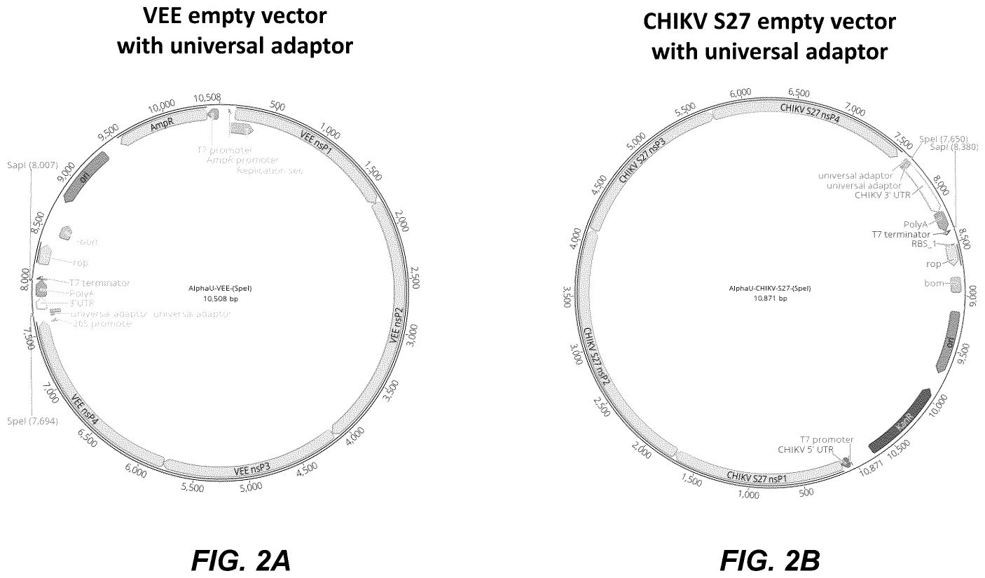

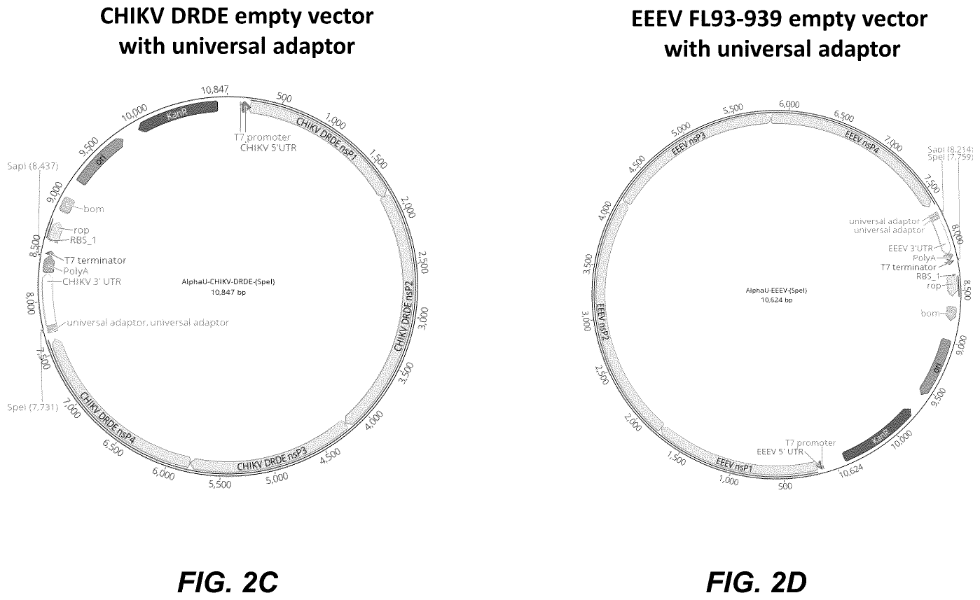

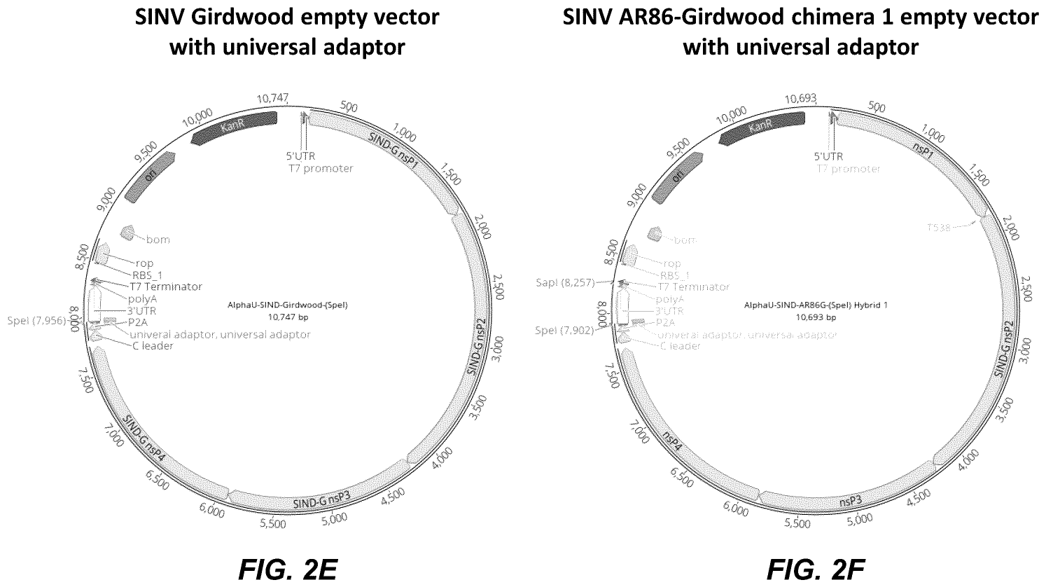

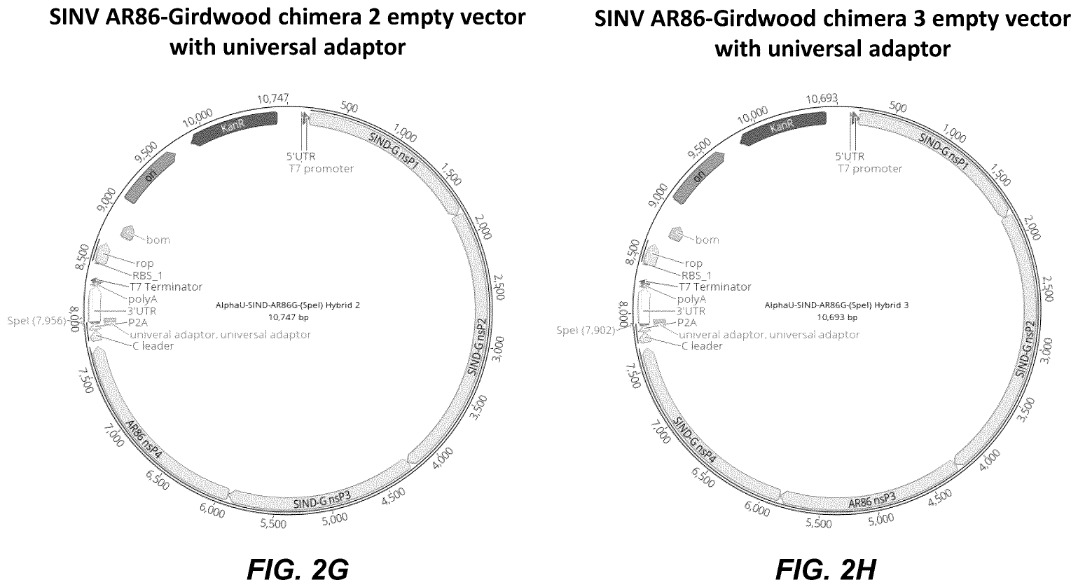

The VEE empty vector with the universal adaptor (FIG. 2A) was constructed by PCR amplification from a VEE TC-83 replicon (Genbank L01443) flanked by a 5′ bacteriophage T7 RNA polymerase promoter (5′-TAATACGACTCACTATAG-3′; SEQ ID NO: 28) and a 3′ 38 residue poly(A) followed by a T7 terminator sequence (5′-AACCCCTCTCTAAACGGAGGGGTTTTTTT-3′; SEQ ID NO: 29) followed by a downstream NotI site in a pYL plasmid backbone with a synthetic forward primer containing the universal adaptor sequence containing the SpeI site (5′-CTGGAGACGTGGAGGAGAACCCTGGACCTACTAGTGACCGCTACGCCCCAATGACC CGACCAGC-3′) and a synthetic reverse primer to generate a PCR product with 30 bp of homology on the ends and was circularized by Gibson Assembly® procedure. A silent mutation A2087G was made to eliminate a SpeI site in nsP2. This product has the universal adaptor in place of the structural gene. A synthetic DNA fragment with 30 bp homology flanks containing the SapI site downstream of the poly(A) with 30 bp homology ends was inserted into the product linearized by digestion with SpeI and NotI to generate the final vector.

The CHIKV S27 empty vector with the universal adaptor (FIG. 2B) was constructed by PCR amplification from a CHIKV S27 replicon (Genbank AF369024) flanked by a 5′ bacteriophage T7 RNA polymerase promoter (5′-TAATACGACTCACTATAG-3′; SEQ ID NO: 28) and a 3′ 37 residue poly(A) followed by a T7 terminator sequence (5′-AACCCCTCTCTAAACGGAGGGGTTTTTTT-3′; SEQ ID NO: 29) followed by a downstream NotI site in a pYL plasmid backbone with a synthetic forward primer containing the universal adaptor sequence containing the SpeI site (5′-CTGGAGACGTGGAGGAGAACCCTGGACCTACTAGTGACCGCTACGCCCCAATGACC CGACCAGC-3′; SEQ ID NO: 20) and a synthetic reverse primer to generate a PCR product with 30 bp of homology on the ends and was circularized by Gibson Assembly® procedure. This product has the universal adaptor in place of the structural gene. A synthetic DNA fragment with 30 bp homology flanks containing the SapI site downstream of the poly(A) with 30 bp homology ends was inserted into the product linearized by digestion with SpeI and NotI to generate the final vector.

The CHIKV DRDE empty vector with the universal adaptor (FIG. 2C) was constructed by PCR amplification from a CHIKV DRDE replicon (Genbank EF210157) with a CHIKV S27 3′ UTR (Genbank AF369024) flanked by a 5′ bacteriophage T7 RNA polymerase promoter (5′-TAATACGACTCACTATAG-3′; SEQ ID NO: 28) and a 3′ 37 residue poly(A) followed by a T7 terminator sequence (5′-AACCCCTCTCTAAACGGAGGGGTTTTTTT-3′; SEQ ID NO: 29) followed by a downstream NotI site in a pYL plasmid backbone with a synthetic forward primer containing the universal adaptor sequence containing the SpeI site (5′-CTGGAGACGTGGAGGAGAACCCTGGACCTACTAGTGACCGCTACGCCCCAATGACC CGACCAGC-3′; SEQ ID NO: 20) and a synthetic reverse primer to generate a PCR product with 30 bp of homology on the ends and was circularized by Gibson Assembly® procedure. This product has the universal adaptor in place of the structural gene. A synthetic DNA fragment with 30 bp homology ends containing the SapI site downstream of the poly(A) with 30 bp homology was inserted into the product linearized by digestion with SpeI and NotI to generate the final vector.

The EEEV FL93-939 empty vector with the universal adaptor (FIG. 2D) was constructed by PCR amplification from a EEEV FL93-939 replicon (Genbank EF151502) flanked by a 5′ bacteriophage T7 RNA polymerase promoter (5′-TAATACGACTCACTATAG-3′; SEQ ID NO: 28) and a 3′ 37 residue poly(A) followed by a T7 terminator sequence (5′-AACCCCTCTCTAAACGGAGGGGTTTTTTT-3′; SEQ ID NO: 29) followed by a downstream NotI site in a pYL plasmid backbone with a synthetic forward primer containing the universal adaptor sequence containing the SpeI site (5′-CTGGAGACGTGGAGGAGAACCCTGGACCTACTAGTGACCGCTACGCCCCAATGACC CGACCAGC-3′; SEQ ID NO: 20) and a synthetic reverse primer to generate a PCR product with 30 bp of homology on the ends and was circularized by Gibson Assembly® procedure. A silent mutation A3550C was made to eliminate a SpeI site in nsP2. Silent mutations G301A, G4516A, and G7399 were made to eliminate SapI sites in nsP1, nsP3, and nsP4 respectively. This product has the universal adaptor in place of the structural gene. A synthetic DNA fragment with 30 bp homology ends containing the SapI site downstream of the poly(A) with 30 bp homology was inserted into the product linearized by digestion with SpeI and NotI to generate the final vector.

The SINV Girdwood empty vector with universal adaptor (SEQ ID NO: 27) (FIG. 2E) was constructed by PCR amplification from a SINV Girdwood replicon (Genbank MF459683) flanked by a 5′ bacteriophage T7 RNA polymerase promoter (5′-TAATACGACTCACTATAG-3′; SEQ ID NO: 28) and a 3′ 37 residue poly(A) followed by a T7 terminator sequence (5′-AACCCCTCTCTAAACGGAGGGGTTTTTTT-3′; SEQ ID NO: 29) followed by a downstream NotI site in a pYL plasmid backbone with a synthetic forward primer containing the universal adaptor sequence containing the SpeI site (5′-CTGGAGACGTGGAGGAGAACCCTGGACCTACTAGTGACCGCTACGCCCCAATGACC CGACCAGC-3′; SEQ ID NO: 20) and a synthetic reverse primer to generate a PCR product with 30 bp of homology on the ends and was circularized by Gibson Assembly® procedure. This product has the universal adaptor in place of the structural gene. A silent mutation A5420G was made to eliminate a SapI site in Girdwood nsP3. A synthetic DNA fragment with 30 bp homology ends containing the SapI site downstream of the poly(A) with 30 bp homology was inserted into the product linearized by digestion with SpeI and NotI to generate the final vector.

The SINV AR86-Girdwood chimera empty vectors with universal adaptors (FIG. 2F-I) were constructed by PCR amplification of the SINV Girdwood empty vector (FIG. 2E) to generate products with 30 bp homology ends to PCR products amplified from an AR86 sequence (Genbank U38305). The fragments were combined by Gibson Assembly® procedure to generate the final vectors. For chimera 1 (FIG. 2F), the Girdwood nsP1, nsP3, and nsP4 were replaced by AR86 nsP1, nsP3, and nsP4 respectively. A silent mutation A5366G was made to eliminate a SapI site in AR86 nsP3. For chimera 2 (FIG. 2G), the Girdwood nsP4 was replaced by AR86 nsP4. For chimera 3 (FIG. 2H), the Girdwood nsP3 was replaced by AR86 nsP3. A silent mutation A5366G was made to eliminate a SapI site in AR86 nsP3. For chimera 4 (FIG. 2I), the Girdwood nsP1 was replaced by AR86 nsP1. The sequences of chimera 1-4 are provided in SEQ ID NOS: 22-25.

### Example 2

### Construction of Modified Alphavirus Vectors with a Gene of Interest

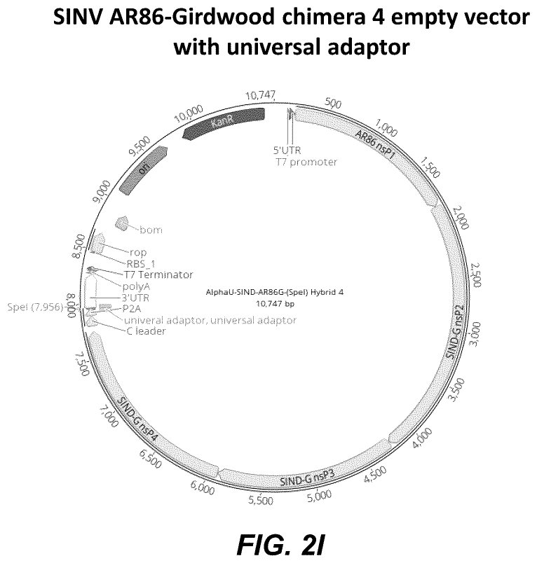

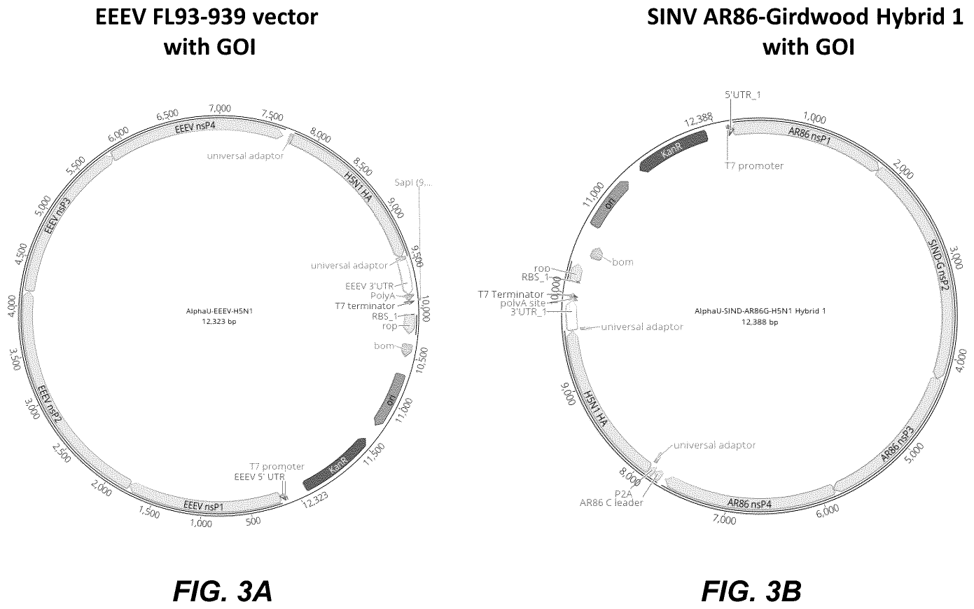

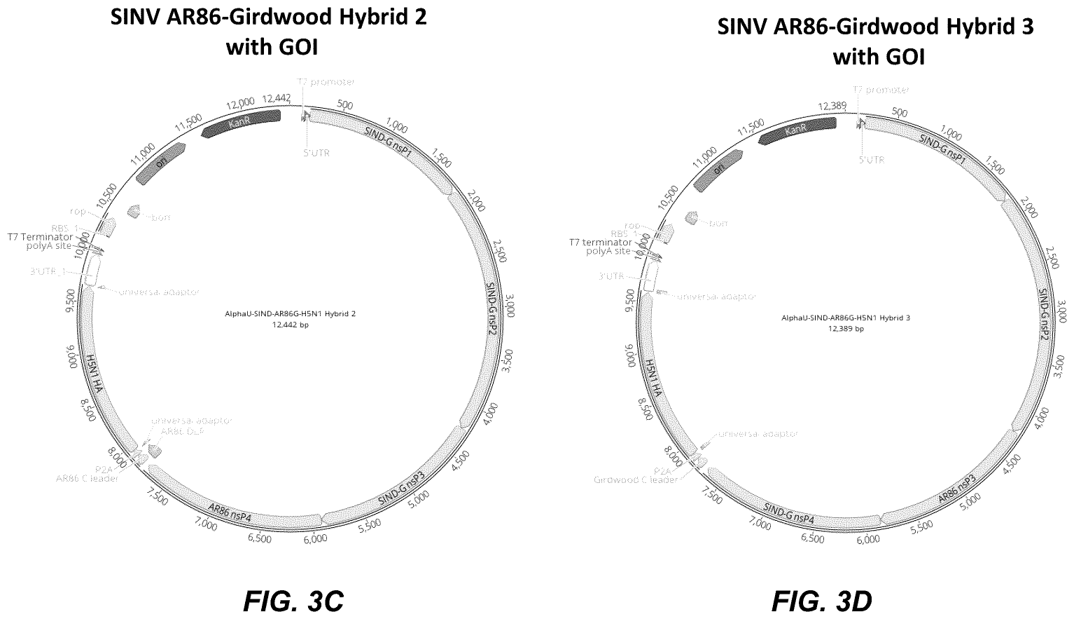

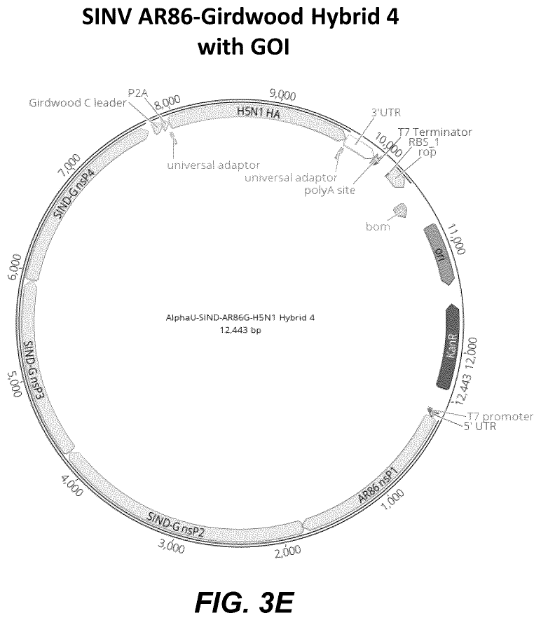

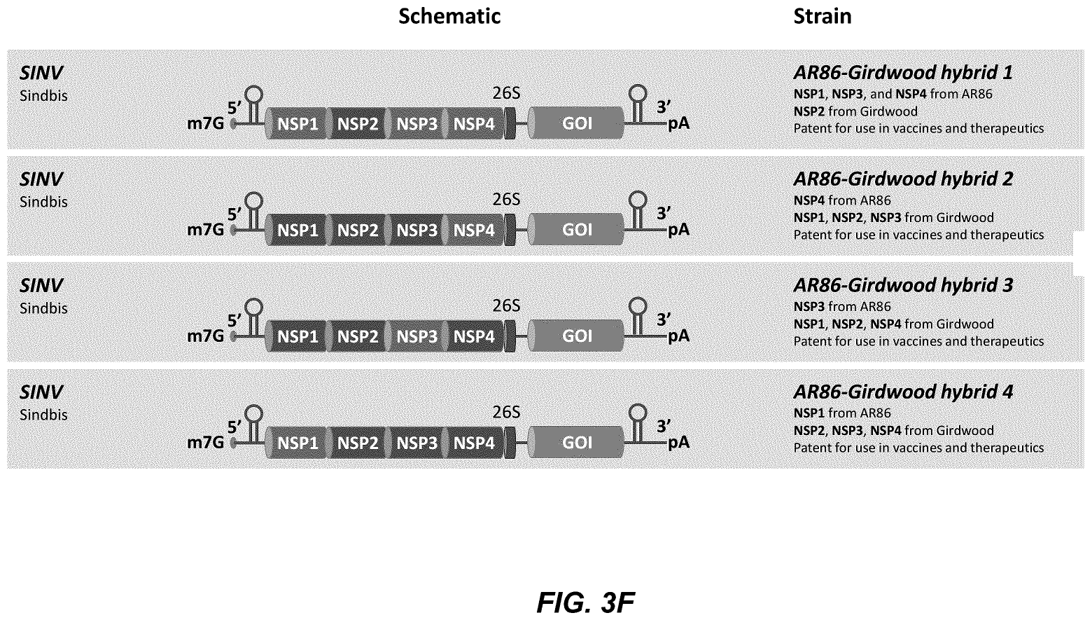

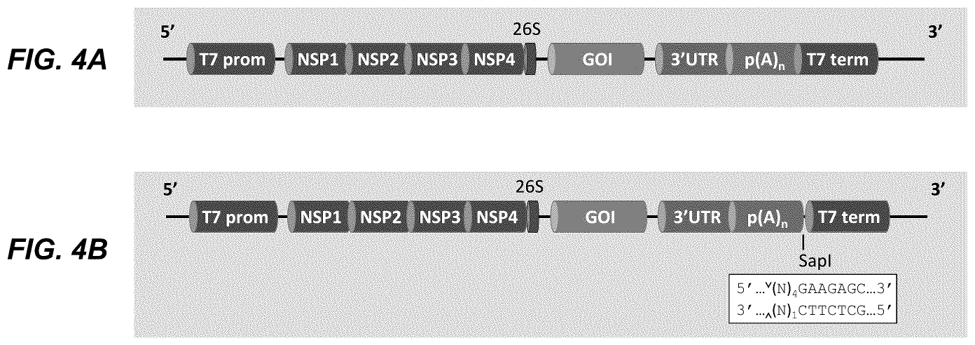

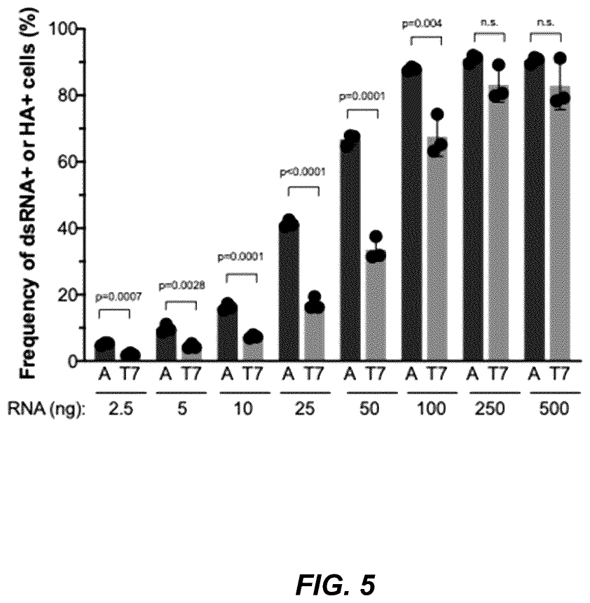

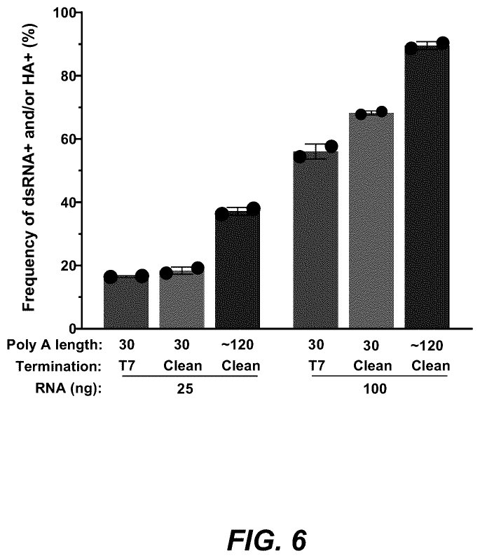

The alphavirus vector in FIG. 3A was constructed by linearization of the empty EEEV universal vector in FIG. 2 by SpeI digestion. The hemagglutinin (HA) gene from influenza (Genbank AY651334) was codon refactored for human expression in silico and synthesized (IDT). The synthetic product was amplified using the following primers which add the universal adaptors as 30 bp homology ends to the PCR product.

Forward primer
(5′-GCTGGAGACGTGGAGGAGAACCCTGGACCTATGGAGAAAATAGTG
CTTCTTTTTG-3′; SEQ ID NO: 30).
Reverse primer
(5′-GCTGGTCGGGTCATTGGGGCGTAGCGGTCAAATGCAAATTCTGCA
TTGTAACG-3′; SEQ ID NO: 31),

The digest product and the PCR product were combined by Gibson Assembly® procedure to result in the final vectors.

The alphavirus vectors in FIGS. 3B-E were constructed from a plasmid containing the SINV Girdwood (Genbank MF459683) replicon encoding the HA gene. For chimera 1 (FIG. 3B) the nsp1, nsP3, nsP4 genes were replaced with the AR86 nsp1, nsp3, and nsP4 genes (Genbank U38305). For chimera 2 (FIG. 3C) the nsP4 gene was replaced with the AR86 nsP4 gene. For chimera 3 (FIG. 3D) the nsP3 gene was replaced with the AR86 nsP3 gene. For chimera 4 (FIG. 3E) the nsP1 gene was replaced with the AR86 nsP1 gene. The replacements were conducted by amplification of PCR products with 30 bp homology ends and combined by Gibson Assembly® procedure. It was observed that no constructs that contained an AR86 nsP2 gene were able to replicate.

### Example 3

### Construction of Modified Alphavirus Vectors with a Lengthened Poly(A)

The VEE empty vector (FIG. 2A) was linearized with SapI and NotI, and a synthetic DNA fragment containing a poly(A) sequence with 170 A residues, followed by a SapI site, a T7 terminator, and 30 bp homology to the linearized empty vector were combined by Gibson Assembly® procedure. A product was isolated with approximately ˜120 As, determined by Sanger sequencing.

### Example 4

### Assessing Minimum Free Energy (MFE) of the 5′ Flanking Domain and 3′ Flanking Domain

The minimum free energy (MFE) structures of the 5′ and 3′ flanking domains and their ΔG values were generated in silico by using the Mfold tool for MFE RNA structure prediction and ΔG calculation (www.unafold.org/, https://doi.org/10.1093/nar/gkg595).

### Example 5

### In Vitro Evaluation of Modified Alphavirus Vectors

This Example describes the results of in vitro experiments performed to evaluate expression levels of the modified alphavirus vector constructs described in Examples 1 and 2 and 3 above, and to investigate any differential behavior thereof (e.g., replication and protein expression).

### List of Vectors:

VEE replicon with universal adaptors, CHIKV S27 replicon with universal adaptors, CHIKV DRDE replicon with universal adaptors, EEEV FL93-939 replicon with universal adaptors, SINV Girdwood, SINV AR86/Girdwood chimeric replicons, VEE replicon with universal adaptors and exclusively adenylate residues in the poly(A), and VEE replicon with universal adaptors and exclusively adenylate residues in the long poly(A).

### Assays:

### In Vitro Transcription:

RNA is prepared by in vitro transcription using a plasmid DNA template linearized by enzymatic digestion. In these examples, the DNA is either linearized with NotI, which cuts downstream of the T7 terminator, or linearized with SapI, which cuts at the end of the poly(A). Bacteriophage T7 polymerase is used for in vitro transcription with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). RNA is purified using phenol/chloroform extraction, or column purification (Monarch® RNA Cleanup Kit, NEB). RNA concentration is determined by absorbance at 260 nm (Nanodrop, Thermo Fisher Scientific).

### Replication:

RNA is transformed by electroporation into BHK-21 or Vero cells (e.g. 4D)-Nucleofector™, Lonza). At 17-20 h following transformation, the cells are fixed and permeabilized (eBioscience™ Foxp3/Transcription Factor Staining Buffer Set, Invitrogen) and stained using a PE-conjugated anti-dsRNA mouse monocolonal antibody (J2, Scicons) to quantify the frequency of dsRNA+ cells and the mean fluorescence intensity (MFI) of dsRNA in individual cells by fluorescence flow cytometry.

### Protein Expression:

RNA is transformed by electroporation into BHK-21 or Vero cells (e.g. 4D)-Nucleofector™, Lonza). At 18-20 h following transformation, the cells were fixed and permeabilized (eBioscience™ Foxp3/Transcription Factor Staining Buffer Set, Invitrogen) and stained using an APC-conjugated anti-HA mouse monoclonal antibody (2B7, Abcam) to quantify the frequency of HA protein+ cells and the mean fluorescence intensity (MFI) of the HA protein in individual cells by fluorescence flow cytometry.

### Additional Experiments:

BHK-21 or Vero cells are pre-treated with a titrated curve of recombinant IFN prior to electroporation of RNA, and impacts on replication and protein expression for each vector are measured using the above assays.

### Example 6

### In Vivo Evaluation of Modified Alphavirus Vectors

This Example describes the results of in vivo experiments performed to evaluate any differential immune responses following vaccination with the modified alphavirus vector constructs described in Examples 1 and 2 and 3 above (e.g., both unformulated and LNP formulated vectors).

### List of Vectors:

VEE replicon with universal adaptors, CHIKV S27 replicon with universal adaptors, CHIKV DRDE replicon with universal adaptors, EEEV FL93-939 replicon with universal adaptors, SINV Girdwood, SINV AR86/Girdwood chimeric replicons, VEE replicon with universal adaptors and exclusively adenylate residues in the poly(A), and VEE replicon with universal adaptors and exclusively adenylate residues in the long poly(A)

### Assays:

### Mice and Injections.

Female C57BL/6 or BALB/c mice are purchased from Charles River Labs or Jackson Laboratories. On day of dosing, between 0.1-10 μg of material is injected intramuscularly split into both quadricep muscles. Vectors are administered either unformulated in saline, or LNP-formulated. Animals are monitored for body weight and other general observations throughout the course of the study. For immunogenicity studies, animals are dosed on Day 0 and Day 21. Spleens were collected at Day 35, and serum was isolated at Days 0, 14, and 35. For protein expression studies, animals are dosed on Day 0, and bioluminescence is assessed on Days 1, 3, and 7. In vivo imaging of luciferase activity is done using an IVIS system at the indicated time points.

### LNP Formulation.

Replicon RNA is formulated in lipid nanoparticles using a microfluidics mixer and analyzed for particle size, polydispersity using dynamic light scattering and encapsulation efficiency. Molar ratios of lipids used in formulating LNP particles is 30% C12-200, 46.5% Cholesterol, 2.5% PEG-2K and 16% DOPE.

### ELISpot.

To measure the magnitude of Influenza-specific T cell responses, IFNγ ELISpot analysis is performed using Mouse IFNγ ELISpot PLUS Kit (HRP) (MabTech) as per manufacturer's instructions. In brief, splenocytes are isolated and resuspended to a concentration of 5×10⁶ cells/mL in media containing peptides representing either CD4+ or CD8+ T cell epitopes to HPV, PMA/ionomycin as a positive control, or DMSO as a mock stimulation.

### Intracellular Cytokine Staining.

Spleens are isolated according to the methods outlined for ELISpots, and 1×10⁶ cells are added to cells containing media in a total volume of 200 μL per well. Each well contains peptides representing either CD4+ or CD8+ T cell epitopes to HPV, PMA/ionomycin as a positive control, or DMSO as a mock stimulation. After 1 hour, GolgiPlug™ protein transport inhibitor (BD Biosciences) is added to each well. Cells are incubated for another 5 hours. Following incubation, cells are surface stained for CD8+(53-6.7), CD4+ (GK1.5), B220 (B238128), Gr-1 (RB6-8C5), CD16/32 (M93) using standard methods. Following surface staining, cells are fixed and stained for intracellular proteins as per standard methods for IFNγ (RPA-T8), IL-2 (JES6-5H4), and TNF (MP6-XT22). Cells are then subsequently analyzed on a flow cytometer and the acquired FCS files analyzed using FlowJo software version 10.4.1.

### Antibodies.

Antibody responses to measure total HPV E6/E7-specific IgG are measured using ELISA kits from Alpha Diagnostic International as per manufacturer's instructions.

### Example 7

### Evaluation of Modified Alphavirus Vectors with Lengthened Poly(A)

This Example describes the results of in vitro experiments performed to evaluate RNA replication activity of modified alphavirus srRNA constructs with varying lengths of poly(A).

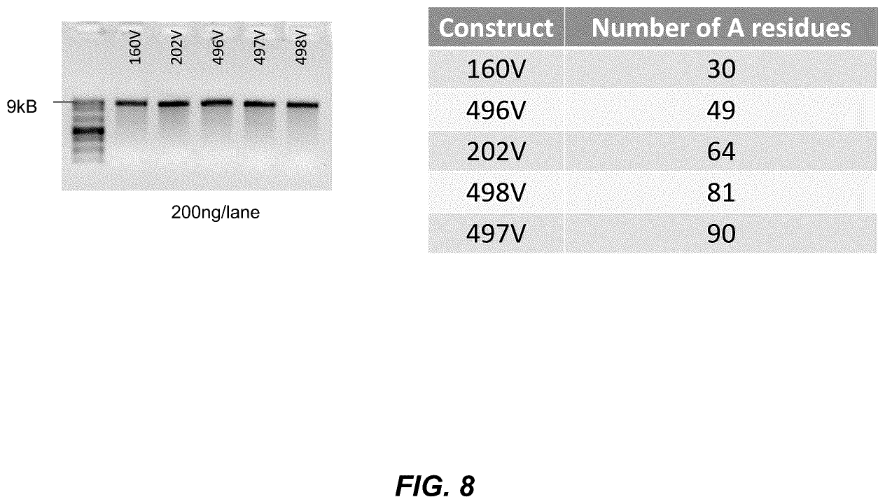

A VEE empty vector was linearized with SpeI and NotI (fragment 1), a PCR product containing the hemagglutinin (HA) gene from influenza (Genbank AY651334) was generated with 30 bp homology ends to fragment 1 and fragment 3 (fragment 2), and a synthetic DNA fragment (fragment 3) containing a poly(A) sequence with varying lengths (e.g., with 30, 49, 64, 81, or 90 adenylate residues), followed by a SapI site, a T7 terminator, and 30 bp homology ends to fragment 2 and to the linearized empty vector (fragment 1) were combined by three-fragment Gibson Assembly® procedure. The length of the poly(A) sequence in the resulting plasmids was verified by Sanger sequencing. RNA was then prepared by in vitro transcription using the plasmid DNA templates linearized by SapI enzymatic digestion as described in Example 5 above. RNA was purified by LiCl precipitation. Subsequently, RNA integrity was assessed by electrophoresis analysis on agarose gel, and the results are summarized in FIG. 8).

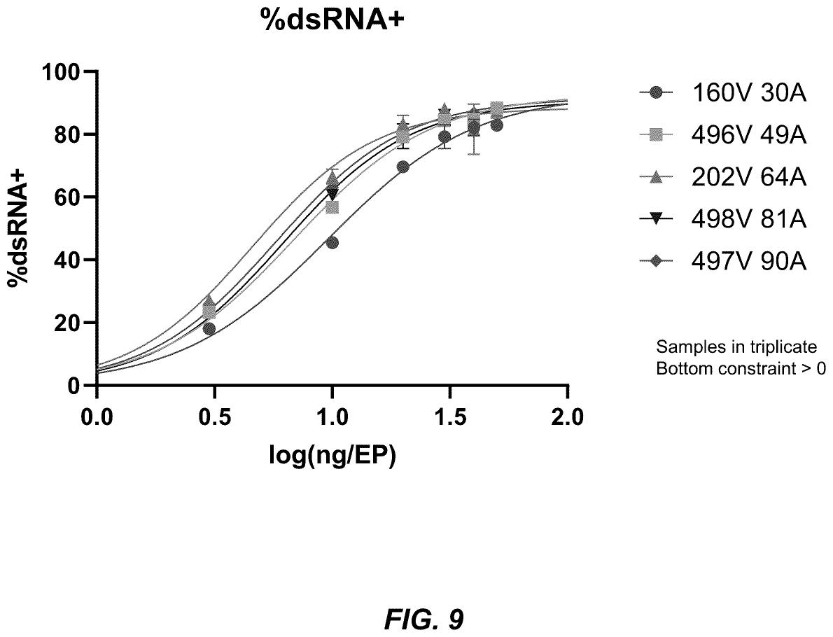

To quantify RNA replication activity, the srRNA constructs were transformed by electroporation into 8E5 BHK-21 cells (e.g. 4D-Nucleofector™, Lonza) for each sample. Each srRNA construct was transformed in triplicate at doses of 3, 10, 20, 30, 40, and 50 ng. At 20 h following transformation, the cells were fixed and permeabilized (eBioscience™ Foxp3/Transcription Factor Staining Buffer Set, Invitrogen) and stained using a PE-conjugated anti-dsRNA mouse monocolonal antibody (J2, Scicons) to quantify the frequency of dsRNA+ cells (cells in which RNA replication is detectable) by fluorescence flow cytometry. The frequency of dsRNA+ cells in each sample at each log-transformed RNA dose for each srRNA construct is shown in FIG. 9.

Using Prism (GraphPad Software), log(EC50) values were calculated for each srRNA construct by fitting the data to a 4PL curve with a bottom constraint >0. The log(EC50) values and the backtransformed EC50 values are shown in Table 1. The EC50 values represent the dose of RNA necessary for half-maximum RNA replication frequency.

TABLE 1
Summary of EC₅₀ (RNA dose for half-maximal activity) calculated
from fitting the data shown in FIG. 9 to a 4PL curve.

| srRNA | Log(EC50) | EC50 (ng RNA) |
|---|---|---|
| 160V 30A | 0.9809 | 9.570 |
| 496V 49A | 0.8366 | 6.865 |
| 202V 64A | 0.6616 | 4.588 |
| 498V 81A | 0.7908 | 6.177 |
| 497V 90A | 0.7610 | 5.768 |

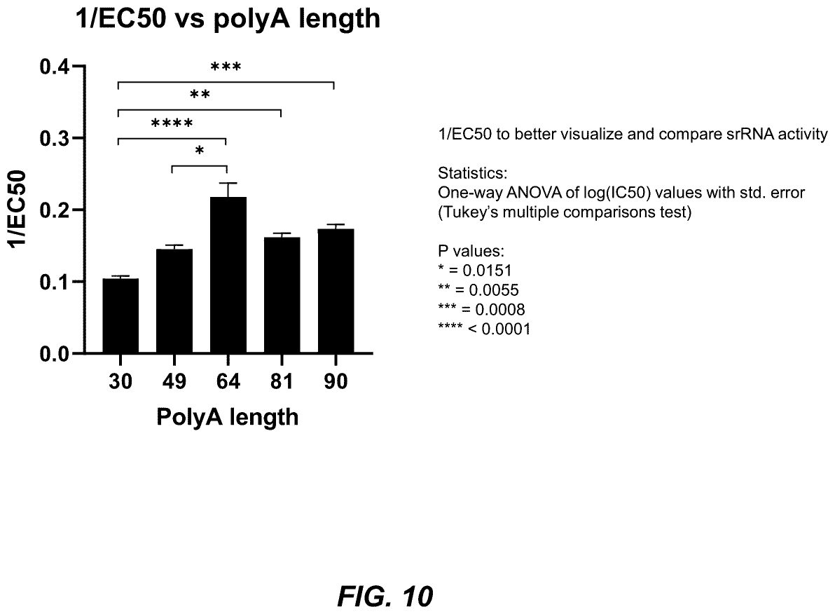

To better visualize the results, since the lowest EC50 value functionally equates to the highest replication activity per mass RNA, the inverse of EC50 is shown in FIG. 10. A one-way ANOVA statistical analysis was performed using Prism (GraphPad Software) to determine statistical significance between the experimental EC50 values and are illustrated in FIG. 10 and shown in Table 2. In these experiments, srRNA constructs with the shortest poly(A) tail consisting of 30 adenylate (A) residues were found to exhibit the lowest RNA replication activity. It was also found that srRNA constructs with the median length poly(A) consisting of 64 A residues exhibited the highest activity. As shown in FIG. 10, the order of activity was as follows: 30A<49A<81A<90A<64A.

All srRNA constructs with poly(A) lengths greater than 30A exhibited significantly higher activity than the reference srRNA construct containing a poly(A) sequence with 30 A residues. srRNA constructs with 64 A residues exhibited significantly higher activity than srRNA constructs with 49 A residues, but srRNA constructs with longer poly(A) sequences (e.g., 81A, 90A) did not exhibit significantly higher activity than 49A.

In these experiments, srRNA constructs with the longest poly(A) sequences tested (e.g., 81A, 90A) trended towards lower activity than srRNA constructs with the median 64A length, however the activity was not found to be significantly lower than the activity from 64A. These data suggests that a poly(A) of 64A or at least 64A results in significantly more activity for srRNA constructs.

TABLE 2
Results of a one-way ANOVA statistical test performed
to determine significant differences between the Log(EC₅₀)
values calculated from the data shown in FIG. 9.

| Tukey's multiple comparisons | | | Adjusted |
|---|---|---|---|
| test (One-way ANOVA) | Mean Diff. | Summary | P Value |
| 160V 30A vs. 496V 49A | 0.1443 | ns | 0.0619 |
| 160V 30A vs. 202V 64A | 0.3192 | **** | <0.0001 |
| 160V 30A vs. 498V 81A | 0.1901 | ** | 0.0055 |
| 160V 30A vs. 497V 90A | 0.2199 | *** | 0.0008 |
| 496V 49A vs. 202V 64A | 0.1749 | * | 0.0151 |
| 496V 49A vs. 498V 81A | 0.0458 | ns | 0.9108 |
| 496V 49A vs. 497V 90A | 0.0756 | ns | 0.618 |
| 202V 64A vs. 498V 81A | −0.1291 | ns | 0.1303 |
| 202V 64A vs. 497V 90A | −0.0993 | ns | 0.3616 |
| 498V 81A vs. 497V 90A | 0.0298 | ns | 0.9805 |

ns = not significant.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A nucleic acid construct comprising a modified alphavirus genome or replicon RNA, wherein a substantial portion of the nucleic acid sequence encoding the viral structural proteins of the modified alphavirus genome or replicon RNA is replaced by a synthetic adaptor molecule configured for facilitating insertion of a heterologous sequence into the modified alphavirus genome or replicon RNA, and wherein the synthetic adaptor molecule having the Formula I: 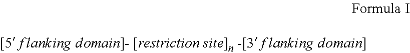
wherein
a) n is an integer from 1 to 6;
b) the restriction site is cleavable by a restriction endonuclease; and
c) the 5′ flanking domain and 3′ flanking domain each comprise a nucleic acid sequence predicted to have minimal secondary structure.

2. The nucleic acid construct of claim 1, wherein the sequences of the 5′ flanking domain has a folding ΔG value of the minimum free energy (MFE) structure higher than a predefined threshold value.

3. The nucleic acid construct of any one of claims 1 to 2, wherein the 5′ flanking domain does not comprise a sequence which encodes an RNA sequence capable of forming a stem-loop structure.

4. The nucleic acid construct of any one of claims 1 to 3, wherein the 5′ flanking domain comprises a coding sequence for an autoproteolytic peptide.

5. The nucleic acid construct of claim 4, wherein the autoproteolytic peptide comprises one or more autoproteolytic cleavage sequences derived from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

6. The nucleic acid construct of any one of claims 4 to 5, wherein the coding sequence for the autoproteolytic peptide is incorporated upstream of the restriction site(s).

7. The nucleic acid construct of any one of claims 1 to 6, wherein the 5′ flanking domain comprises an internal ribosomal entry site (IRES).

8. The nucleic acid construct of claim 7, wherein the IRES element is incorporated upstream of the restriction site(s).

9. The nucleic acid construct of any one of claims 1 to 8, wherein the 5′ flanking domain does not comprise a translation start site in any reading frame.

10. The nucleic acid construct of any one of claims 1 to 8, wherein the 5′ flanking domain comprises a translation start site or a part thereof as the last nucleotides of the 5′ adaptor sequence.

11. The nucleic acid construct of any one of claims 1 to 8, wherein the 5′ flanking domain comprises a methionine codon as the last three nucleotides of the 5′ adaptor sequence.

12. The nucleic acid construct of any one of claims 1 to 11, wherein the 5′ flanking domain has a length of from about 15 nucleotides to about 35 nucleotides.

13. The nucleic acid construct of claim 12, wherein the 5′ flanking domain has a length of about 30 nucleotides.

14. The nucleic acid construct of any one of claims 1 to 13, wherein the 5′ flanking domain comprises a nucleic acid sequence having at least 70%, at least 80% at least 90%, or at least 95% sequence identity to SEQ ID NO: 1.

15. The nucleic acid construct of any one of claims 1 to 14, wherein the sequences of the 3′ flanking domain has a folding ΔG value of the minimum free energy (MFE) structure higher than a predefined threshold value.

16. The nucleic acid construct of any one of claims 1 to 15, wherein the 5′ flanking domain does not comprise a sequence which encodes an RNA sequence capable of forming a stem-loop structure.

17. The nucleic acid construct of any one of claims 1 to 16, wherein the 3′ flanking domain comprise a translation stop codon as the first three nucleotides of the 3′ adaptor sequence.

18. The nucleic acid construct of claim 17, wherein the stop codon is selected from TAG, TAA, or TGA.

19. The nucleic acid construct of any one of claims 1 to 18, wherein the 3′ flanking domain comprises a nucleic acid sequence having at least 70%, at least 80% at least 90%, or at least 95% sequence identity to SEQ ID NO: 2.

20. The nucleic acid construct of any one of claims 1 to 19, wherein the synthetic adaptor molecule comprises a nucleic acid sequence having at least 70%, at least 80% at least 90%, or at least 95% sequence identity to SEQ ID NO: 20.

21. The nucleic acid construct of any one of claims 1 to 20, wherein the restriction site is cleavable by a restriction enzyme selected from Type I restriction enzymes, Type II restriction enzymes, Type III restriction enzymes, Type IV restriction enzymes, and Type V restriction enzymes.

22. The nucleic acid construct of claim 21, wherein the restriction site is cleavable by a Type II restriction enzyme.

23. The nucleic acid construct of claim 22, wherein the restriction site is cleavable by SpeI or an isoschizomer thereof.

24. A nucleic acid construct comprising a modified alphavirus genome or replicon RNA comprising a poly(A) tail, wherein the poly(A) tail does not comprise a 3′ non-A residue.

25. The nucleic acid construct of any one of claims 1 to 24, further comprising an additional restriction site engineered into the sequence encoding the poly(A) tail of the alphavirus genome or replicon RNA.

26. The nucleic acid construct of any one of claims 1 to 24, further comprising an additional restriction site incorporated at the end of the sequence encoding the poly(A) tail of the alphavirus genome or replicon RNA.

27. The nucleic acid construct of claim 26, wherein the additional restriction site is cleavable by a Type IIS restriction enzyme or a homing endonuclease.

28. The nucleic acid construct of claim 27, wherein the Type IIS restriction enzyme is AcuI, AlwI, Alw26I, BaeI, BbiI, BbsI, BbsI-HF, BbvI, BccI, BceAI, BcgI, BciVI, BcoDI, BfuAI, BmrI, BpmI, BpuEI, BsaI, BsaI-HF, BsaI-HFv2, BsaXI, BseGI, BseRI, BsgI, BsmAI, BsmBI-v2, BsmFI, BsmI, BspCNI, BspMI, BspQI, BsrDI, BsrI, BtgZI, BtsCI, BtsI-v2, BtsIMutI, CspCI, EarI, EciI, Eco31I, Esp3I, FauI, FokI, HgaI, HphI, HpyAV, LpuI, MboII, MlyI, Mmel, MnlI, NmeAIII, PaqCI, PleI, SapI, or SfaNI.

29. The nucleic acid construct of claim 27, wherein the homing endonuclease is I-CeuI, I-SceI, PI-PspI, or PI-SceI.

30. A nucleic acid construct comprising a modified alphavirus genome or replicon RNA comprising a poly(A) tail, wherein the lengthened sequence encoding the poly(A) tail is longer than 34 residues.

31. The nucleic acid construct of claim 30, wherein the lengthened poly(A) tail has a length ranging from about 30 to about 120 adenylate residues.

32. The nucleic acid construct of any one of claims 30 to 31, wherein the lengthened poly(A) tail has a length of about 30, about 40, about 50, about 60, about 70, about 80, about 90, and about 100 adenylate residues.

33. The nucleic acid construct of any one of claims 1 to 31, wherein the modified genome or replicon RNA is of a virus belonging to the Alphavirus genus of the Togaviridae family.

34. The nucleic acid construct of claim 33, wherein the modified genome or replicon RNA is of an alphavirus belonging to the VEEV/EEEV group, or the SFV group, or the SINV group.

35. The nucleic acid construct of claim 34, wherein the alphavirus is Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), or Buggy Creek virus.

36. The nucleic acid construct of claim 35, wherein the alphavirus is Venezuelan equine encephalitis virus (VEEV), Eastern Equine Encephalitis virus (EEEV), Chikungunya virus (CHIKV), or Sindbis virus (SINV).

37. The nucleic acid construct of any one of claims 1 to 36, further comprising one or more expression cassettes, wherein each of the expression cassettes comprises a promoter operably linked to a heterologous nucleic acid sequence.

38. The nucleic acid construct of claim 37, wherein at least one of the expression cassettes comprises a subgenomic (sg) promoter operably linked to a heterologous nucleic acid sequence.

39. The nucleic acid construct of claim 38, wherein the sg promoter is a 26S subgenomic promoter.

40. The nucleic acid construct of any one of claims 1 to 39, further comprising one or more untranslated regions (UTRs).

41. The nucleic acid construct of claim 40, wherein at least one of the UTRs is a heterologous UTR.

42. The nucleic acid construct of any one of claims 1 to 41, wherein the 5′ flanking domain does not encode for an RNA sequence capable of forming a stem-loop structure with a sequence located immediately upstream thereof (e.g., in the sgRNA 5′ UTR) or with a sequence located immediately downstream thereof (e.g., within the coding sequence of a GOI).

43. The nucleic acid construct of any one of claims 1 to 42, wherein the 3′ flanking domain does not encode for an RNA sequence capable of forming a stem-loop structure with a sequence located immediately upstream thereof (e.g., within the coding sequence of a GOI) or with a sequence located immediately downstream (e.g., in the 3′ UTR).

44. The nucleic acid construct of any one of claims 1 to 43, wherein the 5′ flanking domain and/or 3′ flanking domain does not comprise a sequence having complementarity with a sequence located within the 3′ UTR.

45. The nucleic acid construct of any one of claims 1 to 43, wherein the 5′ flanking domain and/or 3′ flanking domain does not comprise a sequence having complementarity with the 3′ end of the 3′ UTR.

46. The nucleic acid construct of any one of claims 37 to 45, wherein at least one of expression cassettes comprises a coding sequence for a gene of interest (GOI).

47. The nucleic acid construct of claim 46, wherein the GOI coding sequence comprises a stop codon positioned upstream of the 3′ flanking domain of the synthetic adaptor molecule.

48. The nucleic acid construct of any one of claims 46 to 47, wherein the GOI encodes a polypeptide selected from the group consisting of a therapeutic polypeptide, a prophylactic polypeptide, a diagnostic polypeptide, a nutraceutical polypeptide, an industrial enzyme, and a reporter polypeptide.

49. The nucleic acid construct of any one of claims 46 to 48, wherein the GOI encodes a polypeptide selected from the group consisting of an antibody, an antigen, an immune modulator, an enzyme, a signaling protein, and a cytokine.

50. The nucleic acid construct of any one of claims 46 to 49, wherein the coding sequence of the GOI is optimized for expression at a level higher than the expression level of a reference coding sequence.

51. The nucleic acid construct of any one of claims 1 to 50, wherein the nucleic acid construct is incorporated within a vector.

52. The nucleic acid construct of claim 51, wherein the vector is a self-replicating RNA (srRNA) vector.

53. The nucleic acid construct of any one of claims 1 to 52, wherein the nucleic acid sequence has at least 70%, at least 75%, at least 80%, at least 85%, at least 90%, at least 95%, at least 96%, at least 97%, at least 98%, at least 99%, or 100% sequence identity to a nucleic acid sequence selected from the group consisting of SEQ ID NOS: 3-27.

54. A recombinant cell comprising a nucleic acid construct according to any one of claims 1 to 53.

55. The recombinant cell of claim 54, wherein the recombinant cell is a eukaryotic cell.

56. The recombinant cell of claim 55, wherein the recombinant cell is an animal cell.

57. The recombinant cell of claim 56, wherein the animal cell is a vertebrate animal cell or an invertebrate animal cell.

58. The recombinant cell of claim 57, wherein the recombinant cell is a mammalian cell.

59. The recombinant cell of claim 58, wherein the recombinant cell is selected from the group consisting of African green monkey kidney cell (Vero cell), baby hamster kidney (BHK) cell, Chinese hamster ovary cell (CHO cell), human A549 cell, human cervix cell, human CHME5 cell, human epidermoid larynx cell, human fibroblast cell, human HEK-293 cell, human HeLa cell, human HepG2 cell, human HUH-7 cell, human MRC-5 cell, human muscle cell, mouse 3T3 cell, mouse connective tissue cell, mouse muscle cell, and rabbit kidney cell.

60. A cell culture comprising at least one recombinant cell according to any one of claims 54-59, and a culture medium.

61. A transgenic animal comprising a nucleic acid construct according to any one of claims 1 to 53.

62. The transgenic animal of claim 61, wherein the animal is a vertebrate animal or an invertebrate animal.

63. The transgenic animal of claim 62, wherein the animal is a mammalian.

64. The transgenic animal of claim 63, wherein the mammalian is a non-human mammalian.

65. A method for producing a recombinant RNA molecule, comprising (i) rearing a transgenic animal according to any one of claims 61-64, or (ii) culturing a recombinant cell according to any one of claims 54-59 under conditions such that the recombinant RNA molecule is produced.

66. The method of claim 65, wherein the transgenic animal or the recombinant cell comprising a nucleic acid construct according to any one of claims 24-53, and wherein the sequence encoding the recombinant RNA molecule is optionally digested by a restriction enzyme capable of cleaving the restriction site engineered after the end of the sequence encoding the poly(A) tail.

67. A recombinant RNA molecule produced by the method of any one of claims 65-66.

68. The recombinant RNA molecule of claim 67, wherein the recombinant RNA molecule exhibits enhanced biologic activity.

69. A method for producing a polypeptide of interest, comprising (i) rearing a transgenic animal comprising a nucleic acid construct according to any one of claims 48-53, or (ii) culturing a recombinant cell comprising a nucleic acid construct according to any one of claims 48-50 under conditions wherein the polypeptide encoded by the GOI is produced.

70. A method for producing a polypeptide of interest in a subject, comprising administering to the subject a nucleic acid construct according to any one of claims 48 to 53.

71. The method of claim 70, wherein the subject is a vertebrate animal or an invertebrate animal.

72. The method of claim 71, wherein the subject is a mammalian subject.

73. The method of claim 72, wherein the mammalian subject is a human subject.

74. A recombinant polypeptide produced by the method of any one of claims 69-73.

75. A pharmaceutical composition comprising a pharmaceutically acceptable excipient and:
a) a nucleic acid construct of any one of claims 1-53;
b) a recombinant RNA molecule of claim 67;
c) a recombinant cell of any one of claims 54-59; and/or
d) a recombinant polypeptide of claim 74.

76. The pharmaceutical composition of claim 75, comprising a nucleic acid construct of any one of claims 1-53, and a pharmaceutically acceptable excipient.

77. The pharmaceutical composition of claim 75, comprising a recombinant RNA molecule of claim 67, and a pharmaceutically acceptable excipient.

78. The pharmaceutical composition of claim 75, comprising a recombinant cell of any one of claims 54-59, and a pharmaceutically acceptable excipient.

79. The pharmaceutical composition of claim 75, comprising a recombinant polypeptide of claim 74, and a pharmaceutically acceptable excipient.

80. The pharmaceutical composition of any one of claims 75-79, wherein the composition is formulated in a liposome, a lipid-based nanoparticle (LNP), or a polymer nanoparticle.

81. The pharmaceutical composition of any one of claims 75-80, wherein the composition is an immunogenic composition.

82. The pharmaceutical composition of claim 81, wherein the immunogenic composition is formulated as a biotherapeutic.

83. The pharmaceutical composition of claim 81, wherein the immunogenic composition is formulated as a vaccine.

84. The pharmaceutical composition of any one of claims 75-80, wherein the composition is substantially non-immunogenic to a subject.

85. The pharmaceutical composition of claim 84, wherein the non-immunogenic composition is formulated as a biotherapeutic.

86. The pharmaceutical composition of claim 84, wherein the non-immunogenic composition is formulated as a vaccine.

87. The pharmaceutical composition of any one of claims 75-80, wherein the pharmaceutical composition is formulated as an adjuvant.

88. The pharmaceutical composition of any one of claims 75-87, wherein the pharmaceutical composition is formulated for one or more of intranasal administration, transdermal administration, intraperitoneal administration, intramuscular administration, intranodal administration, intratumoral administration, intraarticular administration, intravenous administration, subcutaneous administration, intravaginal, and oral administration.

89. A method for modulating an immune response in a subject in need thereof, the method comprises administering to the subject a composition comprising:
a) a nucleic acid construct of any one of claims 1-53;
b) a recombinant RNA molecule of claim 67;
c) a recombinant cell of any one of claims 54-59;
d) a recombinant polypeptide of claim 74; and/or
e) a pharmaceutical composition of any one of claims 75-88.

90. A method for preventing and/or treating a health condition in a subject in need thereof, the method comprises prophylactically or therapeutically administering to the subject a composition comprising:
a) a nucleic acid construct of any one of claims 1-53;
b) a recombinant RNA molecule of claim 67;
c) a recombinant cell of any one of claims 54-59;
d) a recombinant polypeptide of claim 74; and/or
e) a pharmaceutical composition of any one of claims 75-88.

91. The method of any one of claims 89-90, wherein the health condition is a proliferative disorder, inflammatory disorder, autoimmune disorder, or a microbial infection.

92. The method of any one of claims 89-91, wherein the subject has or is suspected of having a health condition associated with proliferative disorder, inflammatory disorder, autoimmune disorder, or a microbial infection.

93. The method of any one of claims 89-92, wherein the composition is administered to the subject individually as a single therapy (monotherapy) or as a first therapy in combination with at least one additional therapies.

94. The method of claim 93, wherein the at least one additional therapies is selected from the group consisting of chemotherapy, radiotherapy, immunotherapy, hormonal therapy, toxin therapy, targeted therapy, and surgery.

95. A kit for modulating an immune response, for the prevention, and/or for the treatment of a health condition or a microbial infection, the kit comprising:
a) a nucleic acid construct of any one of claims 1-53;
b) a recombinant RNA molecule of claim 67;
c) a recombinant cell of any one of claims 54-59;
d) a recombinant polypeptide of claim 74; and/or
e) a pharmaceutical composition of any one of claims 75-88.


SEQUENCE LISTING (31 sequences)

SEQ ID NO: 1 - DNA, 30 bp, Artificial sequence
  Synthetic construct
  MISC_FEATURE: Exemplary 5′ flanking domain
ctggagacgtggaggagaaccctggaccta

SEQ ID NO: 2 - DNA, 30 bp, Artificial sequence
  Synthetic construct
  MISC_FEATURE: Exemplary 3′ flanking domain
tgaccgctacgccccaatgacccgaccagc

SEQ ID NO: 3 - DNA, 7793 bp, Artificial sequence
  Synthetic construct
  MISC_FEATURE: DNA template sequence within a VEE empty vector with universal adaptors
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactggtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatctggagacgtggaggagaaccctggacctactagtgaccgctacgccccaatgacccgaccagctaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaa

SEQ ID NO: 4 - DNA, 8179 bp, Artificial sequence
  Synthetic construct
  MISC_FEATURE: DNA template sequence within a CHIKV S27 empty vector with universal adaptors
gatggctgcgtgagacacacgtagcctaccagtttcttactgctctactctgcaaagcaagagattaagaacccatcatggatcctgtgtacgtggacatagacgctgacagcgcctttttgaaggccctgcaacgtgcgtaccccatgtttgaggtggaacctaggcaggtcacaccgaatgaccatgctaatgctagagcgttctcgcatctagctataaaactaatagagcaggaaattgatcccgactcaaccatcctggatattggtagtgcgccagcaaggaggatgatgtcggacaggaagtaccactgcgtttgcccgatgcgcagtgcagaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagatgtatcatgtagacagagagcagacgtcgcgatataccaagacgtctatgctgtacacgcacccacgtcgctataccaccaggcgattaaaggggtccgattggcgtactgggtagggtttgacacaaccccgttcatgtacaatgccatggcgggtgcctacccctcatactcgacaaattgggcagatgagcaggtactgaaggctaagaacataggattatgttcaacagacctgacggaaggtagacgaggcaaattgtctattatgagaggaaaaaagctagaaccgtgcgaccgtgtgctgttctcagtagggtcaacgctctacccggaaagccgtaagctacttaagagctggcacctaccatcggtgttccatttaaagggcaagctcagcttcacatgccgctgtgatacagtggtttcgtgcgaaggctacgtcgttaagagaataacgatgagcccaggcctttacggaaaaaccacagggtatgcggtaacccaccacgcagacggattcctgatgtgcaagaccaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaactatatgattcccgtggtcgcccaagccttcagtaagtgggcaaaggagtgccggaaagacatggaagatgaaaaactcctgggggtcagagaaagaacactgacctgctgctgtctatgggcatttaagaagcagaaaacacacacggtctacaagaggcctgatacccagtcaattcagaaggttcaggccgagtttgacagctttgtggtaccgagcctgtggtcgtccgggttgtcaatcccgttgaggactagaatcaaatggttgttaagcaaggtgccaaaaaccgacctgaccccatacagcggggacgcccaagaagcccgggacgcagaaaaagaagcagaggaagaacgagaagcagaactgactcttgaagccctaccaccccttcaggcagcacaggaagatgttcaggtcgaaatcgacgtggaacagcttgaggacagagcgggtgcaggaataatagagactccgagaggagctatcaaagttactgcccaaccaacagaccacgtcgtgggagagtacttggttctttccccgcagaccgtactacgtagccaaaagcttagcctgattcacgctttggcggagcaagtgaagacgtgcacgcacagcggacgagcagggaggtatgcggtcgaagcgtacgacggcagagtcctagtgccctcaggctacgcaatctcgcctgaagacttccagagcctaagcgaaagcgcaacgatggtgtacaacgaaagagagttcgtaaacagaaagctacaccatattgcgatgcatggaccagccctgaacaccgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatatgaagggctaaaaatccgccctgcctgcccatacaaaattgcagtcataggagtcttcggagtaccaggatctggcaagtcagctattatcaagaacctagttaccaggcaagacctggtgactagcggaaagaaagaaaactgccaagaaatcaccaccgacgtgatgagacagagaggtctagagatatctgcacgtacggttgactcgctgctcttgaatggatgtaacagaccagtcgacgtgttgtacgtagacgaggcgtttgcgtgccactctggaacgttacttgcattgatcgccttggtgagaccaagacagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgactgccattgtgtcatcgttgcattacgaaggcaaaatgcgcactacgaatgagtacaacaagccgattgtagtggacactacaggctcaacaaaacctgaccctggagatctcgtgttaacgtgcttcagaggatgggttaaacaactgcaaattgactatcgtggacacgaggtcatgacagcagccgcatcccaagggttaaccagaaaaggagtttacgcagttaggcaaaaagttaacgaaaacccgctttatgcatcaacgtcagagcacgtcaacgtactcctaacgcgtacggaaggtaaactggtatggaagacactctccggtgacccgtggataaagacgctgcagaacccaccgaaaggaaacttcaaagcaactattaaggagtgggaggtggagcatgcatcaataatggcgggcatctgcagtcaccaaatgacctttgatacattccaaaacaaagccaacgtttgttgggctaagagtttggtccctatcctcgaaacagcggggataaaactaaacgacaggcagtggtcccagataattcaagccttcaaagaagacaaagcatattcacccgaagtagccctgaatgaaatatgcacgcgcatgtatggggtggatctagacagcgggctattttctaaaccgttggtgtctgtgtattacgcggataaccactgggataataggcctggagggaagatgttcggattcaaccccgaggcagcatccattctagaaagaaagtatccatttacaaaagggaagtggaacatcaacaagcagatctgcgtgactaccaggaggatagaagacttcaaccctaccaccaacattataccggccaacaggagactaccacactcattagtggccgaacaccgcccagtaaaaggggaaagaatggaatggctggttaacaagataaacggccaccacgtgctcctggtcagtggctgtagccttgcactgcctactaagagagtcacttgggtagcgccactaggtgtccgcggagcggactatacatacaacctagagttgggtctgccagcaacgcttggtaggtatgacctagtggtcataaacatccacacaccttttcgcatacaccattatcaacagtgcgtagaccacgcaatgaaactgcaaatgctcgggggtgactcattgagactgctcaaaccgggtggctctctattgatcagagcatatggttacgcagatagaaccagtgaacgagtcatctgcgtattgggacgcaagtttagatcatctagagcgttgaaaccaccatgtgtcaccagcaacactgagatgttttttctattcagcaactttgacaatggcagaaggaatttcacaactcatgtcatgaacaatcaactgaatgcagcctttgtaggacaggccacccgagcaggatgtgcaccgtcgtaccgggtaaaacgcatggatatcgcgaagaacgatgaagagtgcgtagtcaacgccgccaaccctcgcgggttaccaggtgacggtgtttgcaaggcagtatacaaaaaatggccggagtcctttaagaacagtgcaacaccagtgggaaccgcaaaaacagtcatgtgcggtacgtatccagtaatccacgccgttggaccaaacttctctaattattcggagtctgaaggggaccgagaattggcggctgcctatcgagaagtcgcaaaggaggtaactagactgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtggagctgctggatgagcacatctccatagactgcgatgttgttcgcgtgcaccctgacagcagcttggcaggcagaaaaggatacagcaccacggaaggcgcactgtactcatatctagaagggacccgttttcaccaaacggcagtggatatggcagagatatatactatgtggccaaagcaaacagaggccaacgagcaagtttgcctatatgccctgggggaaagtattgaatcgatcaggcagaaatgcccggtggatgatgcagatgcatcatctcccccgaaaactgtcccgtgcctctgccgttacgccatgacaccagaacgcgttacccgacttcgcatgaaccatgtcacaagcataattgtgtgttcttcgtttccccttccaaagtacaaaatagaaggagtgcaaaaagtcaaatgctccaaggtaatgctatttgaccacaacgtgccatcgcgcgtaagtccaagggaatacagaccttcccaggagtctgtacaggaagcgagtacgaccacgtcactgacgcatagccaattcgatctaagcgttgacggcaagatactgcccgtcccgtcagacctggatgctgacgccccagccctagaaccagcccttgacgacggggcgatacacacgttgccatctgcaaccggaaaccttgcggccgtgtctgactgggtaatgagcaccgtacctgtcgcgccgcccagaagaaggcgagggagaaacctgactgtgacatgcgacgagagagaagggaatataacacccatggctagcgtccgattctttagggcagagctgtgtccagtcgtacaagaaacagcggagacgcgtgacacagctatgtctcttcaggcaccgccgagtaccgccacggaactgagtcacccgccgatctccttcggtgcaccaagcgagacgttccccatcacatttggggacttcaacgaaggagaaatcgaaagcttgtcttctgagctactaactttcggagacttcctacccggagaagtggatgatttgacagatagcgactggtccacgtgctcagacacggacgacgagttacgactagacagggcaggtgggtatatattctcgtcggacactggtccaggtcatttacaacagaagtcagtacgccagtcagtgctgccggtgaacaccctggaggaagtccacgaggagaagtgttacccacctaagctggatgaagcaaaggagcaactactacttaagaaactccaggagagtgcatccatggccaacagaagcaggtatcagtcgcgcaaagtagaaaacatgaaagcaacaatcatccagagactaaagagaggctgtagattatacttaatgtcagagaccccaaaagtccctacctaccggaccacatatccggcgcctgtgtactcgcctccgattaacgtccgactgtccaaccccgagtccgcagtggcagcatgcaatgagttcttggctagaaactatccaactgtttcatcataccaaatcaccgacgagtatgatgcatatctagacatggtggacgggtcggagagttgtctggaccgagcgacattcaatccgtcaaaacttaggagctacccaaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctatcaggataacaactgagaatttaacaacctatgttactaaactaaaggggccaaaagcagcagcgctatttgcaaaaacccataatctgctgccactgcaggaagtgccaatggataggttcacagtagacatgaaaagggatgtgaaggtgactcctggtacaaagcacacagaggaaagacctaaggtacaggttatacaggcggctgaacccttggcaacagcatacctatgtgggattcacagagagctggttaggaggctgaacgccgtcctcctacccaatgtacatacactatttgacatgtctgccgaggatttcgatgccatcatagccgcacactttaagccaggagacactgttttagaaacggacatagcctcctttgataagagccaagatgattcacttgcgcttactgctttaatgctgttagaggatttaggggtggatcactccctgttggacttgatagaggctgctttcggagagatttccagctgtcatctaccgacaggtacgcgcttcaagttcggcgccatgatgaaatctggtatgttcctaactctgttcgtcaacacactgctaaatatcaccatcgccagccgagtgctggaagatcgtctgacaaaatccgcgtgcgcagccttcatcggcgacgacaacataatacatggagtcgtctccgatgaattgatggcagccagatgcgccacttggatgaacatggaagtgaagatcatagatgcagttgtatcccagaaagccccttacttttgtggagggtttatactgcacgatatcgtgacaggaacagcttgcagagtggcagacccgctaaaaaggctatttaaactgggcaaaccgctagcggcaggtgacgaacaagatgaggatagaagacgagcgctggctgacgaagtggtcagatggcaacgaacagggctaattgatgagttggagaaagcggtatactctaggtatgaagtgcagggtatatcagttgtggtaatgtccatggccacctttgcaagctccagatccaacttcgagaagctcagaggacccgtcgtaactttgtacggcggtcctaaataggtacgcactacagctacctattttgcagaagccgacagtaagtacctaaacactaatcagctacactggagacgtggaggagaaccctggacctactagtgaccgctacgccccaatgacccgaccagcttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttcaaaaaaaaaaaaaaaaaaaaaaaaaaaaaaaaaaaa

SEQ ID NO: 5 - DNA, 8179 bp, Artificial sequence
  Synthetic construct
  MISC_FEATURE: DNA template sequence within a CHIKV DRDE empty vector with universal adaptors
gatggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgctaattatgcgagaaagctagcatctgccgcaggaaaagtcctggacagaaacatctctggaaagatcggggacttacaagcagtaatggccgtgccagacaaggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgctgtgcaagactaccgacacggttgacggcgaaagagtgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatatgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcgacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgatcgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgaccttgtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtgacggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgactgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttatgactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaagttcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtgaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtcataactttgtacggcggtcctaaataggtacgcactacagctacctattttgcagaagccgacagcaagtatctaaacactaatcagctacactggagacgtggaggagaaccctggacctactagtgaccgctacgccccaatgacccgaccagcttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttcaaaaaaaaaaaaaaaaaaaaaaaaaaaaaaaaaaaa

SEQ ID NO: 6 - DNA, 8065 bp, Artificial sequence
  Synthetic construct
  MISC_FEATURE: DNA template sequence within a EEEV FL93-939 empty vector with universal adaptors
gatagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctactaagctcattgagggagaagtggatacagaccaggtgatcctggatattgggagcgcgcctgtaaggcacacgcattccaaacataagtaccactgcatttgcccaatgaagagcgcagaagaccctgacagactctaccgctatgcagacaagcttagaaaaagtgatgtcactgacaaatgtattgcctctaaggccgcggacctgctaacagtaatgtcgacgcctgacactgagacaccctcgttatgcatgcacactgactcaacttgccggtaccacggctccgtggccgtatatcaggatgtatatgcagtgcatgcaccgacttccatttactaccaggcgctgaaaggtgtacgaactatctattggatcgggtttgatactacaccgttcatgtacaagaacatggcaggcgcctaccctacatacaacacaaattgggccgatgaaagtgtgttggaagccagaaatatagggctgggtagttcagacttgcacgaaaagagtttcggaaaagtatccattatgaggaagaagaaattacaacccactaataaagtaatattttctgtggggtcaactatttatactgaagagagaatactgttacgcagttggcatctacctaatgtctttcatctaaaaggtaaaactagctttacaggcagatgtaacaccatcgtcagctgcgaaggttacgttgtcaagaagattacgctcagtcctgggatttacgggaaagtggataatcttgcttcgaccatgcaccgagagggattcttaagttgcaaggttacagacacgttaagaggggagagggtctctttccccgtatgtacgtacgtgccagcgacactgtgcgaccagatgaccgggatactggcgactgacgtcagtgtcgatgacgcccagaagctgctggttgggctcaaccagcgaattgtcgtcaatggcagaacacaacgtaacacaaataccatgcagaattatctattaccagtggtcgcccaggcgttctcgcggtgggcgcgggaacaccgcgcagacctggaggacgaaaaagggctaggggtacgggaacgttccctagtcatgggctgctgctgggctttcaaaactcacaagatcacatccatttacaagagacctgggactcaaactatcaagaaggtgcccgccgtattcaattccttcgtcatcccacaaccaaccagctatgggcttgatataggattgcgtcgccgaattaagatgctattcgacgcaaagaaggcacccgctccaattattactgaggccgacgtcgcacaccttaaaggcctgcaggatgaagctgaagccgtggctgaggctgaagccgtgcgtgcagcactacctccacttctgccggaggtcgataaggagaccgtagaggccgatatcgacctgatcatgcaggaggcaggagcaggcagcgtggagacacctagacgacacatcaaggtcacgacgtatccaggagaagaaatgatcggctcgtacgcagtgctctcaccacaagcggtccttaacagcgagaagctagcttgcattcacccgttagctgagcaagtgctcgtgatgactcacaaagggcgcgcaggacgatacaaggtagagccataccacggtagagttatcgtccctagtggtacagctataccaatccccgatttccaggctctgagtgaaagtgcaaccatagtatttaacgaacgggagttcgttaaccgttacttacaccacattgccgttaacggaggggcattgaatacagatgaagagtactacaaggttgtgaaaagcactgagacagactctgagtacgtatttgacatcgacgcaaagaagtgcgtgaagaaaggggatgccggaccaatgtgcctggtcggcgagttagtagacccgccattccacgaatttgcgtacgagagtttaaaaacacgtcctgctgcaccacacaaagtgcctactatcggagtctatggagtcccaggttccggaaagtctggtataatcaaaagcgctgttaccaagcgtgatctggtggtcagtgcaaagaaagaaaattgcatggaaatcattaaagacgtcaaacgtatgcgcggcatggacatcgccgcccgcacagtggattcggtgctgctaaatggggtaaaacactccgtcgacacactgtacatagacgaggcattcgcttgccatgcagggaccctgctagcacttatcgccatcgtcaagccaaagaaagttgtattgtgtggagatccgaaacaatgcggcttctttaacatgatgtgtctaaaagtacattttaaccacgagatatgcacagaagtgtatcacaagagtatttctcggcgatgcactaagacagtgacatccattgtttctaccctgttctatgataaacggatgagaactgtcaacccatgcaatgataagatcataatagataccaccagtactaccaaacctttaaaggatgacataatattaacctgctttagagggtgggttaagcaactgcagattgactacaagaaccacgagatcatgaccgcagcggcctcacaggggcttactagaaaaggggtatacgcagtgcgctacaaggtcaatgagaacccactatacgcacagacatctgagcatgtgaatgtattacttacacgcacagaaaaacgtatagtatggaagactttggccggtgacccttggatcaagacgttgacagcatcgtatccgggtaatttcaccgccacactggaagaatggcaagctgagcatgacgctatcatggcgaaaatacttgagacaccagctagcagcgacgttttccaaaataaagtgaacatctgctgggccaaagcgctagaacctgtgttggccaccgccaatattacgctgacccgctcgcagtgggagactattccagcgttcaaggatgacaaagcgtattcgcctgagatggccttaaactttttctgcaccagattctttggtgtcgacatcgacagcgggttgttctccgcgccaactgttccgctgacttacaccaatgaacactgggataatagcccaggtccaaacatgtatgggttgtgcatgcgcactgctaaagaacttgcacgtcggtatccttgtattctgaaagccgtggatacaggtagagtggctgacgttcgcacagacactatcaaagactataacccgctaataaatgtggtaccccttaatagaagactcccacactcgttggttgtcacacacagatacactgggaacggtgattactcccagctagtgactaagatgaccggaaaaaccgtactcgtagtgggtacacctatgaacataccaggaaagagagttgagacattaggcccaagcccacaatgtacatataaagcggaattggacctgggcattcctgccgctttaggcaaatatgacatcatctttattaacgtgaggactccctaccgacaccaccactaccaacagtgcgaggaccatgcgatccaccacagcatgcttaccagaaaagcagtggaccatttgaacaaaggcggtacgtgcatcgcattgggctatgggactgcggacagagccaccgagaacattatctctgcagtcgcccgctcattcaggttctcacgtgtgtgccagccgaagtgtgcctgggaaaacactgaggtcgcgttcgtgtttttcggcaaggacaacggcaaccatctccaagatcaagataggctgagtgttgtgttaaacaacatataccaagggtcaactcaacatgaagctggcagagcacctgcgtatagagtggtgcgcggcgacataacaaagagcaatgatgaggttattgttaacgcggcgaacaacaaagggcaacctggtggcggtgtgtgtggcgccctttacaggaagtggcctggagcttttgacaagcagccggtagcaactggtaaagcgcacctcgtcaagcattctccgaacgtcatccatgccgttggccctaatttttctaggctatcagaaaacgaaggagaccagaaattgtctgaagtgtacatggacattgccagaattatcaacaacgagaggtttactaaagtctccattccgttgttatctaccggcatttacgcaggtggtaaggacagggttatgcaatcgctgaaccatttattcacagccatggatactaccgacgcagacatcaccatttactgtctagataagcaatgggagtcaagaataaaggaagctatcacccggaaggaaagtgttgaagagcttactgaggatgacagaccagttgacattgaactggtacgggtgcacccgttgagcagcttggcaggtagacctggttattcaaccaccgagggcaaggtgtattcgtacctagaggggactaggtttcaccaaactgccaaagacatagctgaaatttacgctatgtggcctaacaagcaagaagcaaacgagcagatttgcttatatgtgttgggagagagtatgaacagcatccgctctaagtgtccagttgaagagtcggaggcctcttccccccctcacaccatcccgtgtctgtgcaactatgcaatgactgcagagcgagtttacagattacgtatggcgaagaatgaacaattcgcagtttgttcgtcctttcagttaccgaaatacaggattacaggggttcagaaaattcaatgcagtaaacctgtgatattctccggcactgtaccaccggccatacatccaagaaaattcgcatctgtgacagtggaagacactccggtggtccaacctgaaaggttggtgcctaggcgacctgcaccgcctgtgcccgtacctgcaagaatccccagccctccatgtacatcgaccaacggatcgacgaccagtatacaatcactgggggaggatcaaagcgcatctgcttctagcggagctgaaatctctgtagaccaggtttcgctatggagcatacccagcgctactgggttcgatgtgcgtacctcctcatcgttgagtctagagcagtctacctttccgacaatggttgtcgaagcagagattcacgccagtcaaggatcactgtggagtatacccagtatcaccggatctgaaacccgcgttccgtcacctccaagtcagggtagcagacattccaccccatctgtaagtgcttcacacacgtccgtggacttaatcacgtttgacagcgttgcagagattttggaagatttcagtcgttcgccgtttcaatttttgtctgaaatcaaacctatccctgcacctcgtacccgagttaataacatgagccgcagcgcagacacgatcaaaccaattccaaagccgcgtaaatgccaggtgaagtacacgcagccacctggcgtcgccagggccatatcggcagcggaatttgacgagtttgtgcggaggcactcgaattgacggtacgaagcgggcgcgtacattttctcatccgagacaggacaagggcacctgcaacaaaaatccacgcggcaatgcaaactccagtatccaatcctggagcgttccgtccatgagaaattttacgccccgcgcctcgatctcgagcgtgagaagctgttgcagaagaaactacaattgtgtgcttctgaaggtaatcggagcaggtatcagtctcgtaaagtagagaacatgaaggcaatcaccgttgagcgtctactgcaggggataggctcatatctctctgcagaaccgcaaccagttgaatgctacaaagtcacctatcctgctcccatgtattcaagtactgcaagcaacagcttttcatcagcagaagtggccgtcaaagtctgcaacctagtactgcaagagaattttcccaccgtagccagctataacataacggatgagtatgatgcctatcttgacatggtggacggagcatcctgctgtttagatactgccactttttgcccagctaaattgaggagctttccaaagaagcacagttatttgcggcctgagatacgatcagcagtgccatcaccgattcaaaacacgctccagaatgtactagcagcagccacgaaacggaattgcaatgtcactcaaatgagggaacttccagtgttggattcagctgccttcaacgtggagtgtttcaaaaagtacgcctgtaacgatgagtactgggacttctacaagacaaacccgataagactcaccgcagaaaatgttactcagtatgttactaagttaaagggacccaaagcagctgccctttttgcgaaaacgcataacttacagccattgcatgagataccaatggatagattcgtgatggaccttaaacgggatgtcaaggtcacacccgggacaaaacatactgaagaaagaccaaaagttcaggtgatacaggcagctgatccacttgcaaccgcctacctatgtggtatacatcgagagcttgtgcgcaggttgaacgcagtgctgctaccgaatatccacactttgtttgacatgtctgcagaagattttgatgctatcattgccgaacactttcaattcggcgacgcggtgttagagacagacatagcttcttttgataaaagcgaggacgatgctatcgccatgtccgctctaatgattcttgaagacctaggagttgatcaggcactgttaaacctaattgaggcagcctttgggaacataacatctgtgcacttaccaacaggcacccgatttaagttcggggcaatgatgaaatctgggatgtttttgacactctttatcaataccgttgtcaatatcatgatcgctagccgcgtgctccgcgagcggctgaccacttccccctgcgcagcatttatcggcgacgacaacatcgtgaaaggggttacatctgacgcgctgatggcagagcggtgcgccacgtggttgaacatggaagtgaagatcatcgatgcagtagtcggagtaaaggcaccgtacttttgcggagggttcatcgtagtcgatcagattacaggaactgcgtgcagagtcgccgaccccctgaagagactgtttaagctaggtaagccgcttccactggacgatgaccaagacgtcgacaggcgcagagctctgcatgatgaagcggcacgttggaacagaattggcatcaccgaagagctggtgaaagcagttgaatcacgctacgaggtgaactacgtgtcactaatcatcacagcgttgaccacattagcatcttcagttagcaactttaaacacataagaggtcaccccataaccctctacggctgacctaaataggttgtgcattagtacctaacctatttatattatattgctatctaaatatcagagctggagacgtggaggagaaccctggacctactagtgaccgctacgccccaatgacccgaccagctaacatcttgtcaaccacataacactacaggcagtgtataaggctgtcttactaaacactaaattcaccctagttcgatgtacttccgagctatggtgacggtggtgcataatgccgccgatgcagtgcataaggctgctatattaccaaattataacactaagggcagtgcataatgctgctcctaagtaattttatacacactttataatcaggcataattgccgtatatacaattacactacaggtaatataccgcctcttataaacactacaggcagcgcataatgctgtcttttatatcaatttacaaaatcatattaattttttcttttatgtttttattttgtttttaatatttcaaaaaaaaaaaaaaaaaaaaaaaaaaaaaaaaaaaaaaaaaaaa

SEQ ID NO: 7 - DNA, 9764 bp, Artificial sequence
  Synthetic construct
  MISC_FEATURE: DNA template sequence within a EEEV FL93-939 vector containing a GOI (universal adaptors)
gatagggtacggtgtagaggcaaccaccctatttccacctatccaaaatggagaaagttcatgttgacttagacgcagacagcccattcgtcaagtcactgcaaagatgctttccacattttgagatagaagcaacgcaggtcactgacaatgaccatgctaatgctagggcgttttcgcacctagctactaagctcattgagggagaagtggatacagaccaggtgatcctggatattgggagcgcgcctgtaaggcacacgcattccaaacataagtaccactgcatttgcccaatgaagagcgcagaagaccctgacagactctaccgctatgcagacaagcttagaaaaagtgatgtcactgacaaatgtattgcctctaaggccgcggacctgctaacagtaatgtcgacgcctgacactgagacaccctcgttatgcatgcacactgactcaacttgccggtaccacggctccgtggccgtatatcaggatgtatatgcagtgcatgcaccgacttccatttactaccaggcgctgaaaggtgtacgaactatctattggatcgggtttgatactacaccgttcatgtacaagaacatggcaggcgcctaccctacatacaacacaaattgggccgatgaaagtgtgttggaagccagaaatatagggctgggtagttcagacttgcacgaaaagagtttcggaaaagtatccattatgaggaagaagaaattacaacccactaataaagtaatattttctgtggggtcaactatttatactgaagagagaatactgttacgcagttggcatctacctaatgtctttcatctaaaaggtaaaactagctttacaggcagatgtaacaccatcgtcagctgcgaaggttacgttgtcaagaagattacgctcagtcctgggatttacgggaaagtggataatcttgcttcgaccatgcaccgagagggattcttaagttgcaaggttacagacacgttaagaggggagagggtctctttccccgtatgtacgtacgtgccagcgacactgtgcgaccagatgaccgggatactggcgactgacgtcagtgtcgatgacgcccagaagctgctggttgggctcaaccagcgaattgtcgtcaatggcagaacacaacgtaacacaaataccatgcagaattatctattaccagtggtcgcccaggcgttctcgcggtgggcgcgggaacaccgcgcagacctggaggacgaaaaagggctaggggtacgggaacgttccctagtcatgggctgctgctgggctttcaaaactcacaagatcacatccatttacaagagacctgggactcaaactatcaagaaggtgcccgccgtattcaattccttcgtcatcccacaaccaaccagctatgggcttgatataggattgcgtcgccgaattaagatgctattcgacgcaaagaaggcacccgctccaattattactgaggccgacgtcgcacaccttaaaggcctgcaggatgaagctgaagccgtggctgaggctgaagccgtgcgtgcagcactacctccacttctgccggaggtcgataaggagaccgtagaggccgatatcgacctgatcatgcaggaggcaggagcaggcagcgtggagacacctagacgacacatcaaggtcacgacgtatccaggagaagaaatgatcggctcgtacgcagtgctctcaccacaagcggtccttaacagcgagaagctagcttgcattcacccgttagctgagcaagtgctcgtgatgactcacaaagggcgcgcaggacgatacaaggtagagccataccacggtagagttatcgtccctagtggtacagctataccaatccccgatttccaggctctgagtgaaagtgcaaccatagtatttaacgaacgggagttcgttaaccgttacttacaccacattgccgttaacggaggggcattgaatacagatgaagagtactacaaggttgtgaaaagcactgagacagactctgagtacgtatttgacatcgacgcaaagaagtgcgtgaagaaaggggatgccggaccaatgtgcctggtcggcgagttagtagacccgccattccacgaatttgcgtacgagagtttaaaaacacgtcctgctgcaccacacaaagtgcctactatcggagtctatggagtcccaggttccggaaagtctggtataatcaaaagcgctgttaccaagcgtgatctggtggtcagtgcaaagaaagaaaattgcatggaaatcattaaagacgtcaaacgtatgcgcggcatggacatcgccgcccgcacagtggattcggtgctgctaaatggggtaaaacactccgtcgacacactgtacatagacgaggcattcgcttgccatgcagggaccctgctagcacttatcgccatcgtcaagccaaagaaagttgtattgtgtggagatccgaaacaatgcggcttctttaacatgatgtgtctaaaagtacattttaaccacgagatatgcacagaagtgtatcacaagagtatttctcggcgatgcactaagacagtgacatccattgtttctaccctgttctatgataaacggatgagaactgtcaacccatgcaatgataagatcataatagataccaccagtactaccaaacctttaaaggatgacataatattaacctgctttagagggtgggttaagcaactgcagattgactacaagaaccacgagatcatgaccgcagcggcctcacaggggcttactagaaaaggggtatacgcagtgcgctacaaggtcaatgagaacccactatacgcacagacatctgagcatgtgaatgtattacttacacgcacagaaaaacgtatagtatggaagactttggccggtgacccttggatcaagacgttgacagcatcgtatccgggtaatttcaccgccacactggaagaatggcaagctgagcatgacgctatcatggcgaaaatacttgagacaccagctagcagcgacgttttccaaaataaagtgaacatctgctgggccaaagcgctagaacctgtgttggccaccgccaatattacgctgacccgctcgcagtgggagactattccagcgttcaaggatgacaaagcgtattcgcctgagatggccttaaactttttctgcaccagattctttggtgtcgacatcgacagcgggttgttctccgcgccaactgttccgctgacttacaccaatgaacactgggataatagcccaggtccaaacatgtatgggttgtgcatgcgcactgctaaagaacttgcacgtcggtatccttgtattctgaaagccgtggatacaggtagagtggctgacgttcgcacagacactatcaaagactataacccgctaataaatgtggtaccccttaatagaagactcccacactcgttggttgtcacacacagatacactgggaacggtgattactcccagctagtgactaagatgaccggaaaaaccgtactcgtagtgggtacacctatgaacataccaggaaagagagttgagacattaggcccaagcccacaatgtacatataaagcggaattggacctgggcattcctgccgctttaggcaaatatgacatcatctttattaacgtgaggactccctaccgacaccaccactaccaacagtgcgaggaccatgcgatccaccacagcatgcttaccagaaaagcagtggaccatttgaacaaaggcggtacgtgcatcgcattgggctatgggactgcggacagagccaccgagaacattatctctgcagtcgcccgctcattcaggttctcacgtgtgtgccagccgaagtgtgcctgggaaaacactgaggtcgcgttcgtgtttttcggcaaggacaacggcaaccatctccaagatcaagataggctgagtgttgtgttaaacaacatataccaagggtcaactcaacatgaagctggcagagcacctgcgtatagagtggtgcgcggcgacataacaaagagcaatgatgaggttattgttaacgcggcgaacaacaaagggcaacctggtggcggtgtgtgtggcgccctttacaggaagtggcctggagcttttgacaagcagccggtagcaactggtaaagcgcacctcgtcaagcattctccgaacgtcatccatgccgttggccctaatttttctaggctatcagaaaacgaaggagaccagaaattgtctgaagtgtacatggacattgccagaattatcaacaacgagaggtttactaaagtctccattccgttgttatctaccggcatttacgcaggtggtaaggacagggttatgcaatcgctgaaccatttattcacagccatggatactaccgacgcagacatcaccatttactgtctagataagcaatgggagtcaagaataaaggaagctatcacccggaaggaaagtgttgaagagcttactgaggatgacagaccagttgacattgaactggtacgggtgcacccgttgagcagcttggcaggtagacctggttattcaaccaccgagggcaaggtgtattcgtacctagaggggactaggtttcaccaaactgccaaagacatagctgaaatttacgctatgtggcctaacaagcaagaagcaaacgagcagatttgcttatatgtgttgggagagagtatgaacagcatccgctctaagtgtccagttgaagagtcggaggcctcttccccccctcacaccatcccgtgtctgtgcaactatgcaatgactgcagagcgagtttacagattacgtatggcgaagaatgaacaattcgcagtttgttcgtcctttcagttaccgaaatacaggattacaggggttcagaaaattcaatgcagtaaacctgtgatattctccggcactgtaccaccggccatacatccaagaaaattcgcatctgtgacagtggaagacactccggtggtccaacctgaaaggttggtgcctaggcgacctgcaccgcctgtgcccgtacctgcaagaatccccagccctccatgtacatcgaccaacggatcgacgaccagtatacaatcactgggggaggatcaaagcgcatctgcttctagcggagctgaaatctctgtagaccaggtttcgctatggagcatacccagcgctactgggttcgatgtgcgtacctcctcatcgttgagtctagagcagtctacctttccgacaatggttgtcgaagcagagattcacgccagtcaaggatcactgtggagtatacccagtatcaccggatctgaaacccgcgttccgtcacctccaagtcagggtagcagacattccaccccatctgtaagtgcttcacacacgtccgtggacttaatcacgtttgacagcgttgcagagattttggaagatttcagtcgttcgccgtttcaatttttgtctgaaatcaaacctatccctgcacctcgtacccgagttaataacatgagccgcagcgcagacacgatcaaaccaattccaaagccgcgtaaatgccaggtgaagtacacgcagccacctggcgtcgccagggccatatcggcagcggaatttgacgagtttgtgcggaggcactcgaattgacggtacgaagcgggcgcgtacattttctcatccgagacaggacaagggcacctgcaacaaaaatccacgcggcaatgcaaactccagtatccaatcctggagcgttccgtccatgagaaattttacgccccgcgcctcgatctcgagcgtgagaagctgttgcagaagaaactacaattgtgtgcttctgaaggtaatcggagcaggtatcagtctcgtaaagtagagaacatgaaggcaatcaccgttgagcgtctactgcaggggataggctcatatctctctgcagaaccgcaaccagttgaatgctacaaagtcacctatcctgctcccatgtattcaagtactgcaagcaacagcttttcatcagcagaagtggccgtcaaagtctgcaacctagtactgcaagagaattttcccaccgtagccagctataacataacggatgagtatgatgcctatcttgacatggtggacggagcatcctgctgtttagatactgccactttttgcccagctaaattgaggagctttccaaagaagcacagttatttgcggcctgagatacgatcagcagtgccatcaccgattcaaaacacgctccagaatgtactagcagcagccacgaaacggaattgcaatgtcactcaaatgagggaacttccagtgttggattcagctgccttcaacgtggagtgtttcaaaaagtacgcctgtaacgatgagtactgggacttctacaagacaaacccgataagactcaccgcagaaaatgttactcagtatgttactaagttaaagggacccaaagcagctgccctttttgcgaaaacgcataacttacagccattgcatgagataccaatggatagattcgtgatggaccttaaacgggatgtcaaggtcacacccgggacaaaacatactgaagaaagaccaaaagttcaggtgatacaggcagctgatccacttgcaaccgcctacctatgtggtatacatcgagagcttgtgcgcaggttgaacgcagtgctgctaccgaatatccacactttgtttgacatgtctgcagaagattttgatgctatcattgccgaacactttcaattcggcgacgcggtgttagagacagacatagcttcttttgataaaagcgaggacgatgctatcgccatgtccgctctaatgattcttgaagacctaggagttgatcaggcactgttaaacctaattgaggcagcctttgggaacataacatctgtgcacttaccaacaggcacccgatttaagttcggggcaatgatgaaatctgggatgtttttgacactctttatcaataccgttgtcaatatcatgatcgctagccgcgtgctccgcgagcggctgaccacttccccctgcgcagcatttatcggcgacgacaacatcgtgaaaggggttacatctgacgcgctgatggcagagcggtgcgccacgtggttgaacatggaagtgaagatcatcgatgcagtagtcggagtaaaggcaccgtacttttgcggagggttcatcgtagtcgatcagattacaggaactgcgtgcagagtcgccgaccccctgaagagactgtttaagctaggtaagccgcttccactggacgatgaccaagacgtcgacaggcgcagagctctgcatgatgaagcggcacgttggaacagaattggcatcaccgaagagctggtgaaagcagttgaatcacgctacgaggtgaactacgtgtcactaatcatcacagcgttgaccacattagcatcttcagttagcaactttaaacacataagaggtcaccccataaccctctacggctgacctaaataggttgtgcattagtacctaacctatttatattatattgctatctaaatatcagagctggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagctaacatcttgtcaaccacataacactacaggcagtgtataaggctgtcttactaaacactaaattcaccctagttcgatgtacttccgagctatggtgacggtggtgcataatgccgccgatgcagtgcataaggctgctatattaccaaattataacactaagggcagtgcataatgctgctcctaagtaattttatacacactttataatcaggcataattgccgtatatacaattacactacaggtaatataccgcctcttataaacactacaggcagcgcataatgctgtcttttatatcaatttacaaaatcatattaattttttcttttatgtttttattttgtttttaatatttcaaaaaaaaaaaaaaaaaaaaaaaaaaaaaaaaaaaaaaaaaaaa

SEQ ID NO: 8 - DNA, 9831 bp, Artificial sequence
  Synthetic construct
  MISC_FEATURE: DNA template sequence for a srRNA within a Sindbis AR86-Girdwood chimera 1 vector containing a GOI (universal adaptors)
gattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctatggagaaaatcgtcctcctgtttgctatagtgtcccttgtgaagagcgatcagatctgcatagggtatcatgccaataattccaccgaacaagtggacactattatggaaaagaatgtcacagttacacatgctcaggatatcttggaaaaaaaacacaacggaaagctctgcgatctcgatggtgtaaaaccacttatcctgcgggactgctctgttgcagggtggctgcttggaaaccccatgtgtgacgaatttatcaacgtccccgaatggtcttacatagttgaaaaagcaaatcctgtcaatgacctgtgctaccccggagactttaacgattatgaagagctgaagcatcttcttagtcgaatcaaccattttgagaagatccagattatcccaaagagctcttggagctcacatgaagcaagcctcggggtatcatctgcctgcccctatcaagggaagtctagtttcttcagaaacgtcgtgtggctcatcaaaaagaattcaacttaccctaccatcaagcgaagttataataatacaaatcaagaagatctgctggtgttgtggggcatacatcatcccaatgacgccgccgaacaaacaaagctctaccaaaatcccaccacttacatttctgtggggacatccactctcaaccaaaggctcgtgccacgcatcgctactcggagtaaagtcaatggacagtctgggcgaatggagtttttttggacaattctgaaacctaatgacgccataaacttcgagagcaacgggaacttcatcgcaccagagtatgcctataaaattgtgaaaaaaggcgattccacaattatgaagtctgaattggaatacggaaattgcaataccaaatgccagacaccaatgggtgccataaactcctctatgccctttcataacattcacccacttacaattggcgaatgtccaaaatatgtaaaatcaaatcgcttggttttggctacaggtttgcggaattctcctcaacgagaaagacgccgaaagaagagaggactgttcggtgcaatcgccggtttcatagagggaggttggcaaggaatggtagacggctggtacggttatcaccattcaaatgaacaaggttctgggtatgctgctgataaagaaagcacacaaaaggcaatcgatggcgtaaccaataaagtgaacagcataatcgacaagatgaatacccaatttgaagccgtagggagggaatttaataatctcgaacggcggatcgagaatttgaataagaaaatggaagatggatttttggacgtatggacatataatgccgaattgttggtccttatggagaacgaaagaacactggactttcatgactccaacgtcaagaatttgtatgacaaggtgcgcctccaactccgggataacgctaaagaactcggaaatggttgtttcgagttttaccacaagtgcgataatgagtgcatggaaagcgtgcgaaacggtacttacgattatccccaatattcagaggaagcccgactgaaacgggaggaaatcagtggcgtgaaattggaaagcatcggcatttatcagattcttagcatctatagtactgtcgcatcctctctggccctggctattatggttgctggcctctcactctggatgtgctctaacgggtccctccagtgccggatttgcatatgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaa

SEQ ID NO: 9 - DNA, 9622 bp, Artificial sequence
  Synthetic construct
  MISC_FEATURE: DNA template sequence for a srRNA construct within a Sindbis AR86-Girdwood chimera 2 vector containing aGOI (universal adaptors)
cgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctatggagaaaatcgtcctcctgtttgctatagtgtcccttgtgaagagcgatcagatctgcatagggtatcatgccaataattccaccgaacaagtggacactattatggaaaagaatgtcacagttacacatgctcaggatatcttggaaaaaaaacacaacggaaagctctgcgatctcgatggtgtaaaaccacttatcctgcgggactgctctgttgcagggtggctgcttggaaaccccatgtgtgacgaatttatcaacgtccccgaatggtcttacatagttgaaaaagcaaatcctgtcaatgacctgtgctaccccggagactttaacgattatgaagagctgaagcatcttcttagtcgaatcaaccattttgagaagatccagattatcccaaagagctcttggagctcacatgaagcaagcctcggggtatcatctgcctgcccctatcaagggaagtctagtttcttcagaaacgtcgtgtggctcatcaaaaagaattcaacttaccctaccatcaagcgaagttataataatacaaatcaagaagatctgctggtgttgtggggcatacatcatcccaatgacgccgccgaacaaacaaagctctaccaaaatcccaccacttacatttctgtggggacatccactctcaaccaaaggctcgtgccacgcatcgctactcggagtaaagtcaatggacagtctgggcgaatggagtttttttggacaattctgaaacctaatgacgccataaacttcgagagcaacgggaacttcatcgcaccagagtatgcctataaaattgtgaaaaaaggcgattccacaattatgaagtctgaattggaatacggaaattgcaataccaaatgccagacaccaatgggtgccataaactcctctatgccctttcataacattcacccacttacaattggcgaatgtccaaaatatgtaaaatcaaatcgcttggttttggctacaggtttgcggaattctcctcaacgagaaagacgccgaaagaagagaggactgttcggtgcaatcgccggtttcatagagggaggttggcaaggaatggtagacggctggtacggttatcaccattcaaatgaacaaggttctgggtatgctgctgataaagaaagcacacaaaaggcaatcgatggcgtaaccaataaagtgaacagcataatcgacaagatgaatacccaatttgaagccgtagggagggaatttaataatctcgaacggcggatcgagaatttgaataagaaaatggaagatggatttttggacgtatggacatataatgccgaattgttggtccttatggagaacgaaagaacactggactttcatgactccaacgtcaagaatttgtatgacaaggtgcgcctccaactccgggataacgctaaagaactcggaaatggttgtttcgagttttaccacaagtgcgataatgagtgcatggaaagcgtgcgaaacggtacttacgattatccccaatattcagaggaagcccgactgaaacgggaggaaatcagtggcgtgaaattggaaagcatcggcatttatcagattcttagcatctatagtactgtcgcatcctctctggccctggctattatggttgctggcctctcactctggatgtgctctaacgggtccctccagtgccggatttgcatatgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaa

SEQ ID NO: 10 - DNA, 9832 bp, Artificial sequence
  Synthetic construct
  MISC_FEATURE: DNA template sequence for a srRNA construct within a Sindbis AR86-Girdwood chimera 3 vector containing aGOI (universal adaptors)
gattggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaaa

SEQ ID NO: 11 - DNA, 9886 bp, Artificial sequence
  Synthetic construct
  MISC_FEATURE: DNA template sequence for a srRNA within a Sindbis AR86-Girdwood chimera 4 vector containing aGOI (universal adaptors)
gattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaaa

SEQ ID NO: 12 - DNA, 9241 bp, Artificial sequence
  Synthetic construct
  MISC_FEATURE: Partial sequence of VEE srRNA plasmid, containing GOI flanked by universal adaptors
ccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactggtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatctggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagctaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaa

SEQ ID NO: 13 - DNA, 9878 bp, Artificial sequence
  Synthetic construct
  MISC_FEATURE: Partial sequence of the plasmid encoding the CHIKV S27 srRNA construct, containing GOI flanked byuniversal adaptors
gatggctgcgtgagacacacgtagcctaccagtttcttactgctctactctgcaaagcaagagattaagaacccatcatggatcctgtgtacgtggacatagacgctgacagcgcctttttgaaggccctgcaacgtgcgtaccccatgtttgaggtggaacctaggcaggtcacaccgaatgaccatgctaatgctagagcgttctcgcatctagctataaaactaatagagcaggaaattgatcccgactcaaccatcctggatattggtagtgcgccagcaaggaggatgatgtcggacaggaagtaccactgcgtttgcccgatgcgcagtgcagaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagatgtatcatgtagacagagagcagacgtcgcgatataccaagacgtctatgctgtacacgcacccacgtcgctataccaccaggcgattaaaggggtccgattggcgtactgggtagggtttgacacaaccccgttcatgtacaatgccatggcgggtgcctacccctcatactcgacaaattgggcagatgagcaggtactgaaggctaagaacataggattatgttcaacagacctgacggaaggtagacgaggcaaattgtctattatgagaggaaaaaagctagaaccgtgcgaccgtgtgctgttctcagtagggtcaacgctctacccggaaagccgtaagctacttaagagctggcacctaccatcggtgttccatttaaagggcaagctcagcttcacatgccgctgtgatacagtggtttcgtgcgaaggctacgtcgttaagagaataacgatgagcccaggcctttacggaaaaaccacagggtatgcggtaacccaccacgcagacggattcctgatgtgcaagaccaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaactatatgattcccgtggtcgcccaagccttcagtaagtgggcaaaggagtgccggaaagacatggaagatgaaaaactcctgggggtcagagaaagaacactgacctgctgctgtctatgggcatttaagaagcagaaaacacacacggtctacaagaggcctgatacccagtcaattcagaaggttcaggccgagtttgacagctttgtggtaccgagcctgtggtcgtccgggttgtcaatcccgttgaggactagaatcaaatggttgttaagcaaggtgccaaaaaccgacctgaccccatacagcggggacgcccaagaagcccgggacgcagaaaaagaagcagaggaagaacgagaagcagaactgactcttgaagccctaccaccccttcaggcagcacaggaagatgttcaggtcgaaatcgacgtggaacagcttgaggacagagcgggtgcaggaataatagagactccgagaggagctatcaaagttactgcccaaccaacagaccacgtcgtgggagagtacttggttctttccccgcagaccgtactacgtagccaaaagcttagcctgattcacgctttggcggagcaagtgaagacgtgcacgcacagcggacgagcagggaggtatgcggtcgaagcgtacgacggcagagtcctagtgccctcaggctacgcaatctcgcctgaagacttccagagcctaagcgaaagcgcaacgatggtgtacaacgaaagagagttcgtaaacagaaagctacaccatattgcgatgcatggaccagccctgaacaccgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatatgaagggctaaaaatccgccctgcctgcccatacaaaattgcagtcataggagtcttcggagtaccaggatctggcaagtcagctattatcaagaacctagttaccaggcaagacctggtgactagcggaaagaaagaaaactgccaagaaatcaccaccgacgtgatgagacagagaggtctagagatatctgcacgtacggttgactcgctgctcttgaatggatgtaacagaccagtcgacgtgttgtacgtagacgaggcgtttgcgtgccactctggaacgttacttgcattgatcgccttggtgagaccaagacagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgactgccattgtgtcatcgttgcattacgaaggcaaaatgcgcactacgaatgagtacaacaagccgattgtagtggacactacaggctcaacaaaacctgaccctggagatctcgtgttaacgtgcttcagaggatgggttaaacaactgcaaattgactatcgtggacacgaggtcatgacagcagccgcatcccaagggttaaccagaaaaggagtttacgcagttaggcaaaaagttaacgaaaacccgctttatgcatcaacgtcagagcacgtcaacgtactcctaacgcgtacggaaggtaaactggtatggaagacactctccggtgacccgtggataaagacgctgcagaacccaccgaaaggaaacttcaaagcaactattaaggagtgggaggtggagcatgcatcaataatggcgggcatctgcagtcaccaaatgacctttgatacattccaaaacaaagccaacgtttgttgggctaagagtttggtccctatcctcgaaacagcggggataaaactaaacgacaggcagtggtcccagataattcaagccttcaaagaagacaaagcatattcacccgaagtagccctgaatgaaatatgcacgcgcatgtatggggtggatctagacagcgggctattttctaaaccgttggtgtctgtgtattacgcggataaccactgggataataggcctggagggaagatgttcggattcaaccccgaggcagcatccattctagaaagaaagtatccatttacaaaagggaagtggaacatcaacaagcagatctgcgtgactaccaggaggatagaagacttcaaccctaccaccaacattataccggccaacaggagactaccacactcattagtggccgaacaccgcccagtaaaaggggaaagaatggaatggctggttaacaagataaacggccaccacgtgctcctggtcagtggctgtagccttgcactgcctactaagagagtcacttgggtagcgccactaggtgtccgcggagcggactatacatacaacctagagttgggtctgccagcaacgcttggtaggtatgacctagtggtcataaacatccacacaccttttcgcatacaccattatcaacagtgcgtagaccacgcaatgaaactgcaaatgctcgggggtgactcattgagactgctcaaaccgggtggctctctattgatcagagcatatggttacgcagatagaaccagtgaacgagtcatctgcgtattgggacgcaagtttagatcatctagagcgttgaaaccaccatgtgtcaccagcaacactgagatgttttttctattcagcaactttgacaatggcagaaggaatttcacaactcatgtcatgaacaatcaactgaatgcagcctttgtaggacaggccacccgagcaggatgtgcaccgtcgtaccgggtaaaacgcatggatatcgcgaagaacgatgaagagtgcgtagtcaacgccgccaaccctcgcgggttaccaggtgacggtgtttgcaaggcagtatacaaaaaatggccggagtcctttaagaacagtgcaacaccagtgggaaccgcaaaaacagtcatgtgcggtacgtatccagtaatccacgccgttggaccaaacttctctaattattcggagtctgaaggggaccgagaattggcggctgcctatcgagaagtcgcaaaggaggtaactagactgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtggagctgctggatgagcacatctccatagactgcgatgttgttcgcgtgcaccctgacagcagcttggcaggcagaaaaggatacagcaccacggaaggcgcactgtactcatatctagaagggacccgttttcaccaaacggcagtggatatggcagagatatatactatgtggccaaagcaaacagaggccaacgagcaagtttgcctatatgccctgggggaaagtattgaatcgatcaggcagaaatgcccggtggatgatgcagatgcatcatctcccccgaaaactgtcccgtgcctctgccgttacgccatgacaccagaacgcgttacccgacttcgcatgaaccatgtcacaagcataattgtgtgttcttcgtttccccttccaaagtacaaaatagaaggagtgcaaaaagtcaaatgctccaaggtaatgctatttgaccacaacgtgccatcgcgcgtaagtccaagggaatacagaccttcccaggagtctgtacaggaagcgagtacgaccacgtcactgacgcatagccaattcgatctaagcgttgacggcaagatactgcccgtcccgtcagacctggatgctgacgccccagccctagaaccagcccttgacgacggggcgatacacacgttgccatctgcaaccggaaaccttgcggccgtgtctgactgggtaatgagcaccgtacctgtcgcgccgcccagaagaaggcgagggagaaacctgactgtgacatgcgacgagagagaagggaatataacacccatggctagcgtccgattctttagggcagagctgtgtccagtcgtacaagaaacagcggagacgcgtgacacagctatgtctcttcaggcaccgccgagtaccgccacggaactgagtcacccgccgatctccttcggtgcaccaagcgagacgttccccatcacatttggggacttcaacgaaggagaaatcgaaagcttgtcttctgagctactaactttcggagacttcctacccggagaagtggatgatttgacagatagcgactggtccacgtgctcagacacggacgacgagttacgactagacagggcaggtgggtatatattctcgtcggacactggtccaggtcatttacaacagaagtcagtacgccagtcagtgctgccggtgaacaccctggaggaagtccacgaggagaagtgttacccacctaagctggatgaagcaaaggagcaactactacttaagaaactccaggagagtgcatccatggccaacagaagcaggtatcagtcgcgcaaagtagaaaacatgaaagcaacaatcatccagagactaaagagaggctgtagattatacttaatgtcagagaccccaaaagtccctacctaccggaccacatatccggcgcctgtgtactcgcctccgattaacgtccgactgtccaaccccgagtccgcagtggcagcatgcaatgagttcttggctagaaactatccaactgtttcatcataccaaatcaccgacgagtatgatgcatatctagacatggtggacgggtcggagagttgtctggaccgagcgacattcaatccgtcaaaacttaggagctacccaaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctatcaggataacaactgagaatttaacaacctatgttactaaactaaaggggccaaaagcagcagcgctatttgcaaaaacccataatctgctgccactgcaggaagtgccaatggataggttcacagtagacatgaaaagggatgtgaaggtgactcctggtacaaagcacacagaggaaagacctaaggtacaggttatacaggcggctgaacccttggcaacagcatacctatgtgggattcacagagagctggttaggaggctgaacgccgtcctcctacccaatgtacatacactatttgacatgtctgccgaggatttcgatgccatcatagccgcacactttaagccaggagacactgttttagaaacggacatagcctcctttgataagagccaagatgattcacttgcgcttactgctttaatgctgttagaggatttaggggtggatcactccctgttggacttgatagaggctgctttcggagagatttccagctgtcatctaccgacaggtacgcgcttcaagttcggcgccatgatgaaatctggtatgttcctaactctgttcgtcaacacactgctaaatatcaccatcgccagccgagtgctggaagatcgtctgacaaaatccgcgtgcgcagccttcatcggcgacgacaacataatacatggagtcgtctccgatgaattgatggcagccagatgcgccacttggatgaacatggaagtgaagatcatagatgcagttgtatcccagaaagccccttacttttgtggagggtttatactgcacgatatcgtgacaggaacagcttgcagagtggcagacccgctaaaaaggctatttaaactgggcaaaccgctagcggcaggtgacgaacaagatgaggatagaagacgagcgctggctgacgaagtggtcagatggcaacgaacagggctaattgatgagttggagaaagcggtatactctaggtatgaagtgcagggtatatcagttgtggtaatgtccatggccacctttgcaagctccagatccaacttcgagaagctcagaggacccgtcgtaactttgtacggcggtcctaaataggtacgcactacagctacctattttgcagaagccgacagtaagtacctaaacactaatcagctacactggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagcttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttcaaaaaaaaaaaaaaaaaaaaaaaaaaaaaaaaaaaa

SEQ ID NO: 14 - DNA, 9878 bp, Artificial sequence
  Synthetic construct
  MISC_FEATURE: Partial sequence of the plasmid encoding the CHIKV DRDE srRNA construct, containing GOI flanked byuniversal adaptor
gatggctgcgtgagacacacgtagcctaccagtttcttactgctctactctgcaaagcaagagattaataacccatcatggatcctgtgtacgtggacatagacgctgacagcgcctttttgaaggccctgcaacgtgcgtaccccatgtttgaggtggaaccaaggcaggtcacaccgaatgaccatgctaatgctagagcgttctcgcatctagctataaaactaatagagcaggaaattgaccccgactcaaccatcctggatatcggcagtgcgccagcaaggaggatgatgtcggacaggaagtaccactgcgtctgcccgatgcgcagtgcggaagatcccgagagactcgctaattatgcgagaaagctagcatctgccgcaggaaaagtcctggacagaaacatctctggaaagatcggggacttacaagcagtaatggccgtgccagacaaggagacgccaacattctgcttacacacagacgtctcatgtagacagagagcagacgtcgctatataccaagacgtctatgctgtacacgcacccacgtcgctataccaccaggcgattaaaggggtccgagtggcgtactgggttgggttcgacacaaccccgttcatgtacaatgccatggcgggtgcctacccctcatactcgacaaactgggcagatgagcaggtactgaaggctaagaacataggattatgttcaacagacctgacggaaggtagacgaggcaagttgtctattatgagagggaaaaagctaaaaccgtgcgaccgtgtgctgttctcagtagggtcaacgctctacccggaaagccgcaagctacttaagagctggcacctgccatcggtgttccatttaaagggcaaactcagcttcacatgccgctgtgatacagtggtttcgtgtgagggctacgtcgttaagagaataacgatgagcccaggcctttatggaaaaaccacagggtatgcggtaacccaccacgcagacggattcctgctgtgcaagactaccgacacggttgacggcgaaagagtgtcattctcggtgtgcacatacgtgccggcgaccatttgtgatcaaatgaccggcatccttgctacagaagtcacgccggaggatgcacagaagctgttggtggggctgaaccagagaatagtggttaacggcagaacgcaacggaatatgaacaccatgaaaaattatctgcttcccgtggtcgcccaagccttcagtaagtgggcaaaggagtgccggaaagacatggaagatgaaaaactcctgggggtcagagaaagaacactgacctgctgctgtctatgggcattcaagaagcagaaaacacacacggtctacaagaggcctgatacccagtcaattcagaaggttcaggccgagtttgacagctttgtggtaccgagtctgtggtcgtccgggttgtcaatccctttgaggactagaatcaaatggttgttaagcaaggtgccaaaaaccgacctgatcccatacagcggagacgcccgagaagcccgggacgcagaaaaagaagcagaggaagaacgagaagcagaactgactcgcgaagccctaccacctctacaggcagcacaggaagatgttcaggtcgaaatcgacgtggaacagcttgaggacagagcgggcgcaggaataatagagactccgagaggagctatcaaagttactgcccaaccaacagaccacgtcgtgggagagtacctggtactctccccgcagaccgtactacgtagccagaagctcagtctgattcacgctttggcggagcaagtgaagacgtgcacgcacaacggacgagcagggaggtatgcggtcgaagcgtacgacggccgagtcctagtgccctcaggctatgcaatctcgcctgaagacttccagagtctaagcgaaagcgcgacgatggtgtataacgaaagagagttcgtaaacagaaagctacaccatattgcgatgcacggaccagccctgaacaccgacgaagagtcgtatgagctggtgagggcagagaggacagaacacgagtacgtctacgacgtggatcagagaagatgctgtaagaaggaagaagccgcaggactggtactggtgggcgacttgactaatccgccctaccacgaattcgcatatgaagggctaaaaatccgccctgcctgcccatacaaaattgcagtcataggagtcttcggagtaccgggatctggcaagtcagctattatcaagaacctagttaccaggcaggacctggtgactagcggaaagaaagaaaactgccaagaaatcaccaccgacgtgatgagacagagaggtctagagatatctgcacgtacggttgactcgctgctcttgaatggatgcaacagaccagtcgacgtgttgtacgtagacgaggcgtttgcgtgccactctggaacgctacttgctttgatcgccttggtgagaccaaggcagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgaccgccattgtgtcatcgttgcattacgaaggcaaaatgcgcactacgaatgagtacaacaagccgatcgtagtggacactacaggctcaacaaaacctgaccctggagacctcgtgttaacgtgcttcagagggtgggttaaacaactgcaaattgactatcgtggatacgaggtcatgacagcagccgcatcccaagggttaaccagaaaaggagtttacgcagttagacaaaaagttaatgaaaacccgctctatgcatcaacgtcagagcacgtcaacgtactcctaacgcgtacggaaggtaaactggtatggaagacactttccggcgacccgtggataaagacgctgcagaacccaccgaaaggaaacttcaaagcaactattaaggagtgggaggtggagcatgcatcaataatggcgggcatctgcagtcaccaaatgaccttcgatacattccaaaataaagccaacgtttgttgggctaagagcttggtccctatcctcgaaacagcggggataaaactaaatgataggcagtggtctcagataattcaagccttcaaagaagacaaagcatactcacctgaagtagccctgaatgaaatatgtacgcgcatgtatggggtggatctagacagcgggctattttctaaaccgttggtgtctgtgtattacgcggataaccactgggataataggcctggagggaaaatgttcggatttaaccccgaggcagcatccattctagaaagaaagtatccattcacaaaagggaagtggaacatcaacaagcagatctgcgtgactaccaggaggatagaagactttaaccctaccaccaacatcataccggccaacaggagactaccacactcattagtggccgaacaccgcccagtaaaaggggaaagaatggaatggctggttaacaagataaacggccaccacgtgctcctggtcagtggctataaccttgcactgcctactaagagagtcacttgggtagcgccgttaggtgtccgcggagcggactacacatacaacctagagttgggtctgccagcaacgcttggtaggtatgaccttgtggtcataaacatccacacaccttttcgcatacaccattaccaacagtgcgtcgaccacgcaatgaaactgcaaatgctcgggggtgactcattgagactgctcaaaccgggcggctctctattgatcagagcatatggttacgcagatagaaccagtgaacgagtcatctgcgtattgggacgcaagtttagatcgtctagagcgttgaaaccaccatgtgtcaccagcaacactgagatgtttttcctattcagcaactttgacaatggcagaaggaatttcacaactcatgtcatgaacaatcaactgaatgcagccttcgtaggacaggtcacccgagcaggatgtgcaccgtcgtaccgggtaaaacgcatggacatcgcgaagaacgatgaagagtgcgtagtcaacgccgctaaccctcgcgggttaccgggtgacggtgtttgcaaggcagtatacaaaaaatggccggagtcctttaagaacagtgcaacaccagtgggaaccgcaaaaacagttatgtgcggtacgtatccagtaatccacgctgttggaccaaacttctctaattattcggagtctgaaggggaccgggaattggcagctgcctatcgagaagtcgcaaaggaagtaactaggctgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtagagctgctggatgagcacatctccatagactgcgatattgttcgcgtgcaccctgacagcagcttggcaggcagaaaaggatacagcaccacggaaggcgcactgtactcatatctagaagggacccgttttcatcagacggctgtggatatggcggagatacatactatgtggccaaagcaaacagaggccaatgagcaagtctgcctatatgccctgggggaaagtattgaatcgatcaggcagaaatgcccggtggatgatgcagacgcatcatctccccccaaaactgtcccgtgcctttgccgttacgctatgactccagaacgcgtcacccggcttcgcatgaaccacgtcacaagcataattgtgtgttcttcgtttcccctcccaaagtacaaaatagaaggagtgcaaaaagtcaaatgctctaaggtaatgctatttgaccacaacgtgccatcgcgcgtaagtccaagggaatatagatcttcccaggagtctgcacaggaggcgagtacaatcacgtcactgacgcatagtcaattcgacctaagcgttgatggcgagatactgcccgtcccgtcagacctggatgctgacgccccagccctagaaccagcactagacgacggggcgacacacacgctgccatccacaaccggaaaccttgcggccgtgtctgactgggtaatgagcaccgtacctgtcgcgccgcccagaagaaggcgagggagaaacctgactgtgacatgtgacgagagagaagggaatataacacccatggctagcgtccgattctttagggcagagctgtgtccggtcgtacaagaaacagcggagacgcgtgacacagcaatgtctcttcaggcaccaccgagtaccgccacggaaccgaatcatccgccgatctccttcggagcatcaagcgagacgttccccattacatttggggacttcaacgaaggagaaatcgaaagcttgtcttctgagctactaactttcggagacttcttaccaggagaagtggatgacttgacagacagcgactggtccacgtgctcagacacggacgacgagttatgactagacagggcaggtgggtatatattctcgtcggacaccggtccaggtcatttacaacagaagtcagtacgccagtcagtgctgccggtgaacaccctggaggaagtccacgaggagaagtgttacccacctaagctggatgaagcaaaggagcaactattacttaagaaactccaggagagtgcatccatggccaacagaagcaggtatcagtcgcgcaaagtagaaaacatgaaagcagcaatcatccagagactaaagagaggctgtagactatacttaatgtcagagaccccaaaagtccctacttaccggactacatatccggcgcctgtgtactcgcctccgatcaacgtccgattgtccaatcccgagtccgcagtggcagcatgcaatgagttcttagctagaaactatccaactgtctcatcataccaaattaccgacgagtatgatgcatatctagacatggtggacgggtcggagagttgcctggaccgagcgacattcaatccgtcaaaactcaggagctacccgaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaagttcgcatgcaaccaagaatactgggaagaatttgctgccagccctattaggataacaactgagaatttagcaacctatgttactaaactaaaagggccaaaagcagcagcgctattcgcaaaaacccataatctactgccactacaggaagtaccaatggataggttcacagtagatatgaaaagggacgtgaaggtgactcctggtacaaagcatacagaggaaagacctaaggtgcaggttatacaggcggctgaacccttggcgacagcatacctatgtgggattcacagagagctggttaggaggctgaacgccgtcctcctacccaatgtacatacactatttgacatgtctgccgaggatttcgatgccatcatagccgcacactttaagccaggagacactgttttggaaacggacatagcctcctttgataagagccaagatgattcacttgcgcttactgctttgatgctgttagaggatttaggggtggatcactccctgctggacttgatagaggctgctttcggagagatttccagctgtcacctaccgacaggtacgcgcttcaagttcggcgccatgatgaaatcaggtatgttcctaactctgttcgtcaacacattgttaaacatcaccatcgccagccgagtgctggaagatcgtctgacaaaatccgcgtgcgcggccttcatcggcgacgacaacataatacatggagtcgtctccgatgaattgatggcagccagatgtgccacttggatgaacatggaagtgaagatcatagatgcagttgtatccttgaaagccccttacttttgtggagggtttatactgcacgatactgtgacaggaacagcttgcagagtggcagacccgctaaaaaggctttttaaactgggcaaaccgctagcggcaggtgacgaacaagatgaagatagaagacgagcgctggctgacgaagtgatcagatggcaacgaacagggctaattgatgagctggagaaagcggtatactctaggtacgaagtgcagggtatatcagttgtggtaatgtccatggccacctttgcaagctccagatccaacttcgagaagctcagaggacccgtcataactttgtacggcggtcctaaataggtacgcactacagctacctattttgcagaagccgacagcaagtatctaaacactaatcagctacactggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagcttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttcaaaaaaaaaaaaaaaaaaaaaaaaaaaaaaaaaaaa

SEQ ID NO: 15 - DNA, 9886 bp, Artificial sequence
  Synthetic construct
  MISC_FEATURE: Partial sequence of the plasmid encoding the SINV Girdwood srRNA construct, containing GOI flanked byuniversal adaptors
gattggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaaaa

SEQ ID NO: 16 - DNA, 76 bp, Artificial sequence
  Synthetic construct
  MISC_FEATURE: DNA sequence encoding the CHIKV DRDE 5′ UTR
atggctgcgtgagacacacgtagcctaccagtttcttactgctctactctgcaaagcaagagattaataacccatc

SEQ ID NO: 17 - DNA, 513 bp, Artificial sequence
  Synthetic construct
  MISC_FEATURE: DNA sequence encoding the CHIKV S27 3′ UTR
cttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttc

SEQ ID NO: 18 - DNA, 18 bp, Artificial sequence
  Synthetic construct
  MISC_FEATURE: bacteriophage T7 RNA polymerase promoter
taatacgactcactatag

SEQ ID NO: 19 - DNA, 29 bp, Artificial sequence
  Synthetic construct
  MISC_FEATURE: T7 terminator sequence
aacccctctctaaacggaggggttttttt

SEQ ID NO: 20 - DNA, 64 bp, Artificial sequence
  Synthetic construct
  MISC_FEATURE: Exemplary synthetic adaptor molecule
ctggagacgtggaggagaaccctggacctactagtgaccgctacgccccaatgacccgaccagc

SEQ ID NO: 21 - DNA, 127 bp, Artificial sequence
  Synthetic construct
  MISC_FEATURE: exemplary Type IIS restriction site at the end of the poly(A) sequence
  misc_feature (1)..(120): A residue can be absent or present
  misc_feature (120)..(120): n is a, c, g, or t
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaangaagagc

SEQ ID NO: 22 - DNA, 8066 bp, Artificial sequence
  Synthetic construct
  MISC_FEATURE: DNA template sequence for a srRNA construct within SINV AR86/Girdwood chimera 1 empty vectorwith universal adaptors
atggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaggagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaa

SEQ ID NO: 23 - DNA, 8120 bp, Artificial sequence
  Synthetic construct
  MISC_FEATURE: DNA template sequence for a srRNA construct within SINV AR86/Girdwood chimera 2 empty vectorwith universal adaptors
atggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaggagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctcgaaaagtagaaaaccagaaagccataaccactgagcgactgctttcagggctacgactgtataactctgccacagatcagccagaatgctataagatcacctacccgaaaccatcgtattccagcagtgtaccagcgaactactctgacccaaagtttgctgtagctgtttgtaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaactgccaacactggactcagcgacattcaacgttgaatgctttcgaaaatatgcatgcaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggatagattcgtcatggacatgaaaagagacgtgaaagttacacctggcacgaaacacacagaagaaagaccgaaagtacaagtgatacaagccgcagaacccctggcgaccgcttacctatgcgggatccaccgggagttagtgcgcaggcttacagccgttttgctacccaacattcacacgctctttgacatgtcggcggaggactttgatgcaatcatagcagaacacttcaagcaaggtgacccggtactggagacggatatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaa

SEQ ID NO: 24 - DNA, 8066 bp, Artificial sequence
  Synthetic construct
  MISC_FEATURE: DNA template sequence for a srRNA construct within SINV AR86/Girdwood chimera 3 empty vectorwith universal adaptors
atggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcgtaccgtactaaaagggagaacattgctgattgtcaagaggaagcagttgtcaatgcagccaatccactgggcagaccaggagaaggagtctgccgtgccatctataaacgttggccgaacagtttcaccgattcagccacagagacaggtaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaggtatcacttaactgcttgacaaccgcgctagacagaactgatgcggacgtaaccatctactgcctggataagaagtggaaggaaagaatcgacgcggtgctccaacttaaggagtctgtaactgagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgaacaactgtgtgcctacatattgggggagaccatggaagcaatccgcgaaaaatgcccggtcgaccacaacccgtcgtctagcccgccaaaaacgctgccgtgcctctgtatgtatgccatgacgccagaaagggtccacagactcagaagcaataacgtcaaagaagttacagtatgctcctccaccccccttccaaagtacaaaatcaagaatgttcagaaggttcagtgcacaaaagtagtcctgtttaacccgcatacccccgcattcgttcccgcccgtaagtacatagaagcaccagaacagcctgcagctccgcctgcacaggccgaggaggcccccggagttgtagcgacaccaacaccacctgcagctgataacacctcgcttgatgtcacggacatctcactggacatggaagacagtagcgaaggctcactcttttcgagctttagcggatcggacaactaccgaaggcaggtggtggtggctgacgtccatgccgtccaagagcctgcccctgttccaccgccaaggctaaagaagatggcccgcctggcagcggcaagaatgcaggaggagccaactccaccggcaagcaccagctctgcggacgagtcccttcacctttcttttgatggggtatctatatccttcggatcccttttcgacggagagatggcccgcttggcagcggcacaacccccggcaagtacatgccctacggatgtgcctatgtctttcggatcgttttccgacggagagattgaggagttgagccgcagagtaaccgagtcggagcccgtcctgtttgggtcatttgaaccgggcgaagtgaactcaattatatcgtcccgatcagccgtatcttttccaccacgcaagcagagacgtagacgcaggagcaggaggaccgaatactgtctaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaa

SEQ ID NO: 25 - DNA, 8120 bp, Artificial sequence
  Synthetic construct
  MISC_FEATURE: DNA template sequence for a srRNA construct within SINV AR86/Girdwood chimera 4 empty vectorwith universal adaptors
atggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacttgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagatcttgacaatgaaaaaatgctgggcaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagatgaaattggcattacaaccaaagaaggaggaaaaactgctgcaagtcccggaggaattagttatggaggccaaggctgctttcgaggatgctcaggaggaatccagagcggagaagctccgagaagcactcccaccattagtggcagacaaaggtatcgaggcagctgcggaagttgtctgcgaagtggaggggctccaggcggacaccggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaggagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaa

SEQ ID NO: 26 - DNA, 9572 bp, Artificial sequence
  Synthetic construct
  MISC_FEATURE: DNA template sequence for a srRNA construct within VEE vector containing a GOI, universal adaptors, and longer poly(A)
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactggtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctctgaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatctggagacgtggaggagaaccctggacctatggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcgagttccctagcactggcaatcatggtagctggtctatccttatggatgtgctccaatgggtcgttacaatgcagaatttgcatttgaccgctacgccccaatgacccgaccagctaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - DNA, 8179 bp, Artificial sequence
  Synthetic construct
  MISC_FEATURE: DNA template sequence for a srRNA construct within SINV Girdwood empty vector with universal adaptors
attggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggttgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattaccagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaggagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtctagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgacgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagcacatttcatctgactaataccacaacaccaccaccatgaatagaggattctttaacatgctcggccgccgccccttcccggcccccactgccatgtggaggccgcggagaaggaggcaggcggccccgggaagcggagctactaacttcagcctgctgaagcaggctggagacgtggaggagaaccctggacctactagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaa

SEQ ID NO: 28 - DNA, 18 bp, Artificial sequence
  Synthetic construct
  MISC_FEATURE: T7 RNA polymerase promoter
taatacgactcactatag

SEQ ID NO: 29 - DNA, 29 bp, Artificial sequence
  Synthetic construct
  MISC_FEATURE: T7 terminator sequence
aacccctctctaaacggaggggttttttt

SEQ ID NO: 30 - DNA, 55 bp, Artificial sequence
  Synthetic construct
  MISC_FEATURE: Forward primer
gctggagacgtggaggagaaccctggacctatggagaaaatagtgcttctttttg

SEQ ID NO: 31 - DNA, 53 bp, Artificial sequence
  Synthetic construct
  MISC_FEATURE: Reverse primer
gctggtcgggtcattggggcgtagcggtcaaatgcaaattctgcattgtaacg